(19) United States
(12) Patent Application Publication
(10) Pub. No.: US 2003/0105130 A1
(43) Pub. Date: Jun. 5, 2003

(54) Thiaporphyrin, selenaporphyrin, and carotenoid porphyrin compounds as c-myc and telomerase inhibitors

(76) Inventors: Laurence H. Hurley, Tucson, AZ (US); Tao Lu, Jiangsu (CN)

Correspondence Address: Steven L. Highlander, Fullbright & Jaworski L.L.P., Suite 2400, 600 Congress Avenue, Austin, TX, 78701, US

(21) Appl. No.: 10/109,612

(22) Filed: Mar. 28, 2002

Related U.S. Application Data
(60) Provisional application No. 60/280,556, filed on Mar. 30, 2001.

(51) Int. Cl. A61K 31/4709; A61K 31/409; A61K 31/4439; C07D 345/00

(52) U.S. Cl. 514/314; 514/410; 514/338; 540/1

(57) ABSTRACT

The present invention has identified thiaprophyrin, selenaporphyrin, and carotenoid porphyrin compounds that bind the G-quadruplex formed by the folding of single-stranded human telomeric DNA. These compounds have been shown to be effective telomerase and c-myc inhibitors and are contemplated to be useful in developing cancer treatments.

This application claims priority to and specifically incorporates by reference, the content of U.S. Provisional Application Serial No. 60/280,556 filed Mar. 30, 2001.

The government has rights in the following invention pursuant to National Cancer Institute Grant CA67760.

### BACKGROUND OF THE INVENTION

I. Field of the Invention

This invention relates generally to the field of cancer therapy. More particularly, the present invention relates to thiaporphyrin, selenaporphyrin, and carotenoid porphyrin compositions and their use as c-myc and telomerase inhibitors.

II. Description of the Related Art

Telomeres consist of characteristic tandem repeats (TTAGGG in humans) found at the ends of most eukaryotic chromosomes (Blackburn, 1991). The stability and integrity of eukaryotic chromosomes depend on these genetic elements, which are synthesized by the ribonucleoprotein enzyme telomerase, a specialized reverse transcriptase that contains its own RNA template for synthesis of telomeric DNA (Greider and Blackburn, 1989; Shippen-Lentz and Blackburn, 1990). The activity of this telomerase has been associated with cancer cells (Kim et al., 1994) and is thus a potential target for anticancer chemotherapy. In fact, significant levels of telomerase activity have been detected in over 85% of tumors (Kim et al., 1994). Telomerase is also present in stem and germline cells of normal tissues, albeit at much lower levels (Morin, 1995). Thus, telomerase presents a target with potentially good selectivity for tumor over healthy tissue (Morin, 1995; Parkinson, 1996; Raymond et al., 1996).

The structure of the human telomerase protein remains elusive, although recently shown to be closely related to other reverse transcriptases (Linger et al., 1997). However, it has been possible to inhibit telomerase activity either by antisense strategies directed towards the telomerase RNA template, for example, peptide nucleic acids (Norton et al., 1996) and phosphorothioate oligonucleotides (Mata et al., 1997) or by using inhibitors of reverse transcriptases e.g., established agents such as AZT (Strahl and Blackburn, 1996) and other nucleosides (Fletcher et al., 1996)). Inhibition by cisplatin, possibly due to crosslinking of the telomeric repeat sequences, has also been reported (Burger et al., 1997).

C-myc and mad also play critical roles in proliferation and differentiation, respectively. These effects are mediated by dimerization with max to form either c-myc-max or mad-max leucine zippers that target transcriptional regulatory regions in a variety of downstream genes. C-myc controls levels of hTERT, the catalytic subunit of telomerase. The relative amounts of c-myc and mad determine the proliferative or differentiation capability of cells. In many types of cancers, c-myc expression is deregulated due to chromosomal translocation or gene amplification, and in many cases the major promoter involved in control of gene expression is the P1 promoter. The deregulated expression of c-myc occurs in many human cancers such as lymphomas, leukaemias, and lung, cervical, ovarian, breast, and gastric cancers.

A mechanism for telomere synthesis by telomerase has been proposed by Blackburn and co-workers (Greider and Blackburn, 1989; Shippen-Lentz and Blackburn, 1990). In this mechanism, the processivity of telomere synthesis depends on translocation of the growing telomere. Although the exact mechanism of translocation is not yet well understood, this step appears to involve unwinding of the DNA:RNA hybrid formed by the extended telomere at the start site on the template. Since translocation can occur in the absence of a high-energy cofactor, it has been proposed that the formation of either G:G hairpin or G-quadruplex structures by the telomere product may provide the driving force for translocation (Shippen-Lentz and Blackburn, 1990; Zhaler et al., 1991).

The unique nucleic acid structures associated with telomeric DNA have been proposed as targets for the design of telomerase inhibitors (Zhaler et al, 1991; Shippen-Lentz and Blackburn, 1990). Other studies on the unique DNA secondary structures adopted by telomeric DNA sequences have been reported (Fletcher et al, 1996; Salazar et al, 1996). Several reviews on G-quadruplexes as a target for drug design have appeared (Han and Hurley, 2000; Kerwin, 2000; Neidle et al., 2000).

The telomeres are multiple tandem repeats of a highly conserved DNA sequence (in mammals 5′-TTAGGG-3′) (SEQ ID NO: 3) found at the ends of chromosomes and in human germline cells the telomeres may be 15-25 kilobases long. The telomeres are dynamic structures responsible for chromosome stability and have a role in control of chromosome separation and are thus involved in regulation of the cell cycle. The end replication problem means that with each cell division about 60-100 bases are lost from the ends of the chromosomes and as the telomeres shorten, cells eventually reach crisis and apoptosis is triggered. In immortal cell lines (tumors, germline and stem cells), an unusual enzyme activity—telomere terminal transferase, telomerase—is active which maintains the telomere length just above the crisis level. Whether telomerase activation is a cause or effect of the neoplastic state remains a matter of debate. However, the observation that telomerase is active in almost all tumor cells but not in most normal tissues does mean that telomerase presents a potentially highly selective target for the design of new agents to interfere with the growth of tumor cells.

It has previously been shown that cationic porphyrins based on 5,10,15,20-tetra(N-methyl-4-pyridyl)porphyrin chloride (TMPyP4) can stabilize G-quadruplex DNA and consequently inhibit human telomerase in a cell-free system (U.S. Pat. No. 6,087,493). The solution structure of a twenty-two-base oligonucleotide based on the human telomerase sequence, d(AG3[T2AG3]3), has been solved (Wang et al., 1993; Wheelhouse et al., 1998; Arthanari et al., 1998; Anantha et al., 1998). It consists of a single looped strand, which is stabilized by a core of stacked G-tetrads. Using coordinates from the solution structure of the quadruplex and crystal structure of the porphyrin combined with experimentally derived stoichiometry (Wang et al., 1993), a minimized model of the 2:1 TMPyP4: d(AG3[T2AG3]3) complex was built. The quadruplex could accommodate porphyrins above and below the tetrads at the core of the complex with little distortion. Moreover, monovalent cations such as K+ and Na+ have been shown to stabilize G-quadruplex structures, presumably by coordinating with the eight carbonyl oxygen atoms present between stacked tetrads (Wheelhouse et al. 1998; Arthanari et al. 1998; Anantha et al., 1998).

It is theorized that these cationic porphyrins do not inhibit telomerase directly, but instead do so though a mechanism involving the inhibition of c-myc. The myc family of oncogenes encodes proteins that are responsible for activating telomerase.

The formation of DNA tetraplexes or G-quadruplexes are necessary to activate the c-myc gene. It is thought that cationic porphyrins bind to the human G-quadruplex structure and inhibit the expression of c-myc. This in turn results in the down regulation of telomerase.

A wide range of TMPyP4 analogues have previously been synthesized and assayed against telomerase (U.S. Pat. No. 6,087,493). However, they have the potential problem of photo-induced skin toxicity, which may affect their clinic use. A need therefore exists for compounds that can inhibit telomerase, but do not pose the problem of photo-induced skin toxicity.

### SUMMARY OF THE INVENTION

The present invention describes a novel class of core modified porphyrins that are c-myc and telomerase inhibitors. The compounds of the present invention also exhibit no photoinduced skin toxicity. More specifically, various thiaporphyrins and selenaporphyrins with cationic electron withdrawing groups are disclosed. In addition, carotenoid porphyrins are also disclosed that inhibit c-myc and telomerase and also exhibit no photoinduced skin toxicity.

In one embodiment, a method of inhibiting the expression of c-myc in a cell is disclosed. The method comprises contacting a cell with a thiaporphyrin or a selenaporphyrin with the following formula:

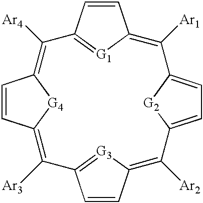

where one of G1, G2, G3, or G4 is S or Se, and the remainder are N,N, and NH, or where two of G1, G2, G3, or G4 are either both S or both Se, two are N, and the two N are located opposite each other.

A metal may also be coordinated to the thiaporphyrin or the selenaporphyrin, such that the thiaporphyrin or the selenaporphyrin has a formula:

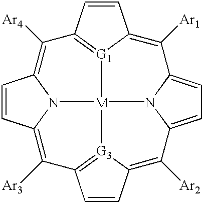

where G1 is S or Se and G3 is N, or where G1 and G3 are both S or both Se. M is a metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

Ar1, Ar2, Ar3 and Ar4 may be H or may independently be:

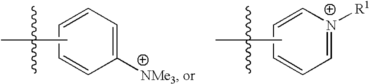

where R1 is H, lower alkyl, —CH2CH2OH, CH₂OAc, or —CH2CH2CH2SO3—,

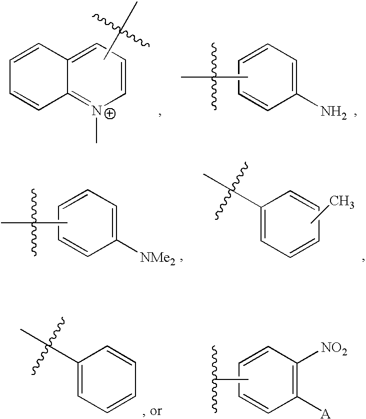

where A is H, OH, OMe, Cl or Me,

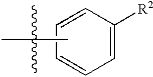

where R2 is —CO2H, CONH2, CONHCH2CH2Br or NHCOCH3.

In another embodiment, Ar1, Ar2, Ar3 and Ar4 may independently be:

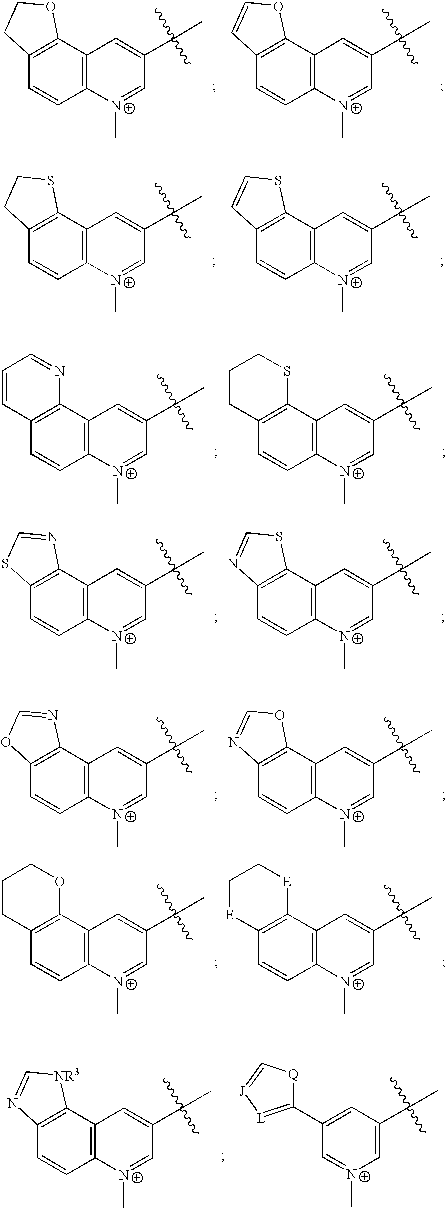

where Q is O, S, NH or NMe; J is CN or N; L is N or CH; R3 is lower alkyl; and each E is independently CH2, NH, NMe, O or S.

Alternatively, Ar1, Ar2, Ar3 and Ar4 may independently be:

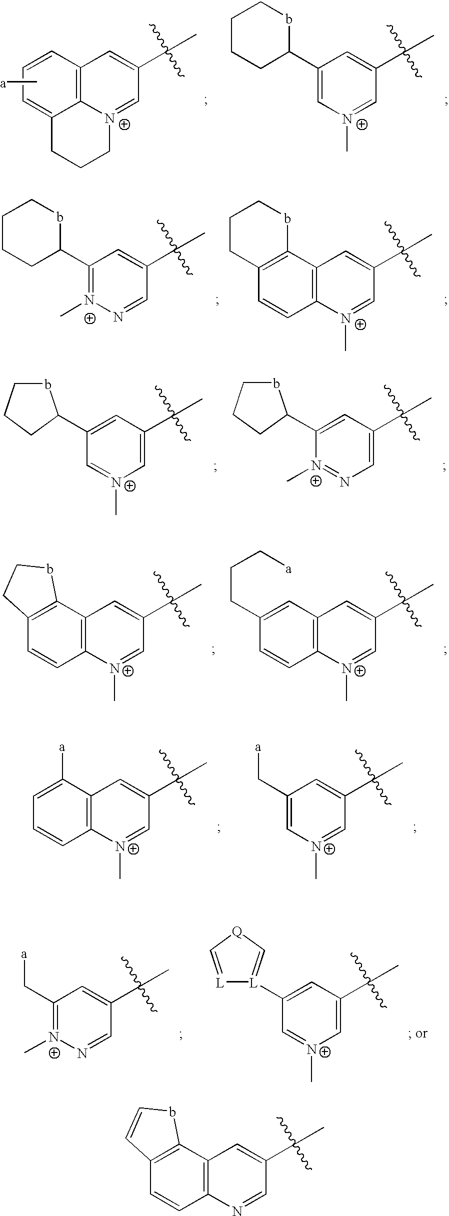

where a is NH₂, NHME, NMe₂, OH, OMe, Sme; b is NH, NMe, SMe, O or S; Q is O, S, NH or NMe; each L is independently N or CH

Ar1, Ar2, Ar3 and Ar4 may also independently be:

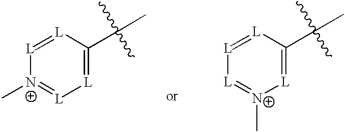

where each L is independently N or CH.

Ar1, Ar2, Ar3 and Ar4 may also independently be:

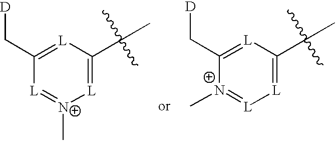

where each L is independently N or CH and D is NH2, NHMe, NMe2, OH, SH, SMe or CF3.

In yet another embodiment of the invention, Ar1, Ar2, Ar3 and Ar4 may independently be:

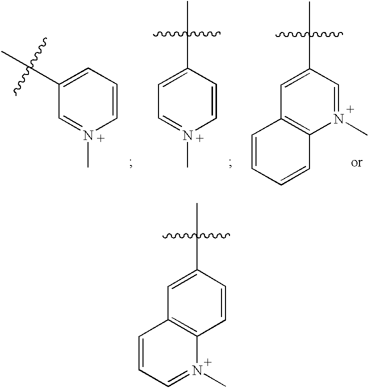

At least one, but not more than two, of Ar1, Ar2, Ar3, or Ar4 may also be

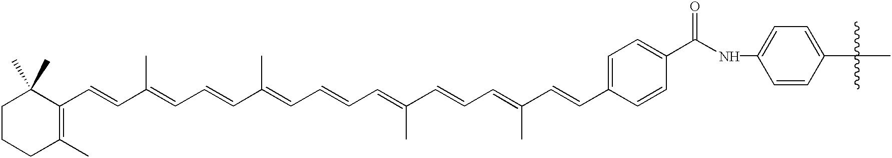

and the remainder of Ar1, Ar2, Ar3, or Ar4 are positively charged moieties.

Ar1, Ar2, Ar3 and Ar4 may also independently be

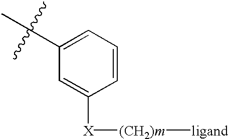

where m is 0-3, X is O, NH, CO, or CH2, and where ligand is:

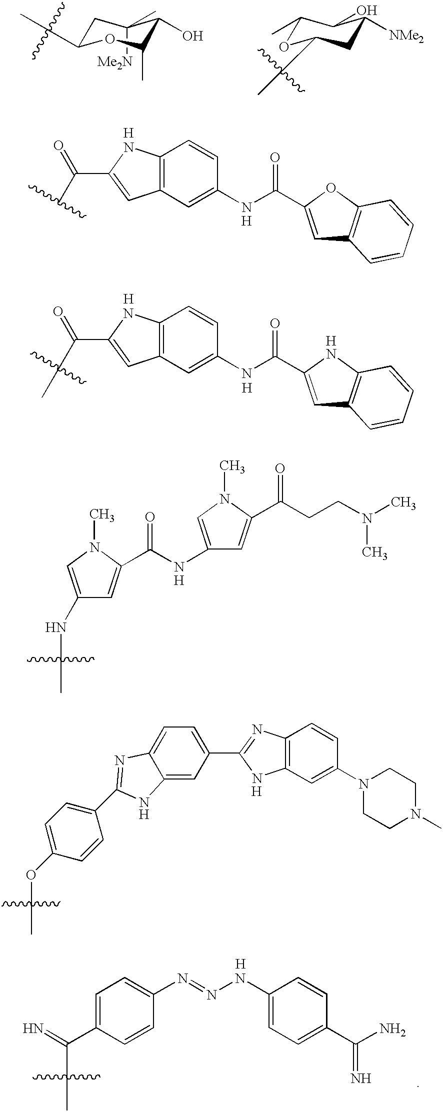

In a further embodiment of the invention, at least one, but not more than two, of Ar1, Ar2, Ar3, or Ar4 may also be

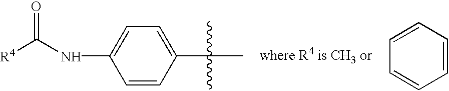

and the remainder of Ar1, Ar2, Ar3, or Ar4 are positively charged moieties. “Positively charged moieties,” as used throughout the present application, may include any of the positively charged moieties disclosed herein. Additionally, other positively charged moieties that may be used in the present invention will be apparent to those skilled in the art.

A method for cleaving telomeric DNA is also disclosed. The method comprises contacting the telomeric DNA with a thiaporphyrin or a selenaporphyrin having a formula:

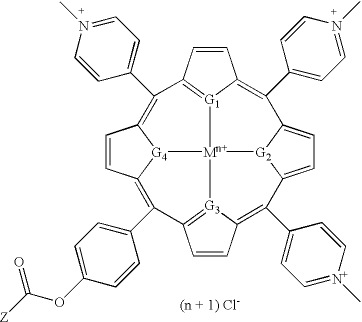

where one of G1, G2, G3, or G4 is S or Se, and the remainder are N, or where two of G1, G2, G3, or G4 are either both S or both Se, two are N, and the two N are located opposite each other. Z may be Fe.EDTA, n is 1-3, and M is H+ or metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

In another embodiment, a method for covalently modifying telomeric DNA is disclosed. The method comprises contacting telomeric DNA with a thiaporphyrin or a selenaporphyrin having a formula:

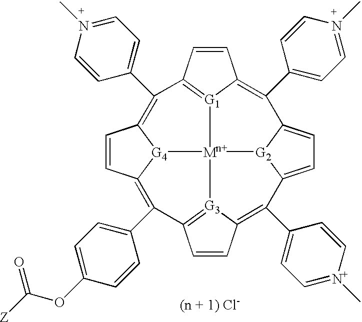

where one of G1, G2, G3, or G4 is S or Se, and the remainder are N, or where two of G1, G2, G3, or G4 are either both S or both Se, two are N, and the two N are located opposite each other. Z may be

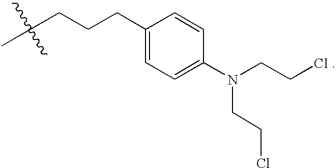

M is H+ or a metal cation selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu, and n is the charge on the metal M. In a further embodiment of this method, the telomeric DNA is a G-quadruplex. The telomeric DNA may be human telomeric DNA.

In yet another embodiment, a compound is disclosed having the following formula:

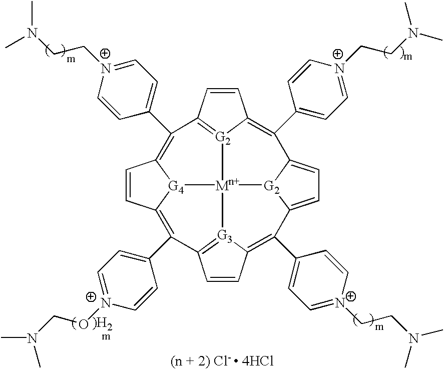

where one of G1, G2, G3, or G4 is S or Se, and the remainder are N, or where two of G1, G2, G3, or G4 are either both S or both Se, two are N, and the two N are located opposite each other. M is H+ or a metal cation selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu, m is 0-3, and n is the charge on the metal ion M.

In a further embodiment, a compound is disclosed having the following formula:

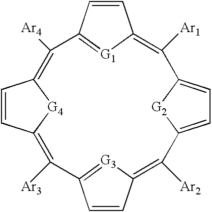

where one of G1, G2, G3, or G4 is S or Se, and the remainder are N,N, and NH, or where two of G1, G2, G3, or G4 are either both S or both Se, two are N, and the two N are located opposite each other. The compound may also comprise a metal, M, coordinated to the thiaporphyrin or the selenaporphyrin. M may be selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu and salts thereof. Pharmaceutical compositions are also contemplated that comprise these compounds.

In one embodiment, Ar1, Ar2, Ar3 and Ar4 are H or independently

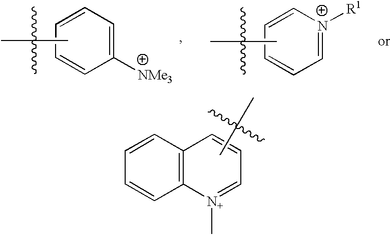

where R1 is H, CH3, lower alkyl, —CH2CH₂OH, CH₂OAc, or —CH2CH2CH2SO3—,

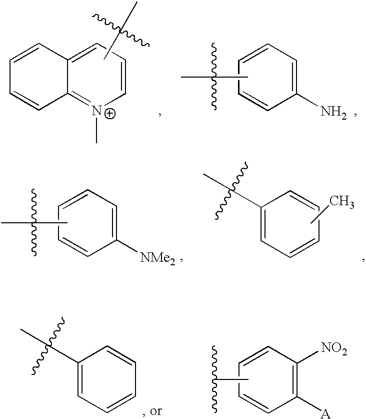

where A is H, OH, OMe, Cl or Me,

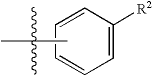

R2 is —CO2H, CONH2, CONHCH2CH2Br or NHCOCH3 and salts thereof.

Ar1, Ar2, Ar3 and Ar4 may also independently be:

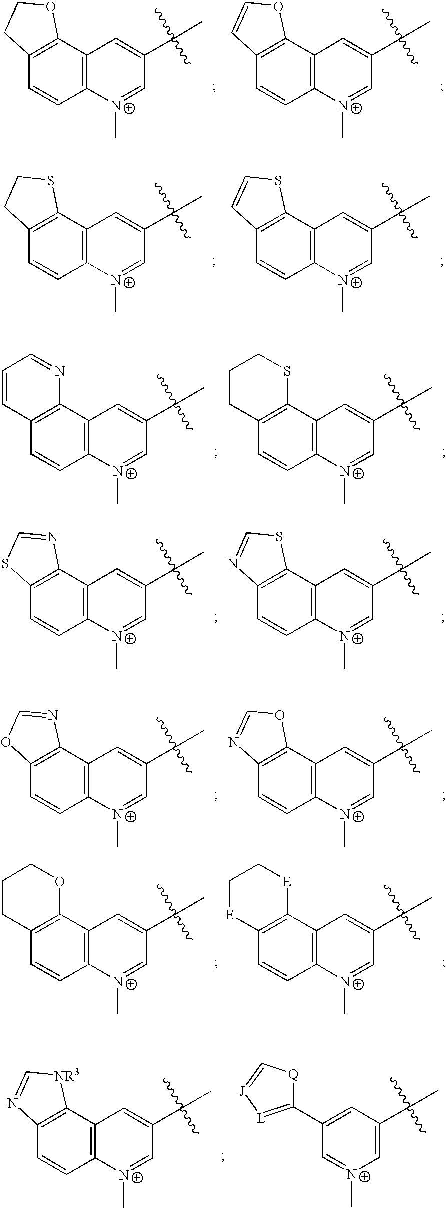

where Q is O, S, NH or NMe; J is CN or N; and L is N or CH. R3 is lower alkyl, and each E is independently CH2, NH, NMe, O or S.

In a another embodiment, Ar1, Ar2, Ar3 and Ar4 are independently:

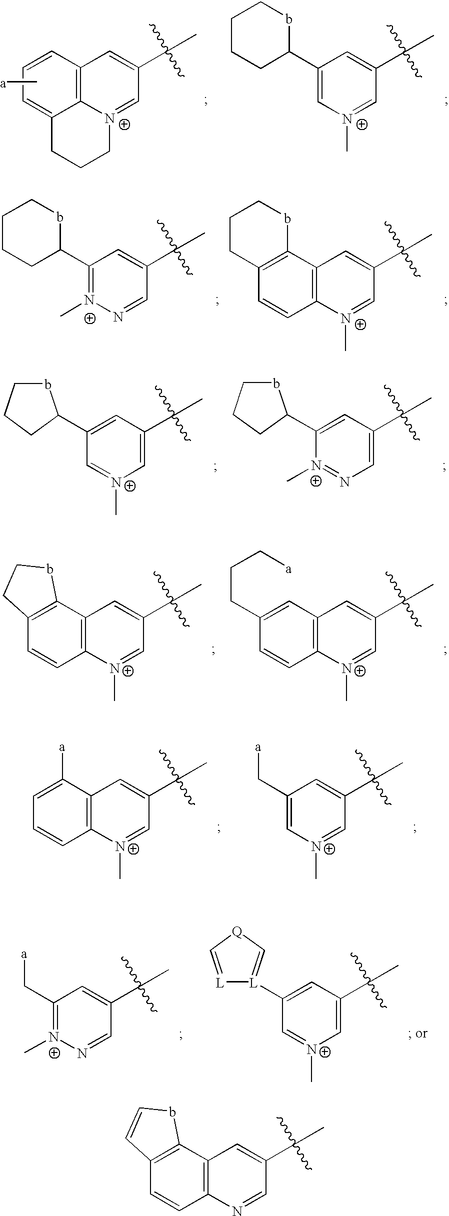

where a is NH₂, NHMe, NMe₂, OH, OMe, Sme; b is NH, NMe, SMe, O or S; Q is O, S, NH or NMe; each L is independently N or CH

Ar1, Ar2, Ar3 and Ar4 may also independently be:

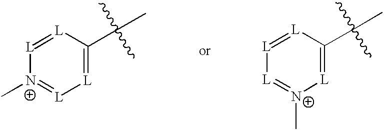

where each L is independently N or CH. Alternatively, Ar1, Ar2, Ar3 and Ar4 may independently be:

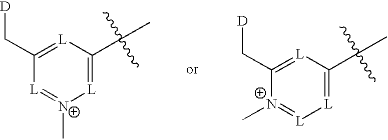

where each L is independently N or CH and D is NH2, NHMe, NMe2, OH, SH, SMe or CF3.

Ar1, Ar2, Ar3 and Ar4 may also independently be:

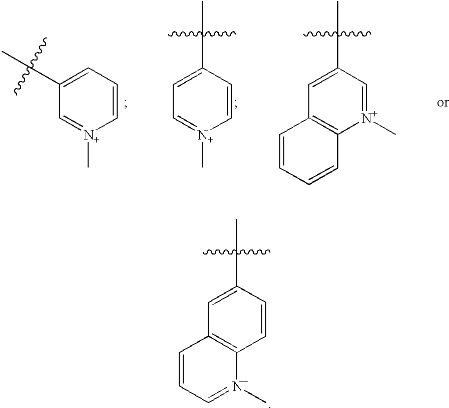

In another embodiment, at least one, but not more than two, of Ar1, Ar2, Ar3, or Ar4 is

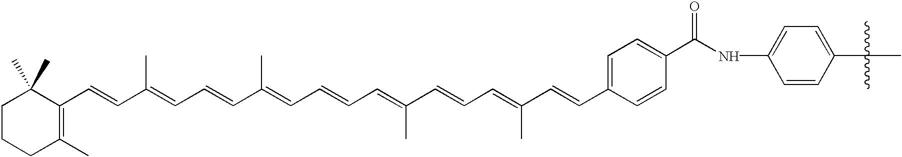

and the remainder of Ar1, Ar2, Ar3, or Ar4 are positively charged moieties. Alternatively, at least one, but not more than two, of Ar1, Ar2, Ar3, or Ar4 may be:

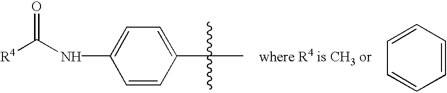

and the remainder of Ar1, Ar2, Ar3, or Ar4 are positively charged moieties.

In particular, the following compounds are disclosed: 5,10-bis(N-methyl-6-quinolyl)-15,20-bis(N-methyl-4-pyridyl)-21-monothiaporphyrin chloride; 5,10-bis(N-methyl-3-pyridyl)-15,20-bis(N-methyl-4-pyridyl)-21-monothiaporphyrin chloride; 5,10,15,20-tetra(N-methyl-3-pyridyl)-21-monothiaporphyrin chloride, 5,10,15,20-tetra(N-methyl-6-quinolyl)-21,23-dithiaporphyrin chloride; 5,10,15,20-tetra(N-methyl-3-pyridyl)-21,23-dithiaporphyrin chloride; and 5,10,15,20-tetra(N-methyl-3-quinolyl)-21,23-dithiaporphyrin chloride.

A method is also disclosed for inhibiting cell proliferation by contacting a cell with an effective amount of a thiaporphyrin or a selenaporphyrin. The cell may be in a mammal, and may be a cancer cell. The cancel cell may be, for example, a prostate or lymphoma cell. The cancer cell may also be a breast cancer cell, and particularly may be BT20, MCF-7m, 11 S578t, HS576Bst or Hela cell.

In another embodiment, Ar1, Ar2, Ar3, or Ar4 may be as follows:

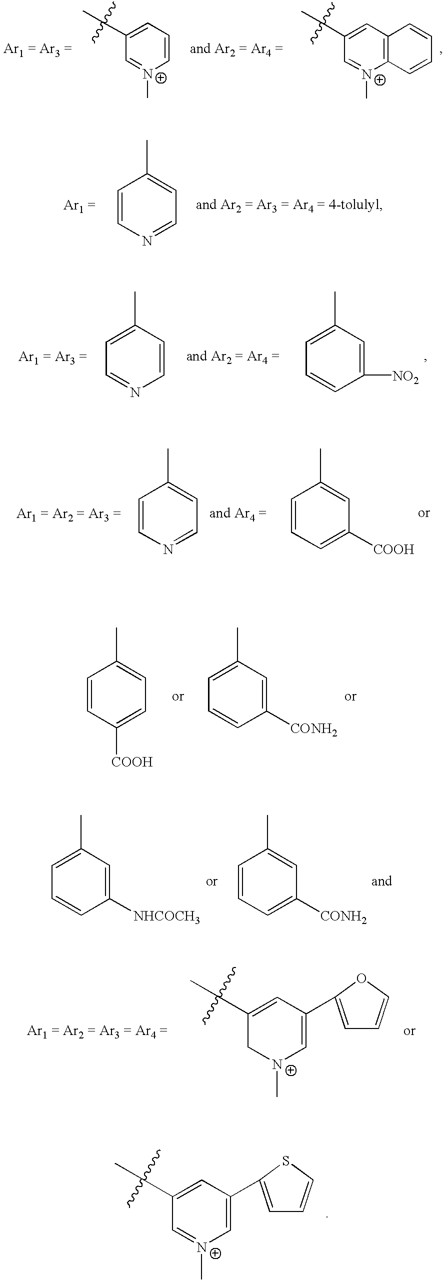

In a yet a further embodiment, Ar1, Ar2, Ar3, or Ar4 may be as follows:

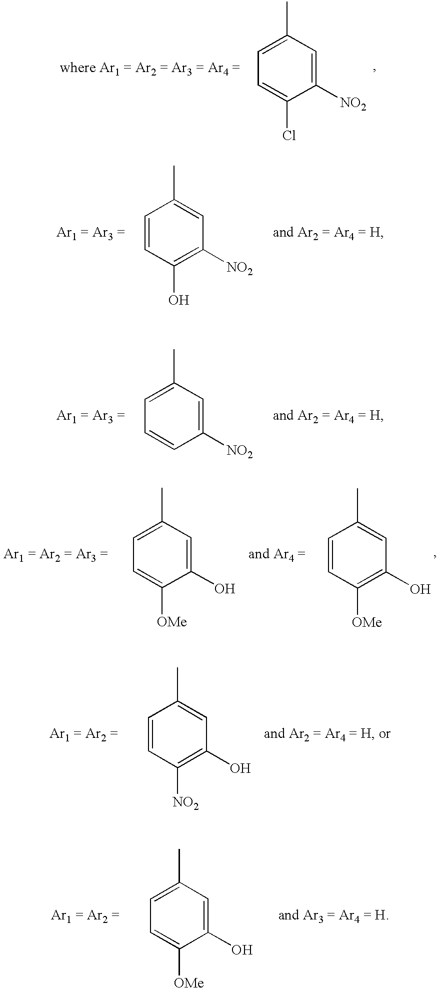

A thiaporphyrin or a selenaporphyrin with the following formula is also disclosed:

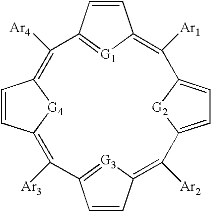

where one of G1, G2, G3, or G4 is S or Se, and the remainder are N,N, and NH, or where two of G1, G2, G3, or G4 are either both S or both Se, two are N, and the two N are located opposite each other. Ar1, Ar2, Ar3, and Ar4 are positively charged moieties, and assume a nonplanar disposition with regard to said thiaporphyrin structure or said selenaporphyrin structure. A metal may also be coordinated to the thiaporphyrin or the selenaporphyrin. A method of inhibiting the expression of c-myc in a cell, comprising contacting the cell with the thiaporphyrin or selenaporphyrin is also disclosed. Additionally, a method is disclosed for inhibiting proliferation of a cell comprising contacting said cell with the thiaporphyrin or selenaporphyrin.

The present invention also teaches a compound having the formula:

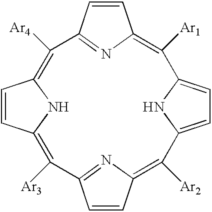

wherein at least one, but not more than two, of Ar1, Ar2, Ar3, or Ar4 is

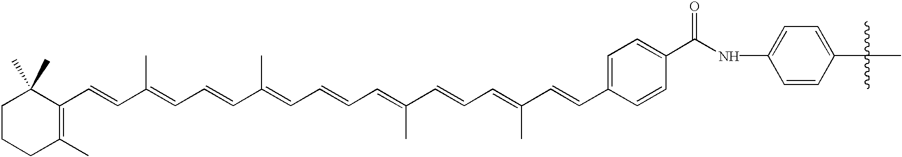

or where at least one, but not more than two, of Ar1, Ar2, Ar3, or Ar4 is

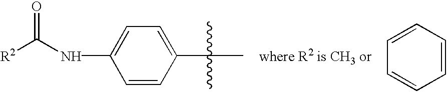

and the remainder are positively charged moieties. The compound may also be coordinated to a metal, M, where M is 2H+ or a metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

The following compounds, for example, are disclosed: 5-{4-[4-(7′-apo-7′-β-carotenyl)benzoylamido]phenyl}-10,15,20-tri(N-methyl-4-pyridyl)porphyrin chloride; 5-{4-[4-(7′-apo-7′-β-carotenyl)benzoylamido]phenyl}-10,15,20-tri(N-methyl-3-pyridyl)porphyrin chloride; 5-{4-[4-(7′-apo-7′-β-carotenyl)benzoylamino]phenyl}-10,15,20-tri(N-methyl-3-quinolyl)porphyrin chloride; 5-(4-acetamidophenyl)-10,15,20-tri(N-methyl-4-pyridyl)porphyrin chloride; 5-(4-acetamidophenyl)-10,15,20-tri(N-methyl-3-pyridyl)porphyrin chloride; 5-(4-acetamidophenyl)-10,15,20-tri(N-methyl-3-quinolyl)porphyrin chloride; 5-(4-benzoylamidophenyl)-10,15,20-tri(N-methyl-4-pyridyl) porphyrin chloride; 5-(4-benzoylamidophenyl)-10,15,20-tri(N-methyl-3-pyridyl)-porphyrin chloride; and 5-(4-benzoylamidophenyl)-10,15,20-tri(N-methyl-3-quinolyl)-porphyrin chloride.

In yet another embodiment, a method of modifying telomerase or telomere function is disclosed. The method comprises contacting a thiaporphyrin or a selenaporphyrin with telomeric DNA wherein the thiaporphyrin or the selenaporphyrin has a formula:

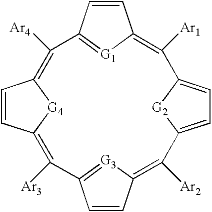

where one of G1, G2, G3, or G4 is S or Se, and the remainder are N,N, and NH or where two of G1, G2, G3, or G4 are either both S or both Se, two are N, and the two N are located opposite each other. Ar1, Ar2, Ar3 and Ar4 may be H or may independently be:

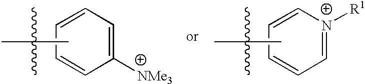

where R1 is H, lower alkyl, —CH2CH2OH, CH2OAc, or —CH2CH2CH2SO3—,

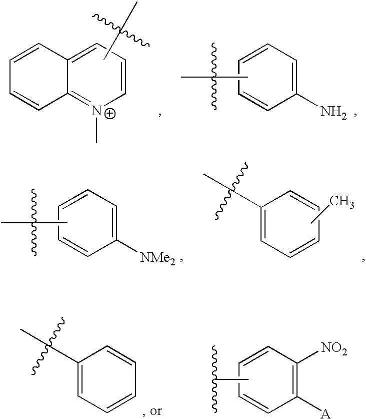

A is H, OH, OMe, Cl or Me,

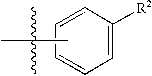

and R2 is —CO2H, CONH2, CONHCH2CH2Br or NHCOCH3.

Ar1, Ar2, Ar3 and Ar4 may also independently be:

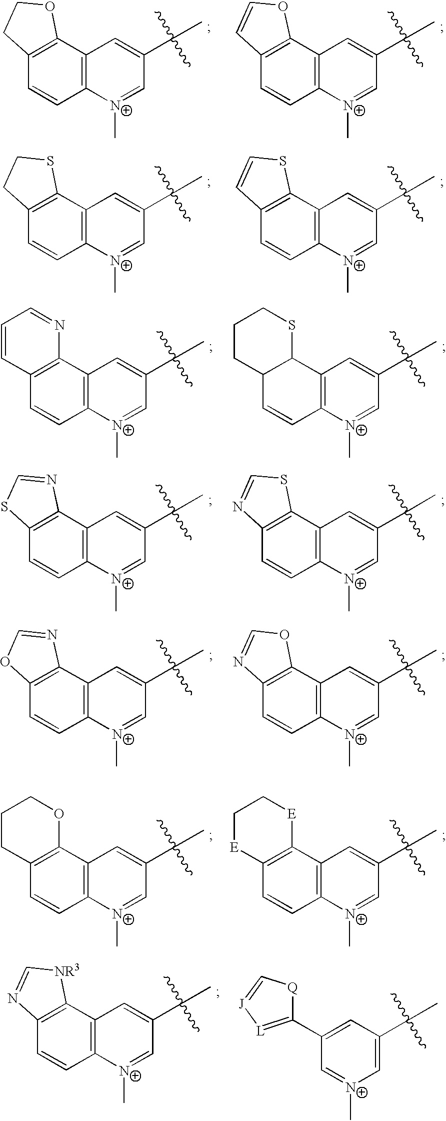

where Q is O, S, NH or NMe; J is CN or N; L is N or CH; where R3 is lower alkyl, and each E is independently CH2, NH, NMe, O or S.

In yet another embodiment, Ar1, Ar2, Ar3 and Ar4 are independently:

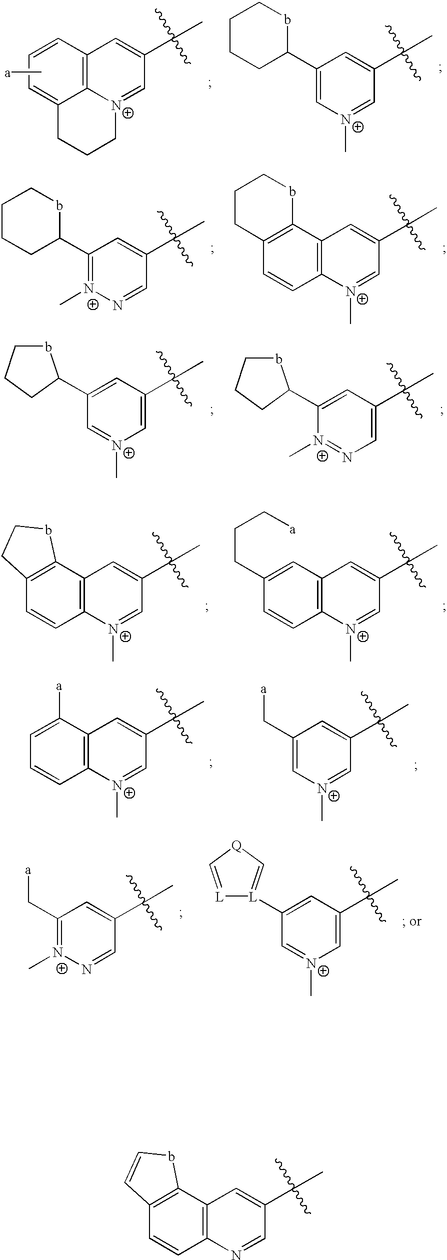

where a is NH2, NHMe, NMe₂, OH, OMe, Sme; b is NH, NMe, SMe, O or S; Q is O, S, NH or NMe; each L is independently N or CH

Ar1, Ar2, Ar3 and Ar4 may also be independently:

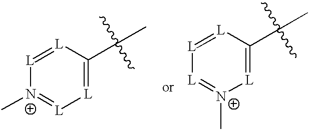

where each L is independently N or CH.

Alternatively, Ar1, Ar2, Ar3 and Ar4 are independently:

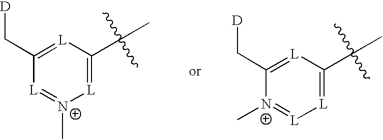

where each L is independently N or CH and D is NH2, NHMe, NMe2, OH, SH, SMe or CF3.

In a further embodiment, Ar1, Ar2, Ar3 and Ar4 are independently:

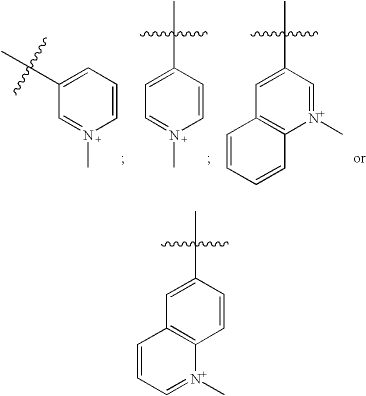

In a different embodiment, at least one, but not more than two, of Ar1, Ar2, Ar3, or Ar4 may be:

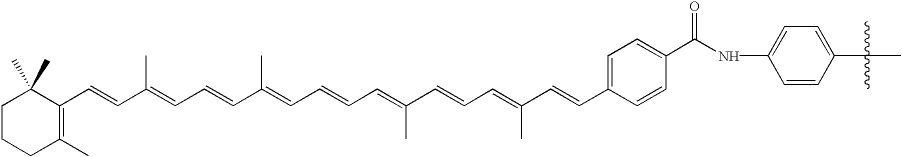

and the remainder of Ar1, Ar2, Ar3, or Ar4 are positively charged moieties. Finally, at least one, but not more than two, of Ar1, Ar2, Ar3, or Ar4 may be

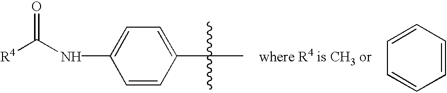

and the remainder of Ar1, Ar2, Ar3, or Ar4 are positively charged moieties.

A method of modifying telomerase or telomere function is also disclosed. The method comprises contacting a thiaporphyrin or a selenaporphyrin with telomeric DNA wherein the thiaporphyrin or the selenaporphyrin has a formula:

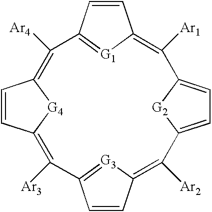

where one of G1, G2, G3, or G4 is S or Se, and the remainder are N,N, and NH or where two of G1, G2, G3, or G4 are either both S or both Se, two are N, and the two N are located opposite each other. Ar1, Ar2, Ar3 and Ar4 may be independently:

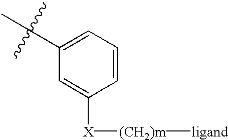

where m is 0-3, X is O, NH, CO, or CH2, and where ligand is:

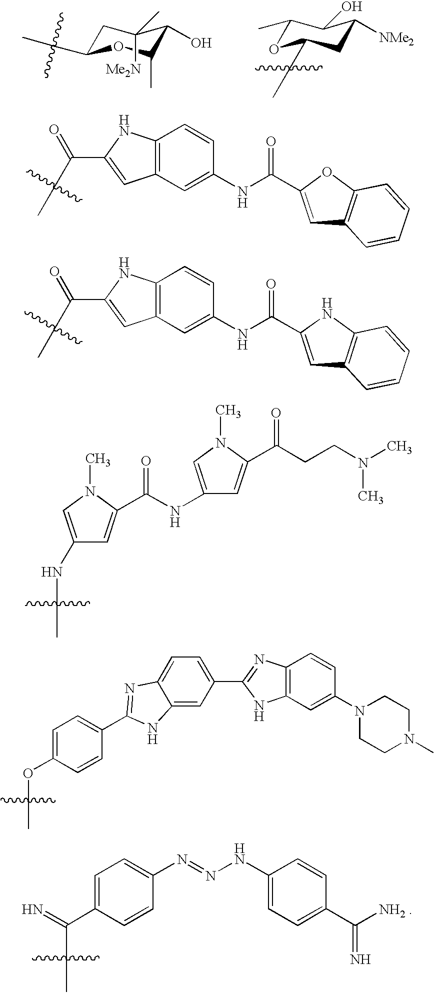

Ar1, Ar2, Ar3, and Ar4 may also be any positively charged moieties that assume a nonplanar disposition with regard to said thiaporphyrin structure or said selenaporphyrin structure.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a model for the involvement of G-quadruplex structures in c-myc transcriptional activation.

FIG. 2 illustrates the effects of TMPyP2 and TMPyP4 treatment on gene expression in MiaPaCa-2 pancreatic cancer cells.

FIG. 3 illustrates the effects of TMPyP2 and TMPyP4 treatment on c-myc protein levels in MiaPa Ca-2 pancreatic cell lines.

FIG. 4 illustrates the effect of TMPyP2 and TMPyP4 on telomere shortening.

FIG. 5 illustrates the effect of TMPyP2 and TMPyP4 on cell growth inhibition I multiple myeloma cells.

FIG. 6A illustrates four stranded I-motifs in which cytidine/cytidine pairs are interdigitated. FIG. 6B illustrates the structure of TMPyP4.

FIG. 7 illustrates a model of the TMPyP4-1-motif complex.

FIG. 8 illustrates a cartoon of the G-quadruplex with K+ (K+), the G1 complex (G1), the G2 complex (G2), and TMPyP4.

### DETAILED DESCRIPTION OF ILLUSTRATIVE EMBODIMENTS

1. The Present Invention

The activity of c-myc and telomerase has been associated with cancer cells and are thus potential targets for anticancer chemotherapy. C-myc controls levels of hTERT, the catalytic subunit of telomerase. In many types of cancers, c-myc expression is deregulated due to chromosomal translocation or gene amplification. This deregulation of c-myc results in an increase in the level of telomerase activity. Significant levels of telomerase activity have been detected in over 85% of tumors.

A wide range of TMPyP4 analogues have previously been synthesized and assayed against telomerase (U.S. Pat. No. 6,087,493). It is thought that these cationic porphyrins bind to the human G-quadruplex structure, one of the structures necessary to activate the c-myc gene. This results in the inhibition of the expression of c-myc, and in turn results in the down regulation of telomerase. However, these compounds have the potential problem of photo-induced skin toxicity, which may affect their clinic use.

The present invention discloses various thiaporphyrin, selenaporphyrin, and carotenoid porphyrin compounds. These compounds are effective in inhibiting c-myc and telomerase, and are therefore promsing anticancer agents. These compounds are particlulary advantageous because they do not exhibit photoinduced skin damage.

II. Thiaporphyrins, Selenaporphyrins, and Carotenoid Porphyrins as G-Quadruplex Interactive Compounds

Various compounds are disclosed that will selectively inhibit c-myc and telomerase by targeting nucleic acid structures, such as G-quadruplex. As previously explained, the primary c-myc control element forms structures known as G-quadruplexes, and the formation of these structures are necessary to activate the c-myc gene. The c-myc gene in turn encodes proteins that are responsible for activating telomerase. The ability of cationic porphyrins to downregulate c-myc most probably is as a result of interaction with DNA secondary structures such as G-quadruplexes. The inhibition of the expression of c-myc results in the regulation of telomerase.

The identification of such G-quadruplex interactive agents is an efficient approach for identifying c-myc and human telomerase inhibitors. Compounds, such as various porphyrins, that are believed to interact with G-quadruplex structures are known in the art. However, many of these compounds have the potential problem of photo-induced skin toxicity. The thiaporphyrins, selenaporphyrins, and carotenoid porphyrins of the present invention are advantageous because they have no photo-induced skin toxicity.

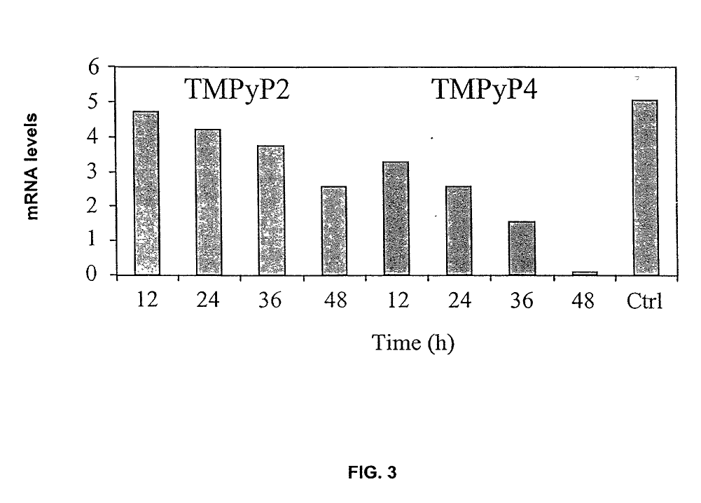

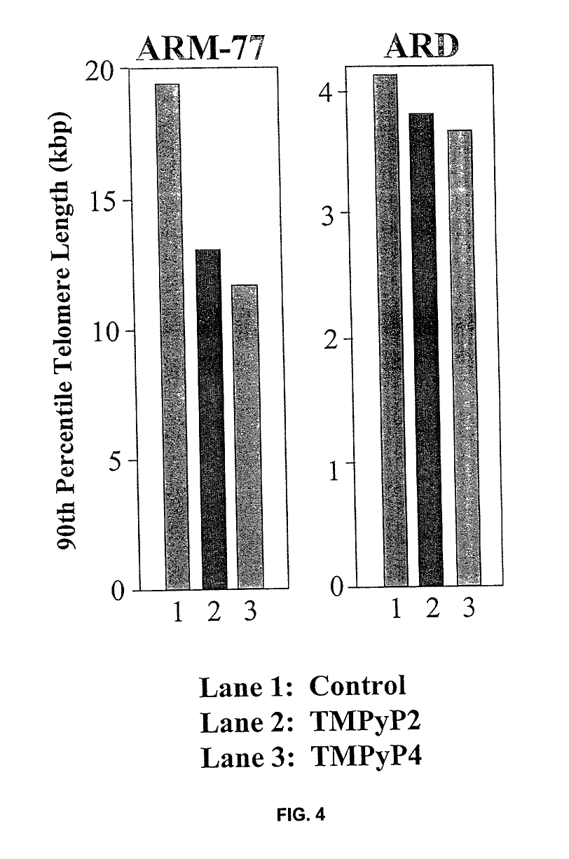

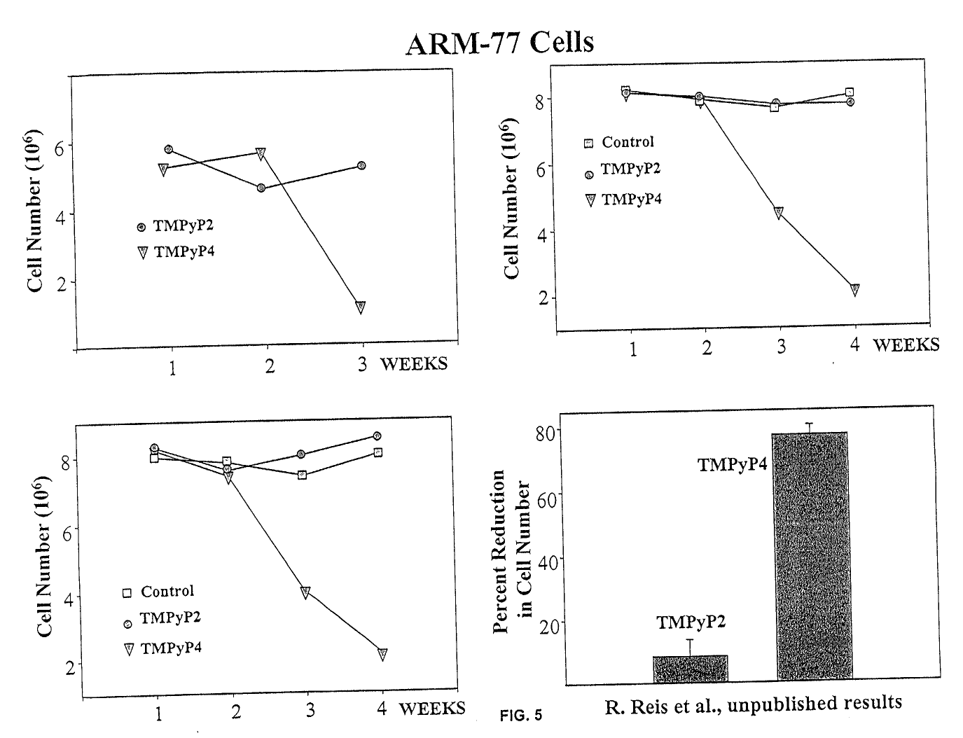

Results illustrating the ability of cationic porphyrins such as TMPyP4 to downregulate c-myc are shown in FIG. 3. The same compounds can interact with G-quadruplex formed in the telomeric regions and both of these effects lead to a compromised ability of the cell to maintain telomere length. Thus treatment of multiple myeloma cells with TMPyP4 leads to telomere shortening cell crisis and apoptosis. TMPyP2 has a much lesser effect, as illustrated in FIGS. 4 and 5.

It is envisioned that the telomerase inhibitors will provide therapy for tumors and cancers including skin cancers, connective tissue cancers, adipose cancers, breast cancers, lung cancers, stomach cancers, pancreatic cancers, ovarian cancers, cervical cancers, uterine cancers, anogenital cancers, kidney cancers, bladder cancers, colon cancers, prostate cancers, central nervous system (CNS) cancers, retinal cancer, blood, lymphoid cancers and the like.

III. C-myc

In many types of cancers, c-myc expression is deregulated due to chromosomal translocation or gene amplification, and in many cases the major promoter involved in control of gene expression is the P1 promoter. There is a purine/pyrimidine-rich region located 115 bases upstream of the P1 promoter and this element is responsible for 75-85% of total c-myc transcription (Berberich et al., 1995; Davis et al., 1989). This DNA segment is highly sensitive to nuclease and chemical modification (Siebenlist et al., 1984) and has been termed NHE (nuclease hypersensitive element). The NHE has a high potential to form a typical DNA structures under superhelical stress. Formation and stabilization of these structures may be important for gene regulation. Indeed, a colinear triplex formed between a site-specific oligonucleotide and duplex DNA can repress c-myc transcription in vitro (Postel et al., 1991). Alteration of NHE structure can influence binding of both negative and positive transcription factors. For example, the positive transcription factor hnRNP K binds sequence specifically to the pyrimidine-rich strand of NHE and has double-helix destabilizing properties (Michelotti et al., 1996).

The marked disparity in the nucleotide composition of two of the strands of NHE prompted H-DNA as a model of the noncanonical NHE structure. H-DNA involves an intramolecular pyrimidine-purine-pyrimidine triplex. Alternatively, the purine-pyrimidine-purine triplex has proposed as an explanation for the observed nuclease hypersensitivity. Both of these structures require nonphysiological conditions to be stable, either low pH for the pyrimidine triplex or very high magnesium concentration for the purine triplex, and are highly unlikely to form in vivo (Simonson et al., 1998).

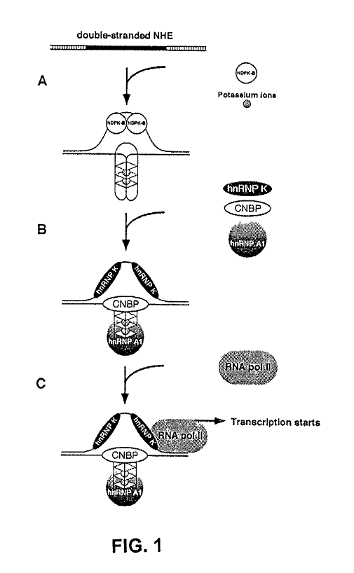

Recently, the NHE fragment of DNA has been shown to adopt an intrastrand fold-back DNA tetraplex under physiological conditions (Simonson et al., 1998). According to the proposed model, an interconversion of the NHE between a normal B-DNA conformation and a very stable a typical G-quadruplex DNA conformation can recruit transcription factors and activate the c-myc transcription. See FIG. 1. Involvement of G-quadruplex structures in the regulation of c-myc transcription opens an interesting area for the design of small molecules that can selectively interact with the G-quadruplex structure. Many G-quadruplex-interactive agents have been developed and are shown to promote and/or stabilize these secondary DNA structures (Han and Hurley et al., 2000). TMPyP4 is a small molecule from the class of porphyrin compounds that has been well characterized for G-quadruplex interaction. It has been shown to act as a driver in accelerating the assembly of G-quadruplex structures from single-strand DNA (Han and Rangan et al., 2000). In this respect, the role of TMPyP4 is very analogous to the β-subunit of the Oxytricha telomere binding protein, altering the dimerization kinetics from second to first order.

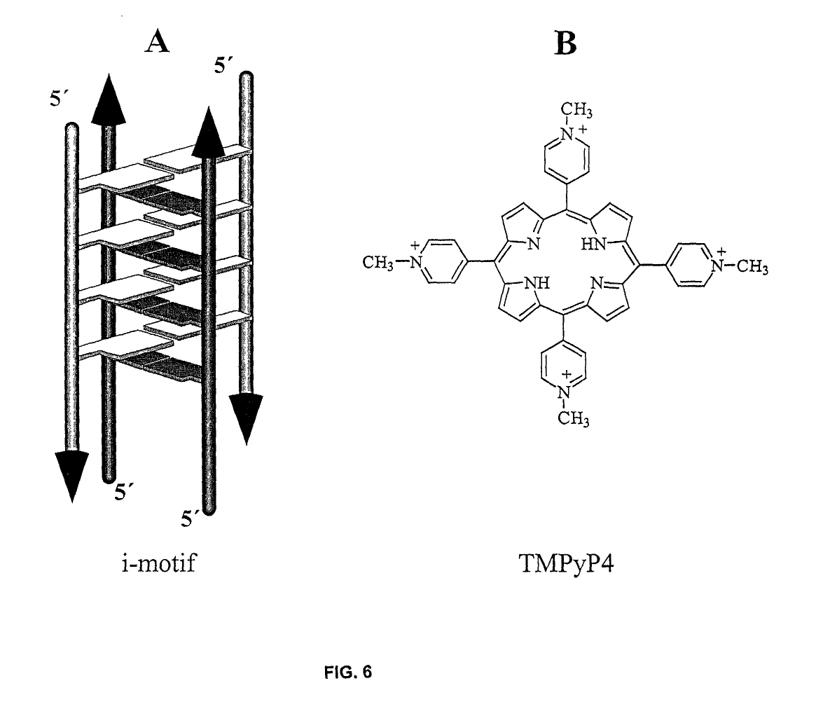

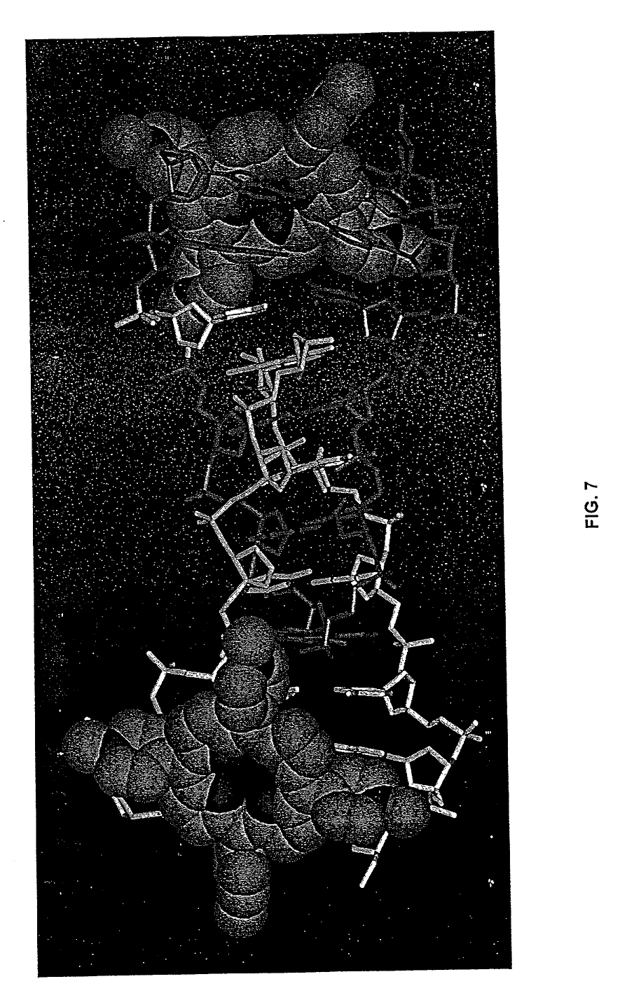

The pyrimidine-rich strands of the NHE can also adopt a secondary DNA structure called an i-motif. I-motifs are four-stranded structures (Gehring et al., 1993; Leroy et al., 1993; Leroy et al., 1994) in which cytidine/cytidine pairs are interdigitated, as shown in FIG. 6. TMPyP4 has also been demonstrated to interact with the i-motif structure, as shown in FIG. 7 (Fedoroff et al., 2000).

IV. Telomerase

An extensive discussion of telomerase may be found in U.S. Pat. Nos. 6,087,493 and 6,156,763, both of which are herein incorporated by reference. Telomerase is a ribonucleoprotein enzyme that synthesizes one strand of the telomeric DNA using as a template a sequence contained within the RNA component of the enzyme. The ends of chromosomes have specialized sequences, termed telomeres, comprising tandem repeats of simple DNA sequences which in humans is 5′-TTAGGG (Blackburn, 1991; Blackburn et al., 1995). Apart from protecting ends of chromosomes telomeres have several other functions, the most important of which appear to be associated with replication, regulating the cell cycle clock and ageing (Counter et al., 1992). Progressive rounds of cell division shorten telomeres by 50-200 nucleotides per round. Almost all tumor cells have shortened telomeres, which are maintained at a constant length (Allshire et al., 1988; Harley et al., 1990; Harley et al., 1994) and are associated with chromosome instability and cell immortalization.

With regard to human cells and tissues telomerase activity has been identified in immortal cell lines and in ovarian carcinoma but has not been detected at biologically significant levels (that are required to maintain telomere length over many cell divisions) in mortal cell strains or in normal non-germline tissues (Counter et al., 1992). These observations suggest telomerase activity is directly involved in telomere maintenance, linking this enzyme to cell immortality.

As described above, the immortalization of cells involves the activation of telomerase. More specifically, the connection between telomerase activity and the ability of many tumor cell lines, including skin, connective tissue, adipose, breast, lung, stomach, pancreas, ovary, cervix, uterus, kidney, bladder, colon, prostate, central nervous system (CNS), retina and blood tumor cell lines, to remain immortal has been demonstrated by analysis of telomerase activity (Kim et al., 1994). This analysis, supplemented by data that indicates that the shortening of telomere length can provide the signal for replicative senescence in normal cells, demonstrates that inhibition of telomerase activity can be an effective anti-cancer therapy. Thus, telomerase activity can prevent the onset of otherwise normal replicative senescence by preventing the normal reduction of telomere length and the concurrent cessation of cell replication that occurs in normal somatic cells after many cell divisions. In cancer cells, where the malignant phenotype is due to loss of cell cycle or growth controls or other genetic damage, an absence of telomerase activity permits the loss of telomeric DNA during cell division, resulting in chromosomal rearrangements and aberrations that lead ultimately to cell death. However, in cancer cells having telomerase activity, telomeric DNA is not lost during cell division, thereby allowing the cancer cells to become immortal, leading to a terminal prognosis for the patient.

Methods for detecting telomerase activity, as well as for identifying compounds that regulate or affect telomerase activity, together with methods for therapy and diagnosis of cellular senescence and immortalization by controlling telomere length and telomerase activity, have also been described elsewhere.

Human telomeres form structures known as G-quadruplexes. Human telomeres contain numerous repeats of the sequence TTAGGG, exhibiting an enhancement of G and T residues and a paucity of A residues. Intramolecular G-quadruplex DNA may be designed by generating a sequence of human telomere repeats. The G tetrad consists of four G bases hydrogen bonded in Hoogsteen fashion symmetrically disposed about a central axis.

G-rich DNA is known to assume highly stable structures formed by Hoogsteen base pairs between guanine residues (Williamson, 1994; Nadel et al., 1995). These structures, known as G-quadruplexes, are stabilized in the presence of K+ and may have biological roles that are yet to be determined (Henderson et al., 1987; Hardin et al., 1997; Williamson et al., 1989) One particular region of the genome where these structures may play a significant biological role is at the ends of chromosomes where G-rich DNA is normally found (e.g., TTAGGG and TTGGGG tandem repeats in human cells and ciliate Tetrahymena, respectively) (Henderson et al., 1987; Blackburn and Greider, 1995; Sundquist and Heaphy, 1993). In addition, a number of genes containing G-rich DNA have been identified recently, and it has been proposed that the G-rich regions within these genes may regulate gene expression by forming G-quadruplex structures (Sen and Gilbert, 1988; Hommond-Kosack et al, 1993; Murchie and Lilley, 1992; Simonsson et al, 1998). One potential biologically relevant role of G-quadruplex DNA is as a barrier to DNA synthesis (Howell et al., 1996). This barrier has been thoroughly investigated and has been found to be K+ dependent (Woodword et al., 1994). This observation strongly suggests that the formation of G-quadruplex species is responsible for the observed effect on DNA synthesis (Weitzmann et al., 1996).

V. Models for Identification of G-Quadruplex Interactive Compounds

The present invention is based in part on the notion that extended chromophores might thread, and thereby stabilize, quadruplexes. 5,10,15,20-tetra-(N-methyl-4-pyridyl)porphine, TMPyP4, has previously been determined to be approximately the appropriate size to stack with the G-tetrads of quadruplex DNA. Similarly, the thiaporphyrins, selenaporphyrins, and carotenoid porphyrins of the present invention are also of the appropriate size to stack with the G-tetrads of quadruplex DNA.

The solution structure of human telomeric G-quadruplex DNA, d(AG3[T2AG3]3) has been determined (Wang and Patel, 1993). It is an intramolecular fold-over structure that is stabilized by three guanine tetrads, stacked at its center. The interaction of TMPyP4 with duplex DNA has been the subject of much work. The X-ray crystal structure of this porphyrin with a short duplex has been solved (Lipscomb et al., 1996). This structure has two notable features: the porphyrin only fits halfway into the duplex and there is extensive disruption of the bases adjacent to the intercalation site.

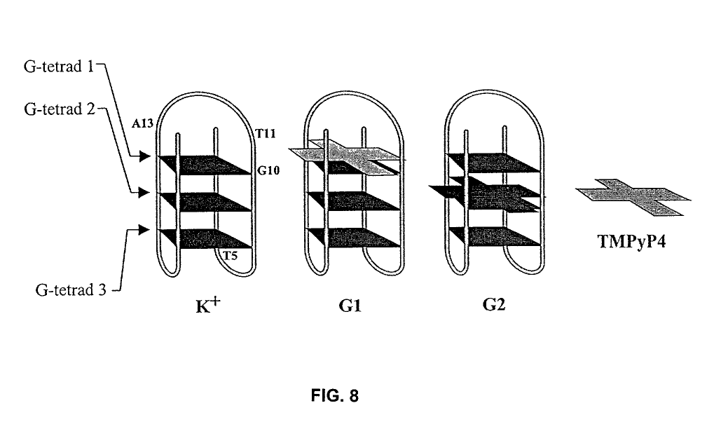

Investigations with quadruplexes indicated that the favored site for TMPyP4 is bound in the loop regions of quadruplexes and stacked externally on the tetrads rather than opening up a true intercalation site. Haq et al. have proposed a purely intercalation model in which the porphyrins bind between the G-tetrads (Haq et al., 1999). This postulate is based upon molecular modeling and stoichiometry measurement. The inventors have reinvestigated the molecular modeling and demonstrated that the external binding mode (shown a structure G1 in FIG. 8) is more favored over the intra-tetrad model (shown as structure G2 in FIG. 8). Photocleavage results do not reveal an intra-tetrad binding, although it is possible, the photocleavage is quenched with this mode of binding. These results and those of Haq et al. can be reconciled if an externally stacked favored site for binding as demonstrated by photocleavage and a less favored intra-tetrad site, which is invisible to photocleavage by TMPyP4, are assumed.

The complex formed with TMPyP4 stabilized quadruplex to thermal denaturation. The solution structure for a twenty-two-base oligonucleotide based on the human telomere sequence, d(AG3[T2AG3]3), (SEQ ID NO: 9), consists of a single looped strand which is stabilized by a core of stacked G-tetrads. The quadruplex can accommodate porphyrins above and below the tetrads at the core of the complex with very little distortion. The porphyrins lie stacked on the tetrads and can be orientated so that the positively-charged groups were directed into the grooves towards the sugar-phosphate back bone.

The present invention achieves the net inhibition of telomerase by targeting its substrate, the telomere. A rational, structure-based approach to the design of telomere interactive agents was employed by considering unique nucleic acid secondary structures associated with the telomerase reaction cycle. One such structure is the G-quadruplex formed by folding of the single stranded G-rich overhang produced by telomerase activity. The template region of the telomerase RNA has only 1.5 copies of the complementary sequence (3′-CAAUCCCAAUC-5′, (SEQ ID NO: 8)) so after each extension, the end of the DNA must be translocated back to the beginning of the of the coding region prior to the next extension (Blackburn, 1991). Translocation occurs without consumption of high energy cofactors. Other work has shown that potassium ions stabilize the quadruplex and that high concentrations of potassium inhibit telomerase (Zahler et al., 1991). Furthermore, the inventors have shown that there is an equilibrium between the DNA:RNA heteroduplex and the G-quadruplex that lies in favor of G-quadruplex formation (Salazar et al., 1996). These observations point to the involvement of G-quadruplex formation in dissociating the primer from the telomerase or RNA template and possibly providing the driving force for the translocation reaction. Thus the inventors hypothesized that the G-quadruplex would be a viable target for drug design as first suggested by Blackburn (Blackburn, 1991).

An objective of the inventors' studies was to identify effective G-quadruplex interactive agents (with significant concentration differences between telomerase inhibition and the cytotoxic effects). Herein the inventors describe the inhibition of telomerase by various thiaporphyrins, selenaporphyrins, and carotenoid porphyrins.

Several methods for identifying classes of G-quadruplex interactive agents may be employed. One method involves identifying compounds whose three-dimensional structure is complementary to that of the G-quadruplex structure. These structures may be determined by a variety of techniques including molecular mechanics calculations, molecular dynamics calculations, constrained molecular dynamics calculations in which the constraints are determined by NMR spectroscopy, distance geometry in which the distance matrix is partially determined by NMR spectroscopy, x-ray diffraction, or neutron diffraction techniques. In the case of all these techniques, the structure can be determined in the presence or absence of any ligands known to interact with G-quadruplex structures.

Complementary is understood to mean the existence of a chemical attraction between the G-quadruplex interactive agent and the G-quadruplex. The chemical interaction may be due to one or a variety of favorable interactions, including ionic, ion-dipole, dipole-dipole, van der Waals, charge-transfer, and hydrophobic interactions. Each of these type of interactions, alone or together, may be determined by existing computer programs using as inputs the structure of the compound, the structure of the G-quadruplex, and the relative orientation of the two. The relative orientation of the two can be determined manually, by visual inspection, or by using other computer programs which generate a large number of possible orientations.

Another method for identifying G-quadruplex interactive compounds that may inhibit telomerase involves use of techniques such as UV/VIS spectroscopy, polarimetry, CD or ORD spectroscopy, IR or Raman spectroscopy, NMR spectroscopy, fluorescence spectroscopy, HPLC, gel electrophoresis, capillary gel electrophoresis, dialysis, refractometry, conductometry, atomic force microscopy, polarography, dielectometry, calorimetry, solubility, EPR or mass spectroscopy. The application of these methods can be direct, in which the G-quadruplex interactive compound's interaction with the G-quadruplex is measured directly, or it can be indirect, in which a particular G-quadruplex interactive agent having a useful spectroscopic property is used as a probe for the ability of other compounds to bind to the G-quadruplex; for example, by displacement or by fluorescence quenching.

VI. General Design and Synthesis of Porphyrins

There are three approaches to the design of porphyrin and core-subsitited porphyrin telomerase inhibitors. The term “core-substituted” means that one or more of the core nitorgen atoms of the porphyrin have been replaced with another atom, such as sulphur or selinium.

(i) Simple Porphyrins

The tetra(N-methyl-4-pyridyl)porphyrin skeleton presents an appealingly elegant and simple structure. The effects of a number of cationic and uncharged hydrophilic groups on the activity against telomerase are contemplated to provide additional support for the design of porphyrin compounds that act effectively as telomerase inhibitors. While the isolated enzyme assay has shown that optimally four positive charges are required on the porphyrin, it is possible that these may pose cellular uptake problems in vitro and absorption problems in vivo. Synthesis may be adapted to prepare 2+ or 3+ analogs which may address these problems.

(ii) Exploitation of the Different Groove Widths

Selectivity in binding can be achieved by the use of groove-binding ligands with strong steric preferences. There is precedent in the literature for achieving selectively between duplex and triplex binding by attaching groove binding ligands at the ends of an appropriately sized aromatic chromophore (Haq et al., 1996). The porphyrin skeleton will be used to build such compounds using ligands such as Hoechst compound 33258 (which in duplex selects for the wide minor groove of GC-rich duplex) and netropsin (which on duplex selects for the narrow minor groove of AT4 sequences). All of these ligands are available either commercially or by standard syntheses and may be attached to the porphyrins by amide, ester, ether or similar linkages can be prepared with up to 4 ligand groups.

(iii) Design of Meso Substituents Tailored to the Grooves of G-Quadruplex

Using a model of the complex between ATTTTTAGGGTTAGGGTTAGGG (SEQ ID NO: 2) and tetra(N-methyl-4-pyridyl)porphyrin, the space in the groove was mapped out to determine what structures could be devised that would deliver a hydrogen bond accepting group to an appropriate location (distance and angle of approach) proximal to the guanine N(2)H above or below the intercalation site occupied by the porphyrin. This result can be used to design new meso substituents for novel porphyrins. The two positions adjacent to the bond to the porphyrin must be either CH or N to allow the compound to achieve planarity for intercalation. One may use a molecular “scaffold” to attach a hydrogen bond acceptor, a. From this structure a variety of compounds based on substituted quinolines and pyridines can be envisioned.

(iv) Other Uses of G-quadruplex Specific Porphyrins

There are two additional ways in which G-quadruplex selective porphyrins and cpore-substituted porphyrins may be used in the development of therapeutics:

(a) G-Quadruplex Cleavage

A compound with the porphyrin linked to a DNA cleavage agent such as iron-EDTA is expected to selectively destroy G-quadruplex thus rendering telomerase activity futile.

(b) Alkylation

The porphyrin may be attached to a group which covalently links to the quadruplex thus rendering the quadruplex more stable and either interfering with telomerase function or blocking the production of telomeric duplex by DNA polymerase.

VII. Structure Activity Relationships

Cationic thiaporphyrins, selenaporphyrins, and carotenoid porphyrins have been identified which are effective inhibitors of c-myc and telomerase. From data pertaining to a wide range of analogues a basic SAR can be determined, viz.: the face of the porphyrins must be available for stacking; the positively charged substituents are important but may be interchanged and combined with hydrogen bonding groups; substitution is only tolerated on the meso positions of the porphyrins and the size of the substituents should to be matched to the width of the grooves in which they lie. These factors are all consistent with a model in which the porphyrins stack externally on the G-tetrads of quadruplex, placing the meso substituents in each of the four grooves.

The cationic porphyrins represent a very promising class of compounds for the development of clinical c-myc and telomerase inhibitors. For a compound to be useful it must have a significant therapeutic window between its activity against telomerase and the onset of cytotoxic effects. The cationic porphyrins of the present invention have a number of properties that render them attractive candidates for development as c-myc and telomerase inhibitors for the treatment of patients with cancer. The data presented herein lay the foundations for a program of drug development to achieve the dual aims of efficacy and selectivity in vivo.

Groups of compounds were selected to determine the effects of steric and electronic variations on the TMPyP4 structure: coordinated metal ion, number of charges on the meso substituents, disposition of charges around the porphyrin ring, position of the charged group on the pyridyl and quinolyl substituents, bulk of substituents, effects of different quaternizing groups (Me, Et, HO-Et, CH₂OAc). A wide range of natural porphyrins and their close analogues was also investigated.

VIII. Design and Synthesis of Thiaporphyrins

The structure activity relationship (SAR) and modeling data described above make an initial assumption that the disclosed porphyrins likely interact with G-quadruplex. This provides a basis for the design of new compounds with both increased binding constants and selectivity for quadruplex, and thus increased activity against telomerase. DNA quadruplex presents a unique structure with significant differences from duplex DNA. Firstly, steric accessibility of the grooves: one wide, one narrow and two medium width grooves. Secondly, the hydrogen bonding potential of groups in the base of the grooves. The symmetry of the G-tetrads results in all four grooves having the same hydrogen bonding group: one guanine-N(2)H per tetrad in each groove; significantly less hydrogen bonding potential than found in duplex.

While various approaches to synthesizing core modified porphyrins with neutral or electron donating groups are known, methods of synthesizing di- and mono-thiaporphyrins with cationic electron withdrawing groups were not previously known.

According to previous studies (Latos-Grazynski et al., 1991; Latos-Grazynski et al., 1995) of core modified porphyrins with neutral or electron donating groups, the replacement of the core nitrogen atoms with sulfur has interesting effects on both aromatic character and central core shape. X-ray crystal structure of thiaporphyrins reveals a non-planar, distorted geometry. It is important that these molecules might bind to intramolecular G-quadruplex structures where the space between the stacked tetrads and the fold-over loops form appropriately shaped cavities. Furthermore, the variation of the central cavity size may affect the flow of ions through the porphyrin core.

A wide range of TMPyP4 analoges have been synthesized and assayed against telomerase. However, they have the potential problem of photo-induced skin toxicity, which may affect their clinical use. In order to overcome this disadvantage, one of the most interesting and promising approaches involves performing specific atom replacements at the porphyrin core. The analogues of pyridyl containing normal porphyrins have the potential problem of photo-induced skin toxicity. Recent reports show that the core modified porphyrins containing thiophene have no photoinduced skin toxicity (Ziolkowski et al., 1995; Marcinokowska et al., 1997).

An example of a core modified porphyrin is the recently synthesized 21-thiaporphyrin analog 10,15-bis(2-methoxy-(4-sulphophenyl)-21-thiaporphyrin (STSP) (Ziolkowski et al., 1995; Marcinokowska et al., 1997). This new PDT (Photodynamic Therapy) agent has been shown to be a highly effective photosensitization agent both in vitro and in vivo. In contrast, STSP was reported to show no photoinduced skin damage. The reason for the lack of skin toxicity for STSP is not entirely clear. It may simply be that STSP does not accumulate well in skin tissue. It does, however, offer the possibility that these types of mixed porphyrins will have fewer clinical side effects.

In general, introduction of heteroatoms in the core alters the electronic structure of the porphyrin macrocycle, which in turn leads to many interesting properties in spectral and electrochemical aspects. Electrochemical and theoretical calculations have shown that the chalcogen atoms drain π electrons from the porphyrin ring and take part in direct bonding interactions across the ring. A comparison of spectral data of normal pyrrole-containing porphyrins with that of core modified porphyrins reveals the following observations. The absorption spectra of thiaporphyrins is more red-shifted (absorbs in the higher wavelength region) than the normal porphyrins. The emission properties of these porphyrins were studied with the fluorescence spectroscopy. The data on the emission spectra revealed that substitution of the core with the heavier atoms quenches the fluorescence. The data suggest that there is a decrease in the quantum yield as we go from tetraphenyl porphyrin to tetraphenyl monothia porphyrin and a further decrease in tetraphenyl dithia porphyrin.

The thiaporphyrins may be coordinated to a metal. A range of metal complexes (transition metals and lanthamides) can be prepared to identify those metals that confer the best combination of low photosentization, strong telomerase inhibition, and low cytotoxicity on the porphyrin. The preferred metal ions may be used in the more elaborate porphyrin conjugates.

A. Previous Approaches to Thiaporphyrin Synthesis

1. Tetraphenyl-21,23-dithiaporphyrins

The synthesis of symmetrically as well as unsymmetrically substituted tetraphenyl-21,23-dithiaporphyrins (S2TPP) was originally developed by A. Ulman and co-workers (Ulman et al., 1975; Ulman et al., 1978a; Ulman et al., 1978b; Ulman and Frolow et al., 1979; Ulman and Manassen, 1979; Ulman et al., 1987). The reaction of 2,5-dilithiothiophene with substituted benzaldehydes was used for the preparation of substituted 2,5-bis(phenylhydroxymethyl)thiophens (Scheme I). Symmetrically substituted S2TPP molecules result from the reaction of these dialcohols with equimolar quantities of pyrrole using an acid as catalyst under air. Only the catalyst/solvent system chloroacetic acid/benzene, chloroacetic acid/toluene, and propionic acid gave reasonable yields.

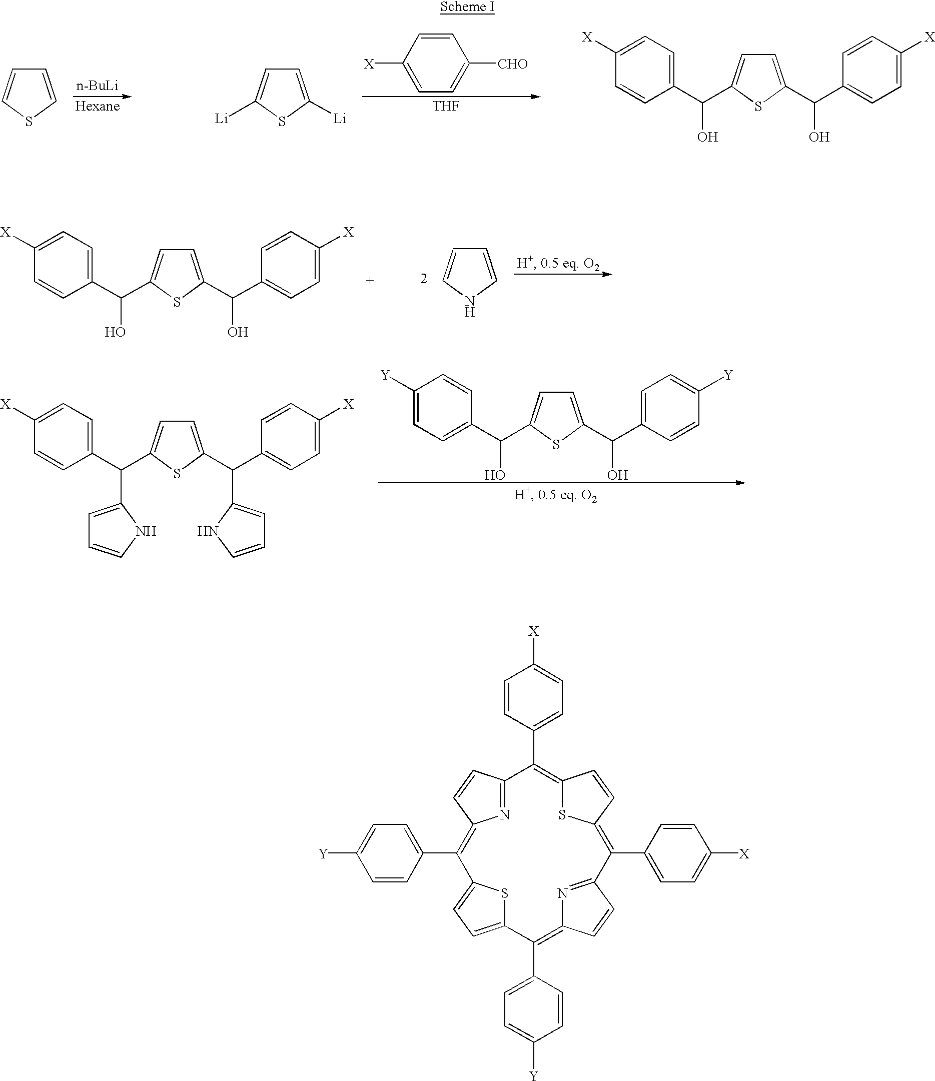

A modification to this synthesis was developed by Lindsey (Lindsey et al., 1989). This method relied on the formation of porphyrinogen as an intermediate in the porphyrin synthesis (Scheme II). The reaction was carried out under inert conditions in dichloromethane or chloroform for 1 h, using boron trifluoride or trifluoroacetic acid as catalyst to establish an equilibrium with tetraphenylporphyrinogen, followed by the oxidation by excess DDQ for a further hour at reflux. The advantage of this method is that it allows the formation of porphyrins from sensitive aldehydes in higher yields with more facile purification. A drawback, however, is the need for higher dilution conditions, which means that the reaction is not amenable to scale-up.

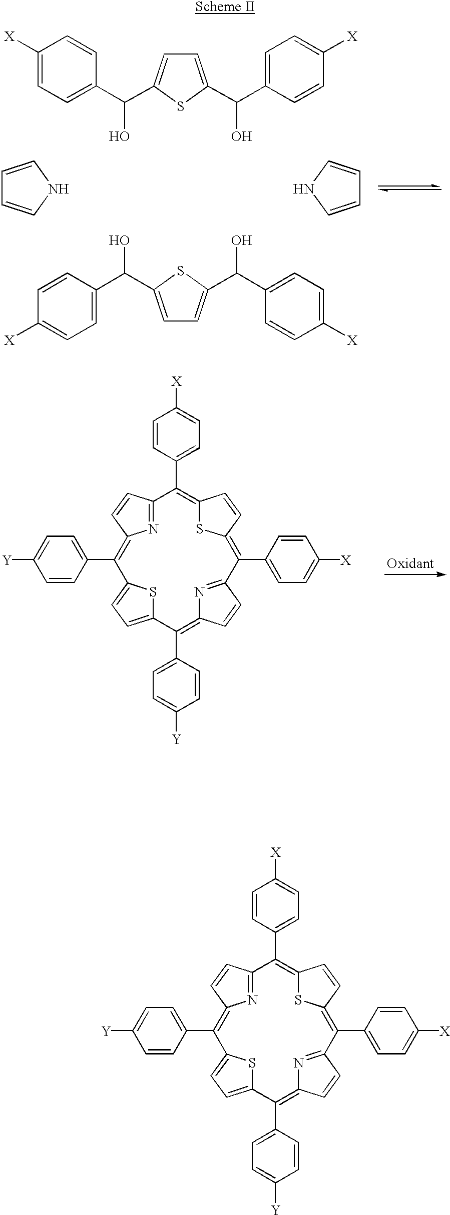

2. Tetraphenyl-21-monothiaporphyrins

The synthesis of tetraphenyl-21-monothiaporphyrins was reported by Latos-Grazynski (Latos-Grazynski et al., 1991). This route to the monothiaporphyrins allows for the introduction of unsymmetrical substituents on the porphyrin periphery (Scheme III).

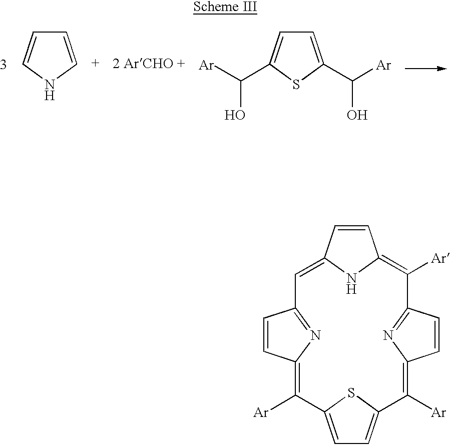

The improved synthesis of monothiaporphyrins was developed by Srinivasan (Srinivasan et al., 1997) using a variety of meso substituted dipyrromethanes with the corresponding thiophen dialcohols (Scheme IV).

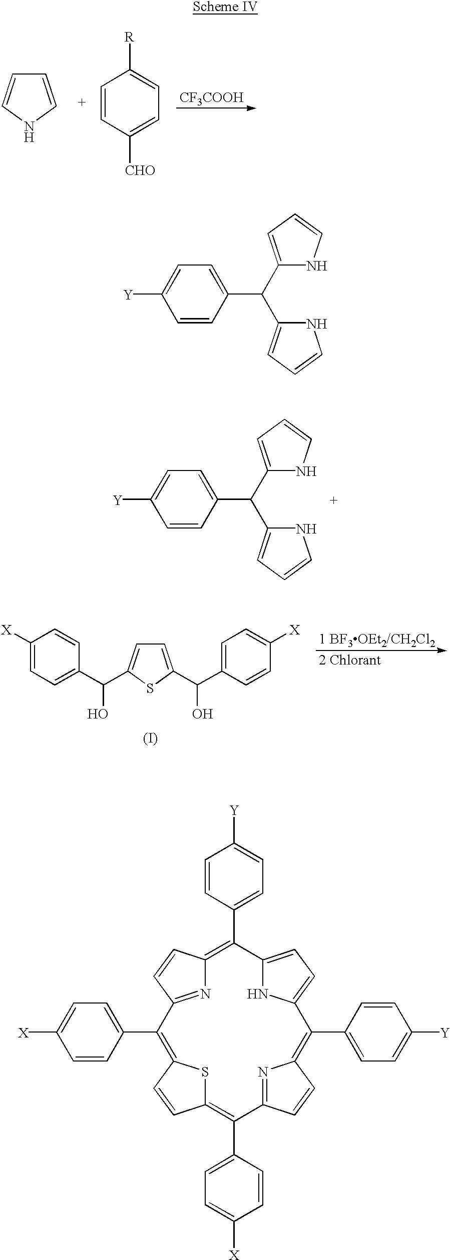

3. Problems with the Synthesis of Cationic Thiaporphyrin

The intermediate thiophen dialcohols (I) were also applied to the formation of thiaporphyrins with cationic pyridyl or quinolyl group. The typical methodologies for the synthesis of mono- or di-thiaporphyrins were attempted under varying reaction conditions. However, no formation of thiaporphyrins bearing pyridyl or quinolyl ring could be substantiated. The Lindsey method was particularly problematic. The main reason for the failure of formation of cationic thiaporphyrins was the solubility of dialcohol in dichloromethane or chloroform and the duration of the oxidation reaction. It was supposed that the oxidation under air was inefficient when the substrates were thiaporphyrins bearing electron withdrawing groups. Consequently Ulman's original method was mainly applied in the synthetic procedures of the present invention.

B. New Methodology for the Synthesis of Cationic Thiaporphyrins

The condensation was attempted in several solvent systems. Because the thiophen dialcohols with pyridyl or quinlyl groups are more polar than those with neutral or electron donating groups, they were not soluble in either dichloromethane or chloroform. One acceptable solvent used was propionic acid, in which the dialcohol can dissolve at reflux temperature. The other advantage of this method was that propionic acid was also the catalyst.

Condensation and oxidation in propionic acid under air for 1 h are the reaction condition normally sufficient for standard porphyrins. However, almost no products formed when these reaction conditions were applied to the synthesis of thiaporphyrins bearing pyridyl or quinolyl rings. Reasonable yields were isolated when the duration was 4 h or more at reflux, presumably allowing sufficient time for oxidation.

Symmetrically substituted monothiaporphyrin compounds were isolated when the reaction duration was extended to 4 hours via condensation of 3-pyridyl substituted dialcohol with pyrrole and 3-pyridinecarboxaldehyde in propionic acid.

The 3-pyridyl substituted tetrahydrotripyrrin, an unsymmetically substituted monothiaporphyrin, was first synthesized by reacting the dialcohol with excess pyrrole in propionic acid under argon (Lee et al., 1997). The tetrahydrotripyrrin was then condensed with pyrrole and 4-pyridinecarboxaldehyde to give the monothiaporphyrin in good yield (Scheme V).

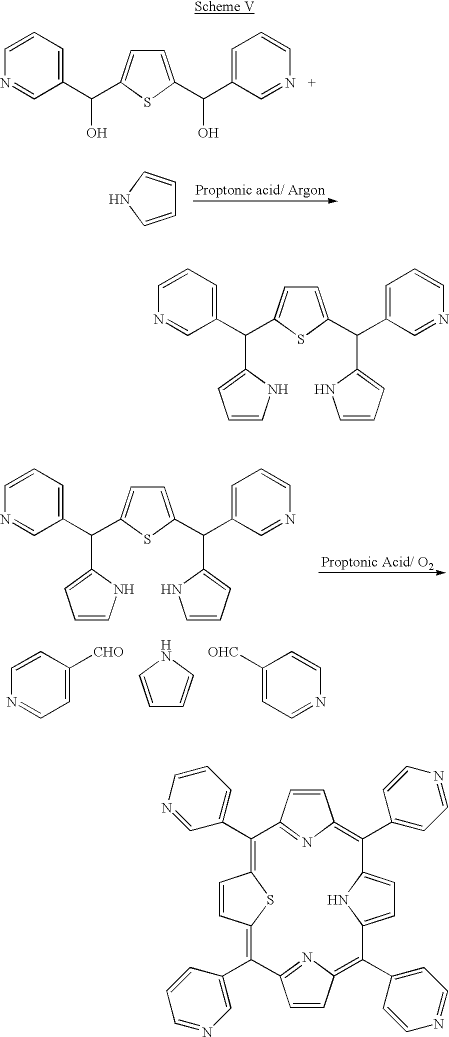

C. Synthesis of Various Thiaporphyrins

Structures for the thiaporphyrins discussed in this section are shown in Table 1.

TABLE 1

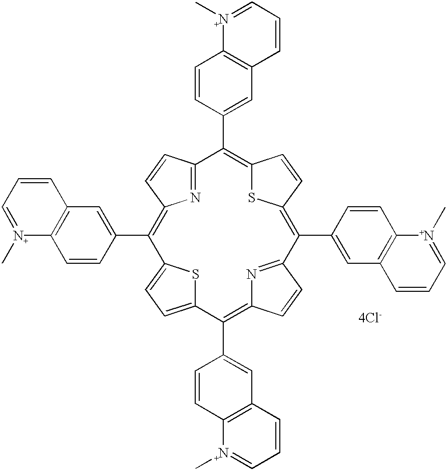

C₆₀H₄₄Cl₄N₆S₂ = 1054.9842
DL85

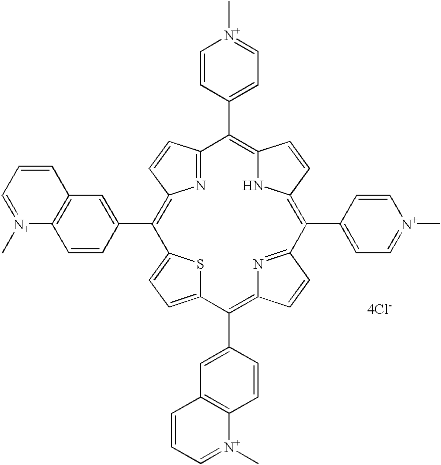

C₅₂H₄₁Cl₄N₇S = 937.82
DL86

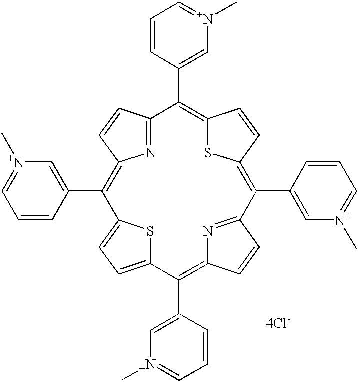

C₄₄H₃₆Cl₄N₆S₂ = 854.74
DL95

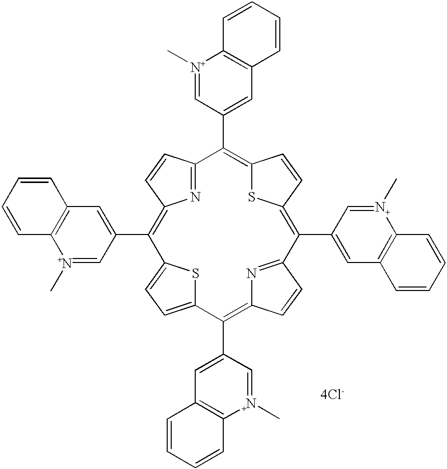

C₆₀H₄₄Cl₄N₆S₂ = 1054.9
DL96

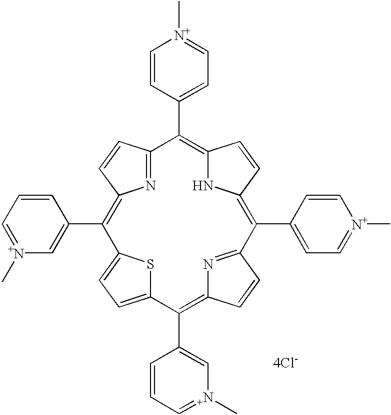

C₄₄H₃₇Cl₄N₇S = 837.6
DL97

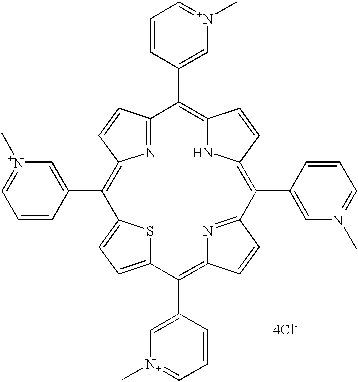

C₄₄H₃₇Cl₄N₇S = 837.69
DL98

Porphyrins that were not commercially available were synthesized by variations on established methods. The standard porphyrin synthesis is the acid catalyzed condensation of pyrrole with an heteroaryl aldehydes or 2,5-bis(α-hydroxy-α-pyridylmethyl)thiophene to give the novel porphyrins with mixed meso substituents. Quaternization of the free-bases of pyridyl- and quinolyl-substituted porphyrins was accomplished by reaction with alkyliodides in chloroform or a mixture of chloroform and nitromethane, The salts precitated and were converted to the chloride form by ion exchange to give the final cationic porphyrins. The synthesis of various thiaporphyrins is provided below.

To the right of several of the chemical names listed below appears a paranthetical designation, such as “(T1).” This is used as a shorthand designation of the compound. Preceeding references to that designation, such as “compound (T1)” reference the compound associated with that designation.

1. Preparation of 5,10,15,20-tetra(N-methyl-6-quinolyl)-21,23-dithiaporphyrin chloride (DL85)

a) Preparation of 2,5-bis(4-acetamidophenylhydroxymethyl)thiophene (T1). To a three-necked, round bottomed flask flushed with argon was added 80 mL of anhydrous hexane, 7.6 mL (0.05 mol) of TMEDA and 20 mL (2.5M in hexane) (0.05 mol) of n-butyllithium, 1.61 μL (0.02 mol) of thiophene was then added at room temperature, the mixture was refluxed for 1 h. After cooling to room temperature, the suspension formed was slowly transferred dropwise via needle to a degassed solution of 6.52 g (0.04 mol) of 4-acetamidobenzaldehyde in 200 mL of anhydrous THF in an ice-bath. After the addition was completed, the mixture was allowed to warm to room temperature and stirred for further 30 min. 20 mL of methanol and ice-cold NH4Cl was added separately with stirring. The phases were separated and the water layer was extracted with chloroform. The organic layers were combined, washed with water and dried over Na2SO4. After removal of solvent, the residue was purified by chromatography on silica gel using chloroform-methanol (8:1) as eluent giving (T1) (5%). 1HNMR (DMSO-d₆) δ 9.89 (s, 2H), 7.48 (d, J=8.41 Hz, 4H), 7.25 (d, J=8.24 Hz, 4H), 6.59 (d, 2H), 6.01 (d, 2H), 5.73 (d, 2H), 2.00 (s, 6H); 13CNMR(DMSO-d₆) δ 168.1, 149.8, 139.8, 138.2, 126.3, 122.8, 118.7, 70.2, 24.0; MS (CI) 411 (M+H).

b) Preparation of 5,10,15,20-tetra(4-acetamidophenyl)-21,23-dithia-porphyrin (T2). A mixture of 1.32 g (3.21 mmol) of compound (Ti) and 0.215 g (3.21 mmol) of pyrrole was dissolved in 500 mL of propionic acid. The mixture was heated to reflux for 1 h. After cooling to room temperature, the solvent was evaporated to dryness under high vacuum. The residue was purified by chromatography on silica gel using chloroform-methanol (8:1 to 6:1) as eluent gave (T2) (5%). 1HNMR (CDCl3/CD3OD) δ 9.44 (s, 4H), 8.40 (s, 4H), 7.90 (d, J=8.4 Hz, 8H), 7.75 (d, J=8.3 Hz, 8H), 2.03 (s, 12H); MS (CI) 877(M).

c) Preparation of 5,10,15,20-tetra(4-aminophenyl)-21,23-dithia-porphyrin (T3). 215 mg of crude compound (T2) was dissolved in 25 mL of TFA and 30 mL of conc. HCl was added at room temperature. The resulting mixture was heated at 80-85° C. for 24 h, then cooled to 0° C. It was diluted with 10 mL of water, neutralized with 1N NaOH to pH 8-9, extracted with chloroform, and then the organic layer was dried over Na2SO4. After removal of solvent, the residue was purified by chromatography on silica gel using chloroform-methanol (8:1) as eluent gave (T3) (80%). HRMS (CI) (M+H). calcd 709.2208, obsd 709.2208, C44H32N6S2.

d) Preparation of 5,10,15,20-tetra(6-quinolyl)-21,23-dithiaporphyrin (T4). To a stirred mixture of 111 mg (0.9 mmol) of nitrobenzene, 138 mg (1.5 mmol) of glycerol and 53 mg (0.075 mmol) of compound (T3) heated at 120° C., sulphuric acid was added in portion, the resulting mixture then was maintained at 140° C. for 5 h, 2N NaOH was added until pH 9-10. The mixture was extracted with CHCl3, and the organic layer was dried over Na2SO4. After removal of solvent, the residue was separated with PTLC (chloroform-methanol 95:5) to give (T4) (23.7%). 1HNMR (CDCl3) δ 9.69 (s, 4H), 9.15 (d, 4H), 8.68-8.64 (br d, 12H), 8.52 (d, 4H), 8.42 (d, 4H), 7.62 (dd, J=4.23 Hz, 4H); HRMS (CI) (M+H) calcd 853.2208, obsd 853.2224, C56H33N6S2.

e) Preparation of DL85. 13.2 mg (0.155 mmol) of compound (T4) was dissolved in 4.0 mL of chloroform and diluted with 3.0 mL of nitromethane. 3.0 mL of iodomethane was added and the mixture was heated at reflux under argon for 6 h and then stirred overnight. After removal of solvent to dryness, 5.0 mL of water was added to the residue and treated with 2.0 g of Dowex 1×2-200 anion exchange resin in the chloride form, shaking slowly for 2 h. The resin was filtered off, washed with water, and the filtrate lyophilized to give the chloride salt (70%). The salt could be further purified by chromatography on lipophilic sephadex using methanol as eluent. 1HNMR (DMSO-d₆) δ 9.88-9.80 (m, 8H), 9.63 (br d, 4H), 9.43 (d, 4H), 9.20 (br t, 4H), 9.04 (d, 4H), 8.69 (br s, 4H), 8.42 (m, 4H), 4.95 (s, 12H); HRMS (FAB) (M) calcd 912.3069, obsd 912.3077, C60H44N6S2.

2. Preparation of 5,10,15,20-tetra(N-methyl-3-pyridyl)-21,23-dithiaporphyrin Chloride (DL95)

DL95 was prepared according to Scheme VI. The key intermediate, 2,5-bis(α-hydroxy-α-pyridylmethyl)thiophene, can be synthesized (Ulman et al., 1979; Chadwick et al., 1977) by reacting thiophene with n-butyllithium, then with pyridinecarboxyaldehyde.A detailed description of the steps is provided below.

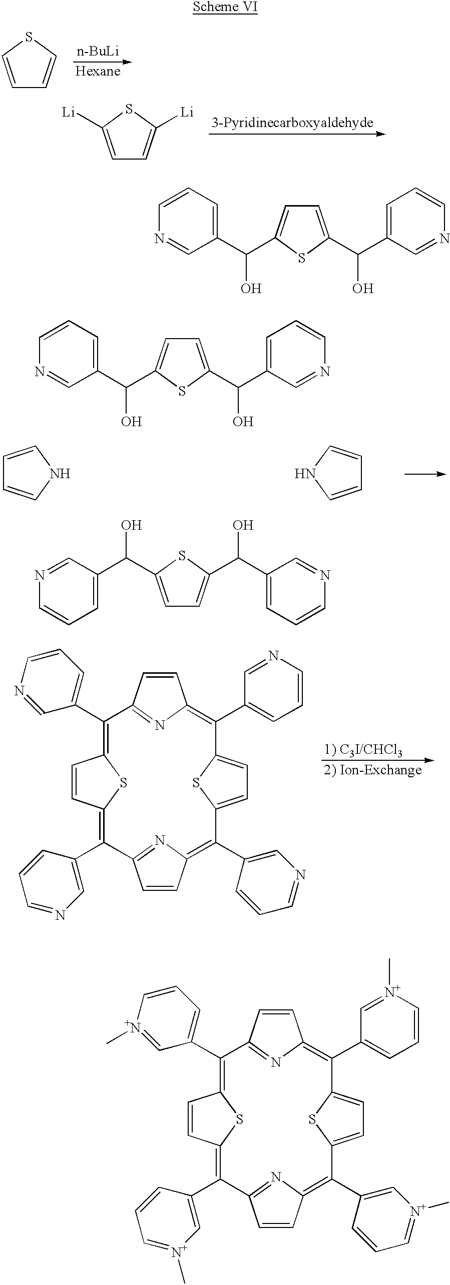

a) Preparation of 2,5-bis(3-pyridylhydroxymethyl)thiophene (T5). According to the same procedure as preparation of compound (T1), reaction of 3-pyridinecarboxaldehyde with 2,5-dilithiothiophene gave (T5) (47.6%). 1HNMR (DMSO-d6) δ 8.57 (br s, 2H), 8.44 (dd, J=4.7 Hz, 2H), 7.75 (br d, 2H), 7.34 (dd, J=4.7 2H), 6.70 (d, 2H), 5.92 (d, 2H); 13CNMR (DMSO-d₆) δ 148.8, 148.6, 148.5, 147.6, 140.0, 133.7, 123.4, 68.6; MS (CI) 299(M+H).

b) Preparation of 5,10,15,20-tetra(3-pyridyl)-21,23-dithiaporphyrin (T6). Prepared from condensation of 1.52 g (5.1 mmol) of compound (T5), 0.34 g (5.1 mmol) of pyrrole in propionic acid with 1 mL of acetic anhydride for 5 h according to general method. Chromatography on silica gel using chloroform-methanol (95:5) as eluent gave (T6) (1.8%). 1HNMR (CDCl3) δ 9.61 (s, 4H), 9.33 (s, 4H), 8.94 (d, 4H), 8.60 (s, 4H), 8.52 (d, 4H), 7.79 (dd, 4H); 13CNMR (CDCl3/CD3OD) δ 156.4, 152.5, 149.0, 148.0, 141.0, 136.2, 135.5, 134.7, 130.0, 123.1; HRMS (CI) (M+H) calcd 653.1582, obsd 653.1583, C40H25N6S2.

c) Preparation of DL95. Prepared from (T6) with iodomethane according to general method, yield 84%. 1HNMR (DMSO-d₆) δ 10.15 (br s, 8H), 9.66 (d, 4H), 9.42 (d, 4H), 9.00 (br s, 4H), 8.70 (t, 4H), 4.75 (s, 12H); HRMS (FAB) (M) calcd 712.2443, obsd 712.2429, C44H36N6S2.

3. Preparation of 5,10-bis(N-methyl-3-pyridyl)-15,20-bis(N-methyl-4-pyridyl)-21-monothiaporphyrin Chloride (DL97)

DL97 was prepared according to Scheme VII. A detailed description of the steps is provided below.

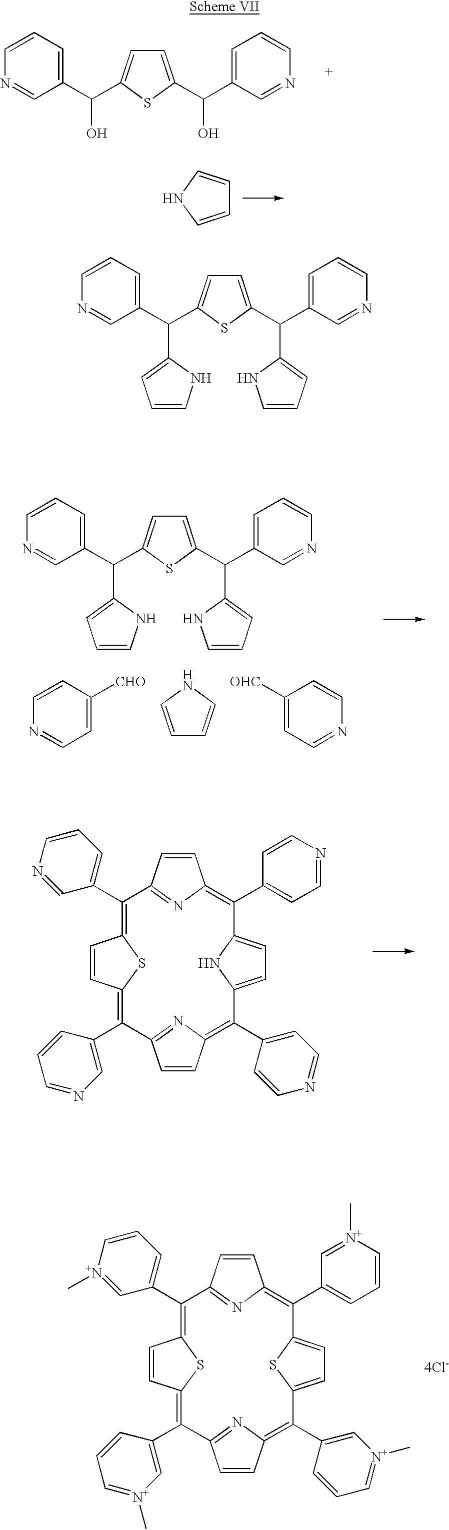

a) Preparation of 2,5-bis[α-(2-pyrryl)-3-pyridylmethyl]thiophene (T7). The mixture of 0 6 g (2.0 mmol) of compound (T5) and 10 mL of pyrrole in propionic acid was refluxed under argon for 3 h, and then evaporated the solvent to dryness. 1HNMR (CDCl3) δ 8.63 (br s, 2H), 8.40 (d, 4H), 7.52 (d, 2H), 7.20 (m, 2H), 6.68 (br s, 2H), 6.57 (s, 2H), 6.09 (q, 2H), 5.82 (br s, 2H), 5.52 (s, 2H); MS (CI) 397(M+H)

b) Preparation of 5,10-bis(3-pyridyl)-15,20-bis(4-pyridyl)-21-monothiaporphyrin (T8). To the solution of compound (T7) formed last step 0.7 mL (10 mmol) of pyrrole and 2.2 g (20 mmol) of 4-pyridinecarboxaldehyde were added with stirring, the resulting mixture was refluxed under air for 2.5 h. After removal of solvent, the residue was separated with column and PTLC (chloroform-methanol 95:5) to give (T8) (7.5%). 1HNMR (CDCl3) δ 9.78 (s, 2H), 9.50 (d, 2H), 9.07-9.04 (m, 6H), 8.96 (d, 2H), 8.70 (d, 2H), 8.61 (d, 2H), 8.57-8.52 (m, 2H), 8.12 (dd, 4H), 7.79 (m, 2H), −2.85 (s, H); HRMS (CI) (M+H) calcd 636.1970, obsd 636.1972, C40H26N7S.

c) Preparation of DL97. Prepared from (T8) with iodomethane according to general method, yield 81%. 1HNMR (DMSO-d₆) δ 10.20 (br s, 2H), 10.10 (br d, 2H), 9.57-9.43 (m, 6H), 9.23 (br d, 2H), 9.10 (br d, 2H), 9.03 (d, 2H), 8.95-8.84 (m, 2H), 8.69 (br s, 4H), 8.29 (m, 2H), 4.73 (br s, 12H), −3.06 (s, H); MS (CI) (M) calcd 695.2831, obsd 695.2866, C44H37N7S.

4) Preparation of 5,10-bis(N-methyl-6-quinolyl)-15,20-bis(N-methyl-4-pyridyl)-21-monothiaporphyrin Chloridem (DL86)

Meso-tetra(6-quinolyl)thiaporphyrin is of interest in the development of a library of core-modified porphyrins by replacement of nitrogen with sulfur as telomerase inhibitors. Because the 6-quinolinecarboxyaldehyde is not commercially available, another approach to make it was investigated. The key intermediate 2,5-bis(α-hydroxy-α-phenylmethyl)thiophene was synthesized by reacting thiophene with n-butyllithium, then with acetamidobenzaldehyde in very poor yield. The resulting compound condensed with pyrrole in propionic acid to give meso-tetra(4-acetamidophenyl)thiaporphyrin. After hydrolysis (Li et al., 1994) in TFA/HCl, the meso-tetra(6-quinolyl)-thiaporphyrin was made by Skraup quinoline synthesis (Yale, 1948; Wahren 1964; Perche, 1972).

DL86 was prepared according to Scheme VIII. A detailed description of the steps is provided below.

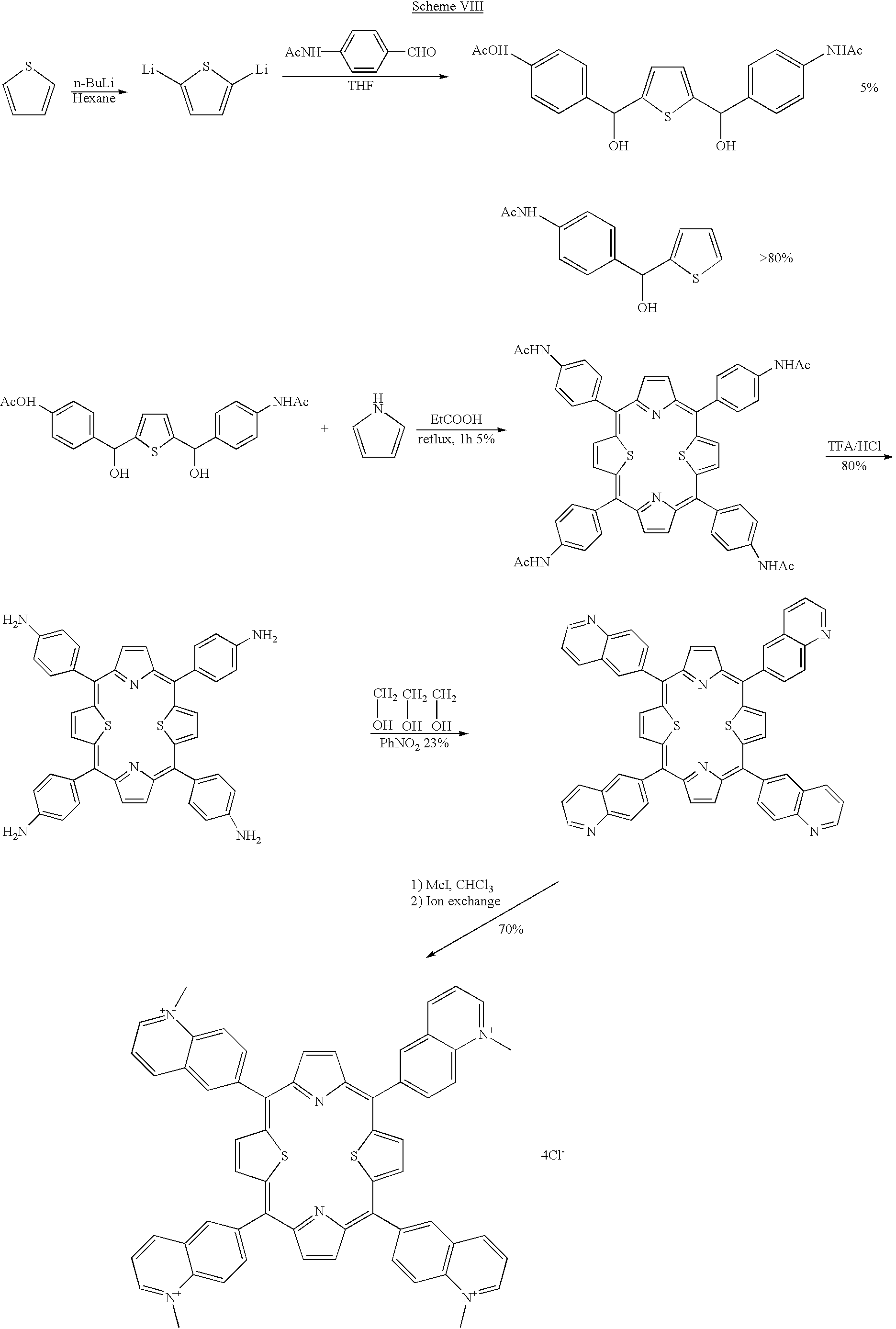

a) Preparation of 5,10-bis(4-acetaniidophenyl)-15,20-bis(4-pyridyl)-21-monothiaporphyrin (T9). A mixture of 1.53 g (3.7 mmol) of compound (T1), 0.8 g (7.4 mmol) of 4-pyridinecarboxaldehyde and 0.78 mL (11.1 mmol) of pyrrole was dissolved in 200mL of propionic acid. The mixture was heated to reflux for 1 h. After cooling to room temperature, the solvent was evaporated to dryness under high vacuum. The residue was purified by chromatography on silica gel using chloroform-methanol (8:1) as eluent gave crude compound (T9). Small amount of sample was further purified by TLC plates for analysis. 1HNMR (DMSO-d₆) δ 10.43 (s, 2H), 9.87 (s, 2H), 9.02 (d, J=5.8 Hz, 4H), 8.99 (s, 2H), 8.72 (d, 2H), 8.52 (d, 2H), 8.24 (d, J=5.8 Hz, 4H), 8.18 (d, J=8.6 Hz, 4H), 8.11 (d, J=8.6 Hz, 4H), 2.07 (s, 6H),-2.98 (s, 1H); MS (CI) 748(M+H).

b) Preparation of 5,10-bis(4-aminophenyl)-15,20-bis(4-pyridyl)-21-monothiaporphyrin (T10). Crude compound (T9) formed in last step was dissolved in 11mL of TFA and 11 mL of conc. HCl was added at room temperature. The resulting mixture was heated at 80-85° C. for 24 h, then cooled to 0° C. It was diluted with 10 mL of water, neutralized with 1N NaOH to pH 8-9, extracted with chloroform, and then the organic layer was dried over Na2SO4. After removal of solvent, the residue was purified by chromatography on silica gel using chloroform-methanol (8:1) as eluent gave (T10) (3.4%). MS (CI) 664(M+H).

c) Preparation of 5,10-bis(6-quinolyl)-15,20-bis(4-pyridyl)-21-monothia-porphyrin (T11). To a stirred mixture of 89.1 mg (0.72 mmol) of nitrobenzene, 110 mg (1.2 mmol) of glycerol and 80 mg (0.12 mmol) of compound (T10) heated at 120° C., 64 mg (0.65 mmol) of sulphuric acid was added in portion, the resulting mixture then was maintained at 140° C. for 5 h, 2N NaOH was added until pH 9-10. The mixture was extracted with CHCl3, and the organic layer was dried over Na2SO4. After removal of solvent, the residue was separated with PTLC (chloroform-methanol 95:5) to give (T11) (16.5%). 1HNMR (CDCl3) δ 9.78 (s, 2H), 9.15 (dd, 2H), 9.02 (d, J=5.3 Hz, 4H), 8.93 (d, 2H), 8.72 (d, J=4.8 Hz, 2H), 8.65-8.62 (m, 4H), 8.57 (d, J=4.8 Hz, 2H) 8.53 (d, 2H), 8.40 (d, 2H), 8.13 (d, J=5.3 Hz, 4H), 7.61 (dd, J=4.3 Hz, 2H), −2.76 (s, 1H); 13CNMR (DMSO-d₆) δ 157.7, 152.6, 151.5, 150.3, 148.2, 147.8, 138.8, 138.1, 136.8, 135.2, 135.1, 134.9, 133.8, 133.0, 131.2, 129.0, 128.7, 127.6, 128.4, 127.7, 122.2, 120.5; MS (CI) 737(M+H).

d) Preparation of DL86. Prepared from (T11) with iodomethane according to general method, yield 65%. 1HNMR (DMSO-d₆) δ 9.92 (s, 2H), 9.87 (d, 2H), 9.64 (d, 2H), 9.55 (d, 4H), 9.42 (s, 2H), 9.30 (d, 2H), 9.20 (d, 2H), 9.06-8.95 (m, 6H), 8.80 (d, 4H), 8.44 (t, 2H), 4.94 (s, 6H), 4.75 (s, 6H), −2.95 (s, 1H); HRMS (FAB) (M+H) calcd 796.3222, obsd 796.3238, C52H42N7S.

5. Preparation of 5,10,15,20-tetra(N-methyl-3-quinolyl)-21,23-dithia-porphyrin Chloride (DL96)

a) Preparation of 2,5-bis(3-quinolylhydroxymethyl)thiophene (T12). To a three-necked, round bottomed flask flushed with argon was added 60mL of anhydrous hexane, 5.7 mL (37.5 mmol) of TMEDA and 15mL (2.5M in hexane) (37.5 mmol) of n-butyllithium, 1.2 mL (15 mmol) of thiophene was then added at room temperature, the mixture was refluxed for 1 h. After cooling to room temperature, the suspension formed was slowly transferred dropwise via needle to a degassed solution of 4.71 g (30 mmol) of 4-acetamidobenzaldehyde in 100 mL of anhydrous THF in an ice-bath. After the addition was completed, the mixture was allowed to warm to room temperature and stirred for further 30 min. 10 mL of methanol and ice-cold NH4Cl was added separately with stirring. Some of product suspended in the solution. After filtration, the crude product was given by 4.18 g. The liquid phases were separated and the water layer was extracted with chloroform. The organic layers were combined, washed with water and dried over Na2SO4. After removal of solvent, the residue was purified by chromatography on silica gel using chloroform-methanol (8:1) as eluent giving (T12) 0.343 g (total yield: 75.7%). 1HNMR (DMSO-d₆) δ 8.89 (s, 2H), 8.33 (s, 2H), 8.02-7.99 (br d, 4H), 7.74 (t, J=6.9 Hz, J=6.6 Hz, 2H), 7.61 (t, J=6.9 Hz, J=6.6 Hz, 2H), 6.82 (d, 2H), 6.51 (br s, 2H), 6.16 (s, 2H); 13CNMR (DMSO-d₆) δ 149.8, 148.9, 146.9, 137.5, 131.9, 129.3, 128.7, 128.2, 127.3, 126.8, 123.8, 68.8; MS (CI) 399(M+H).

b) Preparation of 5,10,15,20-tetra(3-quinolyl)-21,23-dithiaporphyrin (T13). Prepared from condensation of 2.0 g (5 mmol) of compound (12), 0.35 mL (5 mmol) of pyrrole in 250 mL of propionic acid with 1 mL of acetic anhydride for 4 h according to general method. Chromatography on silica gel using chloroform-methanol (95:5) as eluent gave (T13) (6.9%). 1HNMR (CDCl3) δ 9.85 (br s, 4H), 9.76 (s, 4H), 9.03 (br s, 4H), 8.75 (s, 4H), 8.48 (d, 4H), 8.15 (m, 4H), 7.98 (t, 4H), 7.79 (t, 4H); 13CNMR (CDCl3) δ 156.8, 153.7, 148.4, 147.6, 140.2, 135.7, 135.0, 133.8, 130.63, 130.56, 129.7, 128.4, 127.8, 127.1; HRMS (CI) (M+H) calcd 853.2208, obsd 853.2223, C56H33N6S2.

c) Preparation of DL96. Prepared from (T13) with iodomethane according to general method, yield 90%. 1HNMR (DMSO-d₆) δ 10.76 (br d, 4H), 10.26 (br d, 4H), 10.21 (s, 4H), 9.10 (s, 4H), 8.92-8.86 (m, 8H), 8.57 (t, 4H), 8.33 (t, 4H), 5.00 (s, 12H); HRMS (FAB) (M) calcd 912.3069, obsd 912.3072, C60H44N6S2.

6) Preparation of 5,10,15,20-tetra(N-methyl-3-pyridyl)-21-monothia-porphyrin Chloride (DL98)

a) Preparation of 2,5-bis[α-(2-pyrryl)-3-pyridylmethyl]thiophene (T7). The mixture of 0.06 g (0.2 mmol) of compound (T5) and 1.0 mL of pyrrole in propionic acid was refluxed under argon for 3 h, and then evaporated the solvent to dryness. 1HNMR (CDCl3) δ 8.63 (br s, 2H), 8.40 (d, 4H), 7.52 (d, 2H), 7.20 (m, 2H), 6.68 (br s, 2H), 6.57 (s, 2H), 6.09 (q, 2H), 5.82 (br s, 2H), 5.52 (s, 2H); MS (CI) 397(M+H)

b) Preparation of 5,10,15,20-tetra(3-pyridyl)-21-monothiaporphyrin (T14). To the solution of compound (T7) formed last step 0.07 mL (1.0 mmol) of pyrrole and 0.2 mL (2.0 mmol) of 3-pyridinecarboxaldehyde were added with stirring, the resulting mixture was refluxed under air for 2.5 h. After removal of solvent, the residue was separated with column and PTLC (chloroform-methanol 95:5) to give (T14) (17%). 1HNMR (CDCl3) δ 9.74 (s, 2H), 9.46 (s, 2H), 9.41 (s, 2H), 9.03 (br s, 4H), 8.93 (s, 2H), 8.68 (d, J=4.44 Hz, 2H), 8.61 (d, J=4.47 Hz, 2H), 8.57 (d, 2H), 8.51 (t, 2H), 7.82 (t, 2H), 7.77 (t, 2H), −2.76 (s, 1H); MS (CI) 636(M+H).

c) Preparation of DL98. Prepared from (T14) with iodomethane according to general method, yield 55%. 1HNMR (DMSO-d₆) δ 10.18 (s, 2H), 10.08 (m, 4H), 9.62 (t, 4H), 9.46 (s, 2H), 9.41 (d, 2H), 9.33 (d, 2H), 9.02 (d, J=4.40 Hz, 2H), 8.91 (d, J=4.52 Hz, 2H), 8.68 (t, 2H), 8.63 (t, 2H), 4.75 (s, 6H), 4.71 (s, 6H), −3.12 (s, 1H); HRMS (FAB) (M−H) calcd 694.2751, obsd 694.2742, C44H36N7S.

IX. Design and Synthesis of Selenaporphyrins

As was true with thiaporphyrins, methods of synthesizing of di- and mono-selenaporphyrins with cationic electron withdrawing groups were not previously known in the art. It is also expected that the selenaporphyrins of the present invention will not exhibit photo-induced skin toxicity. The absorption spectra of selenaporphyrins exhibit even a further red shift in the absorbtion spectra than thiaporphyrins. A study of the emission properties of the selenaporphyrins with fluorescence spectroscopy revealed that selenaporphyrins do not show any fluorescence. This is probably due to the substitution of the heavier selenium atoms at the porphyrin core. With these observations, selenaporphyrins are expected to show no skin toxicity.

The X-ray crystal structure (Latos-Grazynski et al., 1996) of selenaporphyrins reveals a more nonplanar structure than the thia porphyrins due to the steric hindrance of the heavier selenium atom with the atoms in the core of the porphyrin ring.

A. Synthesis of Various Selenaporphyrins

The selenium-containing porphyrins were synthesized by normal acid-catalyzed condensation using pyrrole or pyridine carboxaldehydes with diols obtained from selenophene. The cationic porphyrins were obtained from the freebase porphyrins by reaction with alkyl iodides in chloroform or a mixture of chloroform and nitromethane followed by ion exchange.

The intermediates selenophene diols were found to be insoluble in dichloromethane, and hence the Lindsey method was not suitable for the synthesis of cationic pyridyl porphyrins. Further, the duration of the oxidation reaction in these cases are generally higher, and consequently Ulman's original method was mainly applied in these synthetic procedures.

The selenaporphyrins may be coordinated to a metal. A range of metal complexes (transition metals and lanthamides) can be prepared to identify those metals that confer the best combination of low photosentization, strong telomerase inhibition, and low cytotoxicity on the porphyrin. The preferred metal ions may be used in the more elaborate porphyrin conjugates.

1) Synthesis of 3-pyridyl-diselenaporphyrin

The synthesis of 3-pyridyl-diselenaporphyrin involved condensation of pyrrole and 2,5-bis(α-hydroxy-α-pyridyl methyl)selenophene in propionic acid under air. The synthesis of the key precursor selenophene diol was acquired in two steps that involve lithiation followed by reaction with 3-pyridine carboxaldehyde. The synthesis is shown in Scheme IX.

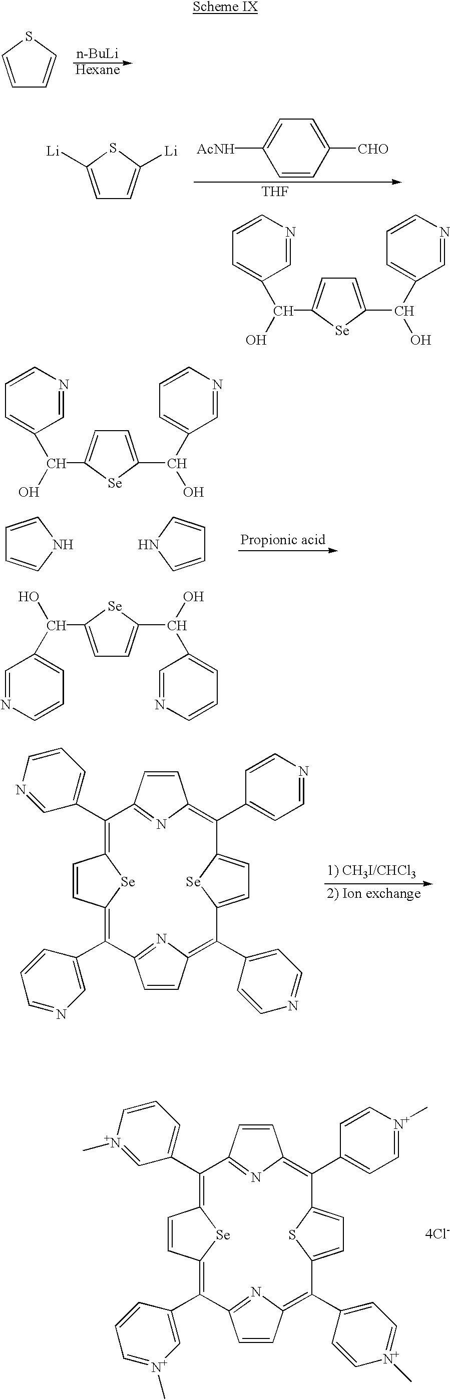

2) Synthesis of 3-pyridyl-monoselenaporphyrin

The intermediate 5,10-dipyridyl-16-selenatripyrrane was synthesized by the reaction of 2,5-bis(α-hydroxy-α-pyridyl methyl)selenophene with excess pyrrole in propionic acid. This intermediate was further reacted with pyrrole and 3-pyridine-carboxaldehyde in propionic acid under air to give 3-pyridyl-monoselenaporphyrin. The synthesis is shown in Scheme IX.

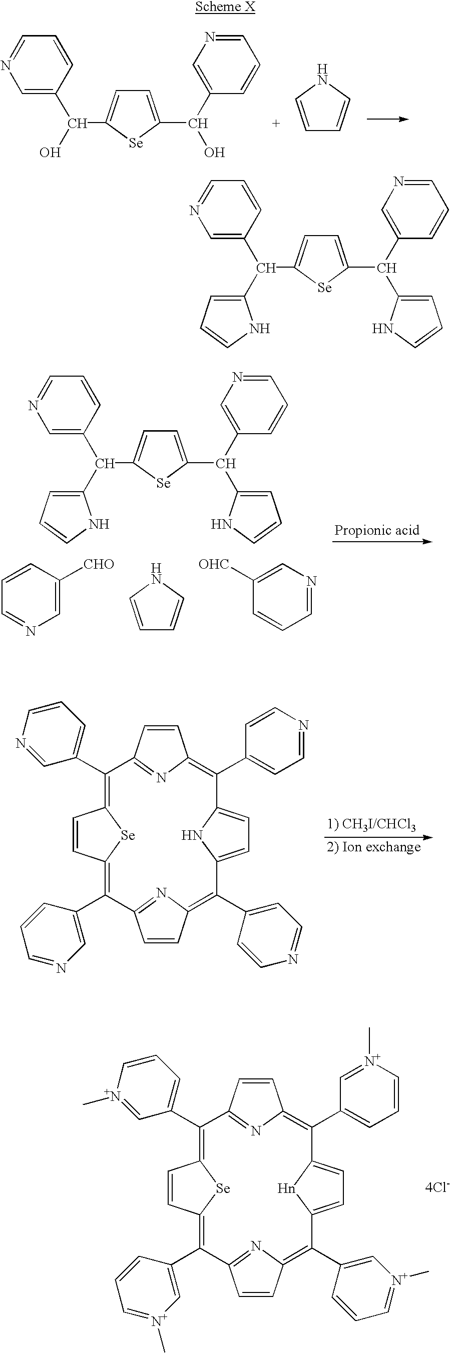

a) Preparation of 2,5-bis(3-pyridylhydroxymethyl)thiophene. To a three-necked, round-bottomed flask flushed with argon was added 150 mL of anhydrous hexane, 8.6 mL (0.057 mol) of TMEDA, and 23 mL (2.5 M in hexane) (0.057 mol) of n-butyllithium. 1.76 mL (0.019 mol) of selenophene was then added at room temperature, and the mixture was refluxed for 1 h. After cooling to room temperature, 5.4 mL of 3-pyridine carboxaldehyde (0.057 mol) in dry THF (30 mL) was added drop-wise to the ice cooled reaction mixture. After this addition was completed, the mixture was allowed to attain room temperature. 20 mL of methanol and NH4Cl were added separately with stirring. The organic layers were combined, washed with water, and dried over sodium sulfate. After removal of solvent, the residue was purified by chromatography on silica gel using chloroform-methanol (8:1) as an eluent.

b) Preparation of 5,10,15,20-tetra (3-pyridyl)-21,23-diselenaporphyrin. A mixture of 1.2 g (3.49 mmol) of compound and 0.363 mL (5.24 mmol) was dissolved in 500 mL of propionic acid. The mixture was heated to reflux for 7 h. After cooling to room temperature, the solvent was evaporated to dryness under high vacuum. The residue was purified on chromatography on basic alumina using chloroform-methanol as an eluent.

X. Design and Synthesis of Carotenoid Porphyrins

In addition to modifying the core of porphyrins in order to overcome the problem of photo-induced skin toxicity, another consideration of modification involves introducing a carotene moiety to one or more phenyl rings that are attached to the porphyrin core. It has been found that the covalent bonding of these carotenoid porphyrins efficiently quenches the photo activity of the porphyrin (Reddi et al., 1994). Because of the extended conjugations from the porphine ring to the carotenoid, this will quench the production of active oxygen species and therefore reduce the photoactivity of the porphyrin. The present invention discloses various novel cationic carotenoid porphyrins and other porphyrin analogs that do not exhibit photo-induced skin toxicity.

A. Synthesis of Carotenoid Porphyrins

The structures of the carotenoid porphyrins described herein are shown in table 2. Additionally, the structures of various starting materials referenced herein are shown in table 3. In table 3, each of these starting materials has number associated with the compound, such as “(1).” This is used as a shorthand designation of the compound. Preceeding references to that designation, such as “compound (1),” reference the compound in table 3 associated with that designation.

TABLE 2

| 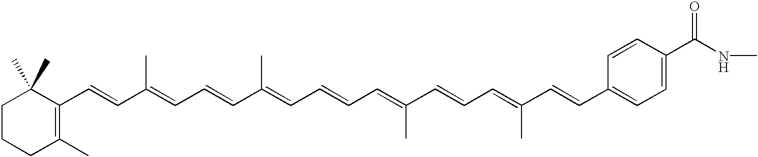 | C₈₂H₈₁Cl₃N₈O =DL99 |
|---|---|
| 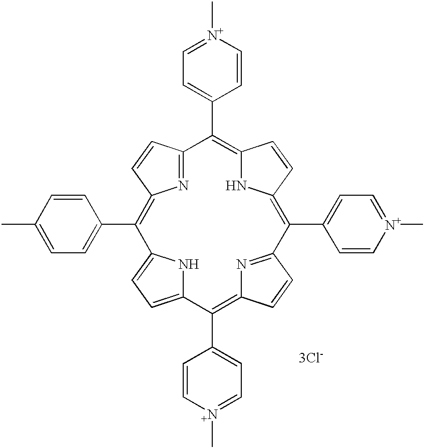 | |
| 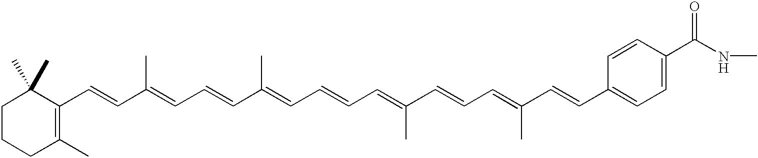 | C₈₂H₈₁Cl₃N₈O =DL100 |
| 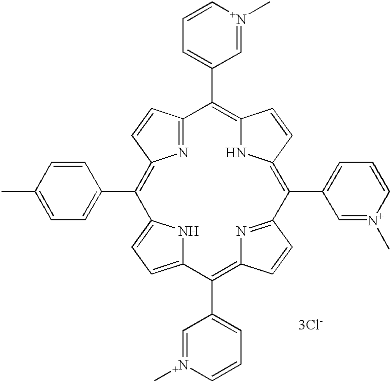 | |
| 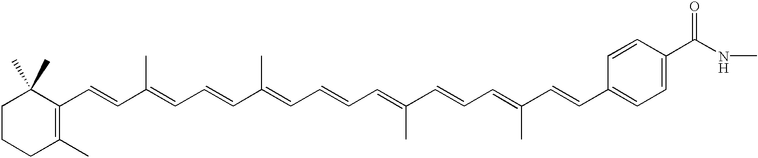 | C₉₄H₈₇Cl₃N₈O =1451.10 DL101 |
| 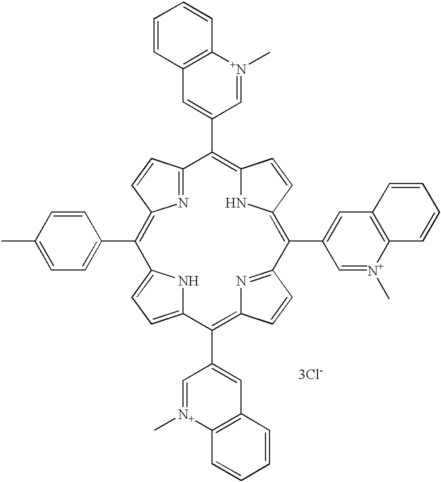 | |

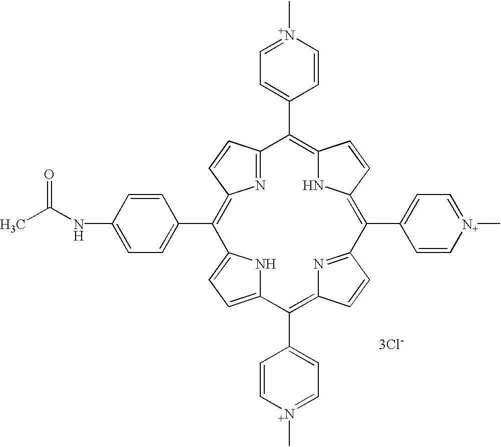

C₄₆H₃₉Cl₃N₈O = 826.22
DL102

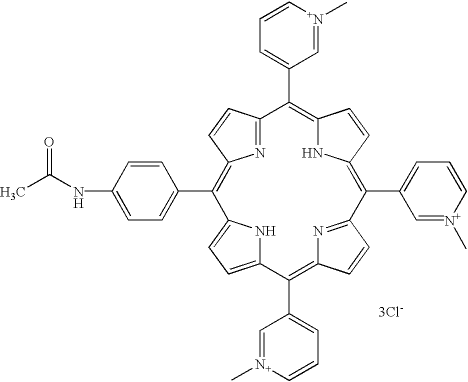

C₄₆H₃₉Cl₃N₈O = 826 22
DL103

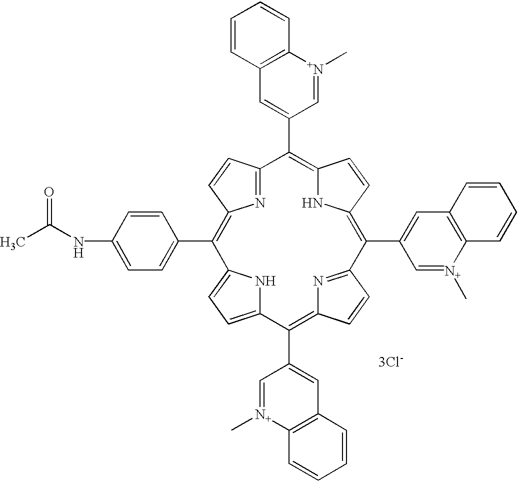

C₅₈H₄₅Cl₃N₈O = 976.389
DL104

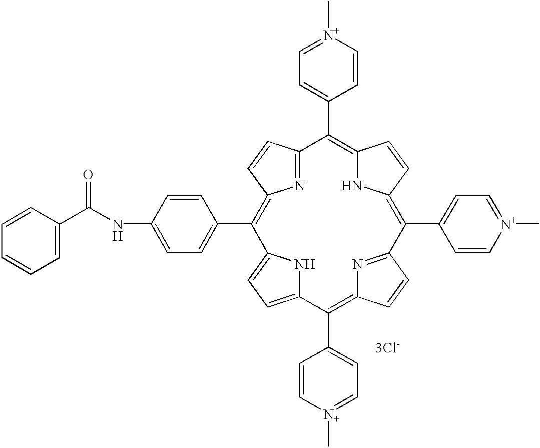

C₅₁H₄₁Cl₃N₈O = 888.279
DL105

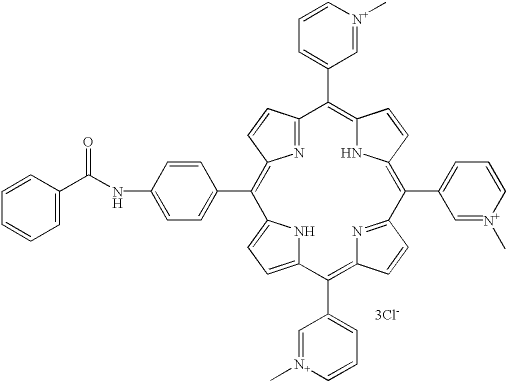

C₅₁H₄₁Cl₃N₈O = 888.279
DL106

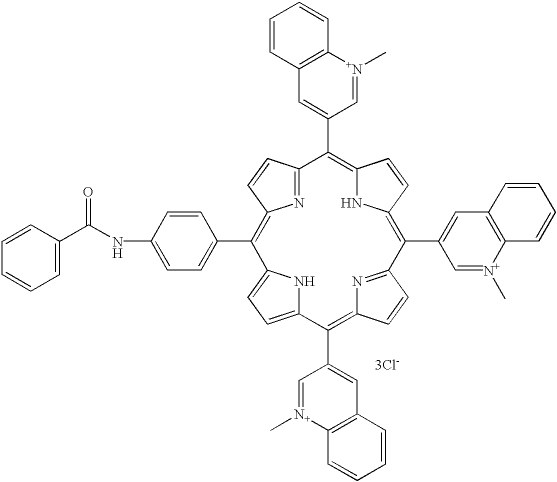

C₆₃H₄₇Cl₃N₈O = 1038.459
DL107

TABLE 3

| 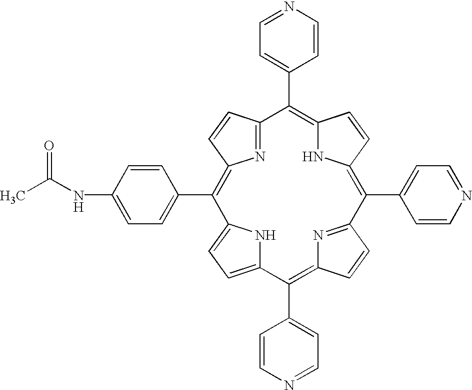 | (1) |
|---|---|
| 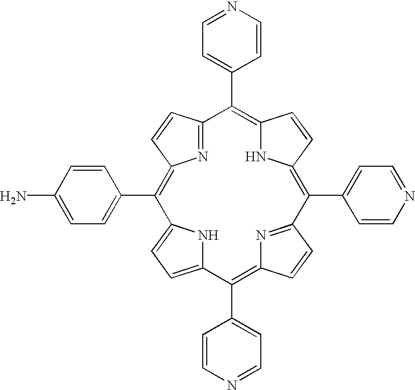 | (2) |
| 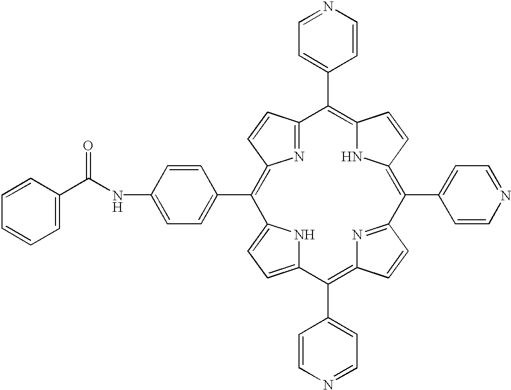 | (3) |
| 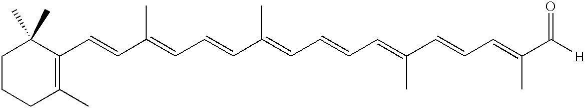 | (4) |
| 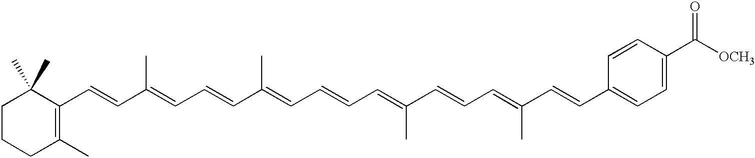 | (5) |
| 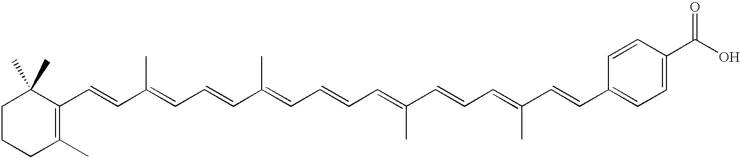 | (6) |
| 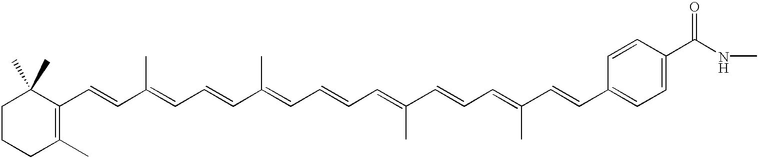 | (7) |
| 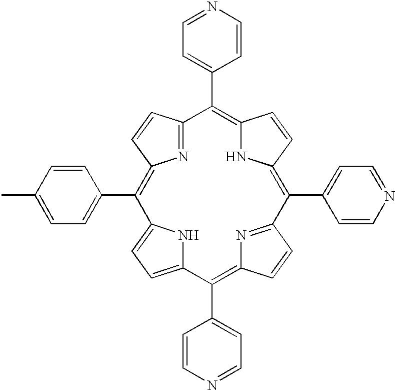 | |
| 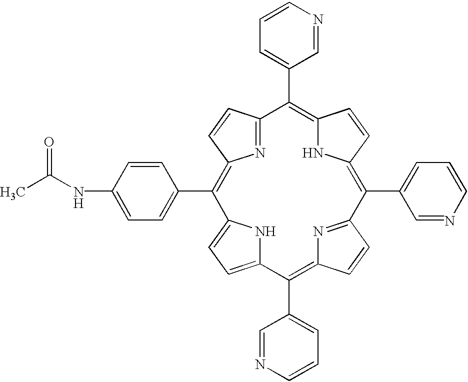 | (8) |
| 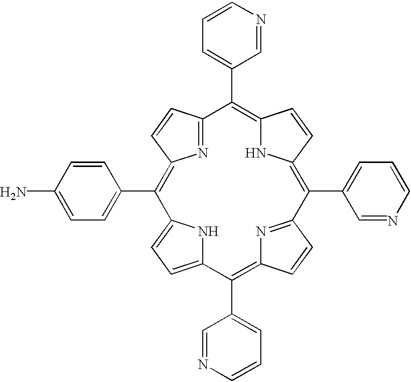 | (9) |
| 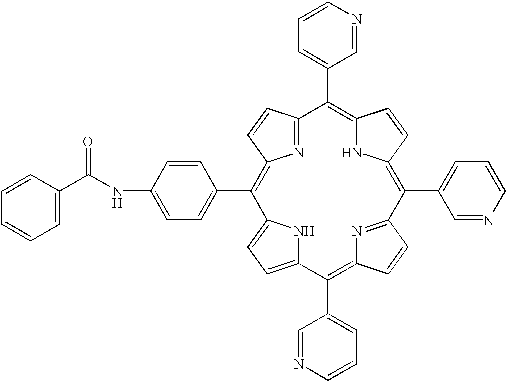 | (10) |
| 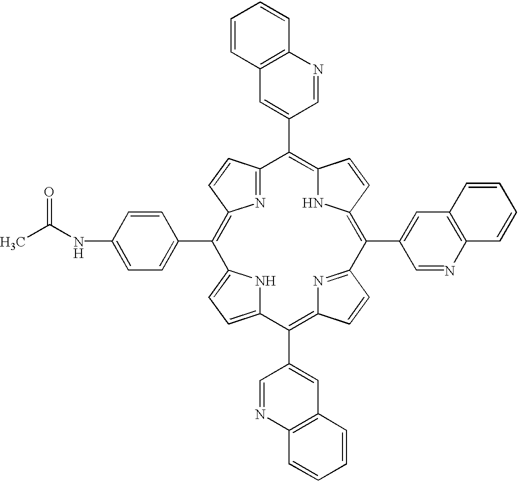 | (11) |
| 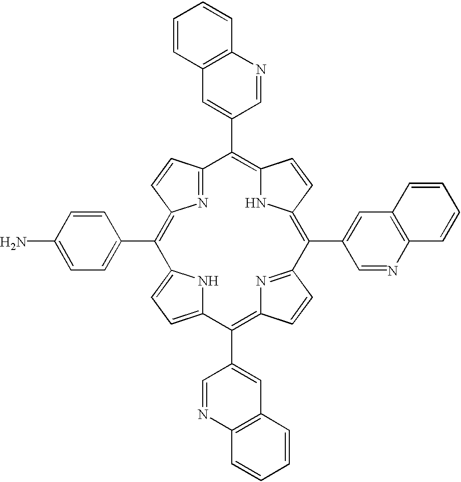 | (12) |
| 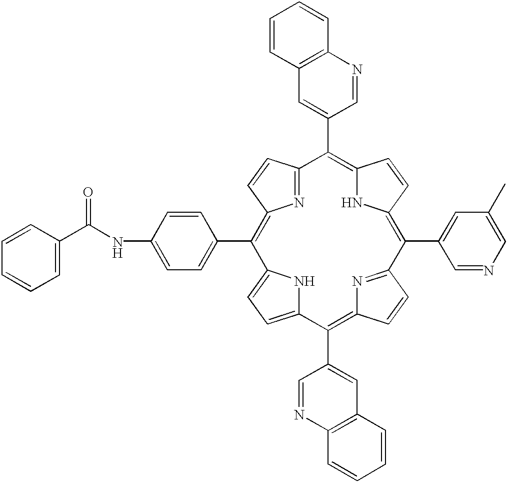 | (13) |
| 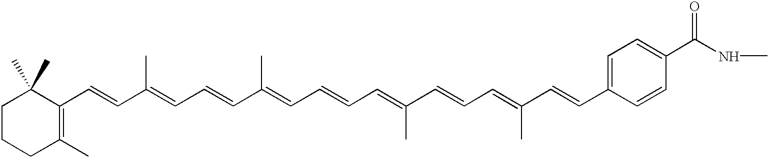 | (14) |
| 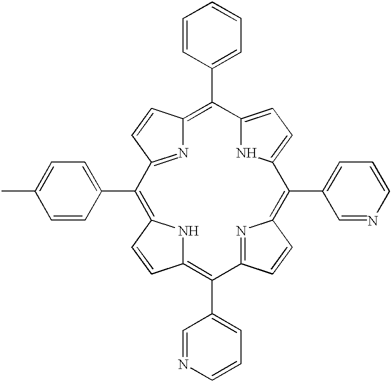 | |
| 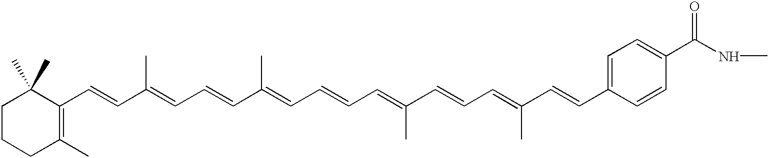 | (15) |
| 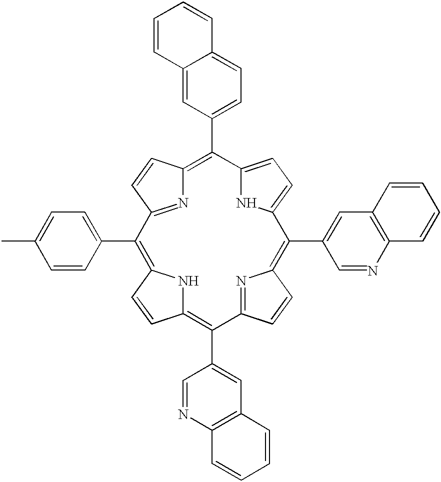 | |

1. Preparation of 5-(4-acetamidophenyl)-10,15,20-tri(N-methyl-3-pyridyl)-porphyrin Chloride (DL103)

a) Preparation of 5-(4-acetamidophenyl)-10,15,20-tri(3-pyridyl)-porphyrin (8). Prepared from condensation of 2.0 g (12.3 mmol) of 4-acetamidobenzaldehyde, 1.32 g (12.3 mmol) of 3-pyridinecarboxaldehyde, and 1.65 g (24.6 mmol) of pyrrole in propionic acid according to general method. Chromatography on silica gel using chloroform-methanol (95:5) as eluent gave (8) (5.2%). 1HNMR (CDCl3) δ 10.39 (s, 1H), 9.44 (s, 3H), 9.05 (d, 3H), 8.93-8.79 (m, 8H), 8.51 (br d, 3H), 8.15 (d, J=8.1 Hz, 2H), 7.77 (m, 5H), 2.30 (s, 3H), −2.85 (s, 2H); MS (FAB) 675(M+H).

b) Preparation of 5-(4-acetamidophenyl)-10,15,20-tri(N-methyl-3-pyridyl)-porphyrin chloride (DL103). Prepared from (8) with iodomethane according to general method, yield 73.2%. 1HNMR (DMSO-d₆) δ 10.75 (s, 1H), 10.07 (s, 3H), 9.59 (m, 3H), 9.34 (br m, 3H), 9.21-9.04 (m, 8H), 8.61 (br m, 3H), 8.16 (br s, 4H), 4.71 (s, 9H), 2.26 (s, 3H), −3.00 (s, 2H); HRMS (FAB) (M) calcd 719.3247, obsd 719.3213, C46H39N80.

c) Preparation of 5-(4-aminophenyl)-10,15,20-tri(3-pyridyl)-porphyrin (9). 170 mg (0.252 mmol) of compound (8) was dissolved in 20 mL of TFA and 20 mL of conc. HCl was added at room temperature. The resulting mixture was heated at 80-85° C. for 24 h, then cooled to 0° C. It was diluted with 10 mL of water, neutralized with 1N NaOH to pH 8-9, extracted with chloroform, and then the organic layer was died over Na2SO4. After removal of solvent, the residue was purified by chromatography on silica gel using chloroform-methanol (95:5) as eluent gave (9) (80.9%); 1HNMR (CDCl3) δ 9.44 (s, 3H), 9.05 (d, J=4.9 Hz, 3H), 9.01 (d, J=4.6 Hz, 2H), 8.82 (s, 4H), 8.78 (d, J=4.6 Hz, 2H), 8.52 (d, J=7.4 Hz, 3H), 7.97 (d, J=8.1 Hz, 2H), 7.75 (dd, J=4.9 Hz, J=7.4 Hz, 3H), 7.05 (d, J=8.1 Hz, 2H), −2.79 (s, 2H); MS (FAB) 633(M+H).

d) Preparation of 5-(4-benzoylamidophenyl)-10,15,20-tri(4-pyridyl)-porphyrin (10). To a solution of 30 mg (0.0474 mmol) of compound (9) in CHCl3 was added 0.3 mL of triethylamine, the resulting mixture was stirred for 10 min at room temperature, then 24.2 mg (0.172 mmol) of benzoyl chloride was added. The mixture was stirred for another 2 h. After removal of solvent, the residue was purified by PTLC (chloroform-methanol 8:1) giving (10) (94.4%). 1HNMR (CDCl₃) δ 9.42 (s, 3H), 9.03 (br s, 3H), 8.96 (d, J=3.9 Hz, 2H), 9.03 (s, 4H), 9.01 (d, J=3.9 Hz, 2H), 8.51 (br d, 3H), 8.25-8.03 (m, 6H), 7.75 (br t, 3H), 7.63-7.57 (m, 3H), −2.82 (s, 2H); MS (FAB) (M+H) calcd 737.2777, obsd 737.2805, C48H33N8O.

2. Preparation of 5-(4-benzoylamidophenyl)-10,15,20-tri(N-methyl-3-pyridyl)porphyrin Chloride (DL106)

a) Prepared from (10) with iodomethane according to general method, yield 71%. 1HNMR (DMSO-d₆) δ 10.95 (s, 1H), 10.13 (br s, 3H), 9.67 (br d, 3H), 9.44 (br d, 3H), 9.29-9.16 (m, 8H), 8.68 (m, 3H), 8.43 (br d, 2H), 8.31 (br d, 2H), 8.21 (br d, 2H), 7.72 (m, 3H), 4.75 (s, 9H), −2.84 (s, 2H); HRMS (FAB) (M) calcd 781.3403, obsd 781.3371, C51H41N8O.

3. 5-(4-acetamidophenyl)-10,15,20-tri(N-methyl-3-quinolyl)-porphyrin Chloride (DL 104)

a) Preparation of 5-(4-acetamidophenyl)-10,15,20-tri(3-quinolyl)-porphyrin (11). Prepared from condensation of 2.0 g (12.3 mmol) of 4-acetamidobenzaldehyde, 1.93 g (12.3 mmol) of 3-quinolinecarboxaldehyde, and 1.65 g (24.6 mmol) of pyrrole in propionic acid according to general method. Chromatography on silica gel using chloroform-methanol (95:5) as eluent gave (11) (5.2%). 1HNMR (CDCl3) δ 10.41 (s, 1H), 9.78 (s, 3H), 8.96 (s, 3H), 8.92 (d, J=4.8 Hz, 2H), 8.85 (s, 4H), 8.81 (d, J=4.8 Hz, 2H), 8.46 (d, 3H), 8.16-8.06 (m, 5H), 7.98-7.87 (m, 5H), 7.77 (t, 3H), 2.25 (s, 3H), −2.68 (s, 2H); MS (FAB) 825(M+H).

b) DL104 prepared from (11) with iodomethane according to general method, yield 81.5%. 1HNMR (DMSO-d6) δ 10.89 (s, 1H), 10.72 (br s, 3H), 10.13 (br s, 3H), 9.35-9.08 (m, 8H), 8.92 (d, 3H), 8.83 (m, 3H), 8.58 (t, 3H), 8.33 (t, 3H), 8.19 (br,s 4H), 4.95 (s, 9H), 2.26 (s, 3H), −2.87 (s, 2H); HRMS (FAB) (M) calcd 869.3716, obsd 869.3656, C58H45N8O.

4. Preparation of 5-(4-benzoylamidophenyl)-10,15,20-tri(N-methyl-3-quinolyl)porphyrin Chloride (DL 107)

a) Preparation of 5-(4-aminophenyl)-10,15,20-tri(3-quinolyl)-porphyrin (12). 343 mg (0.416 mmol) of compound (11) was dissolved in 30 mL of TFA and 30 mL of conc. HCl was added at room temperature. The resulting mixture was heated at 80-85° C. for 24 h, then cooled to 0° C. It was diluted with 15 mL of water, neutralized with 1N NaOH to pH 8-9, extracted with chloroform, and then the organic layer was died over Na2SO4. After removal of solvent, the residue was purified by chromatography on silica gel using chloroform-methanol (95:5) as eluent gave (12) (82%); 1HNMR (CDCl3) δ 9.83 (d, J=1.98 Hz, 3H), 9.06 (d, J=4.7 Hz, 2H), 9.00 (s, 3H), 8.89 (s, 4H), 8.87 (d, J=4.7 Hz, 2H), 8.52 (d, 3H), 8.14 (d, 3H), 8.04-7.98 (m, 5H), 7.84 (t, 3H), 7.11 (d, J=8.3 Hz, 2H), −2.61 (s, 2H); HRMS (FAB) (M+H) calcd 783.2946, obsd 783.2985, C53H35N8.

b) Preparation of 5-(4-benzoylamidophenyl)-10,15,20-tri(4-quinolyl)-porphyrin (13). To a solution of 35 mg (0.0447 mmol) of compound (12) in CHCl3 was added 0.3 mL of triethylamine, the resulting mixture was stirred for 10 min at room temperature, then 24.2 mg (0.172 mmol) of benzoyl chloride was added. The mixture was stirred for another 2 h. After removal of solvent, the residue was purified by PTLC (chloroform-methanol 8:1) giving (13) (88.1%). 1HNMR (CDCl3) δ 9.78 (s, 3H), 8.98 (br s, 5H), 8.87 (br s, 6H), 8.46 (br d, 3H), 8.38 (s, 1H, NH), 8.23 (br s, 2H), 8.10 (m, 5H), 8.02 (d, 2H), 7.98 (t, 3H), 7.80 (t, 3H), 7.63-7.54 (m, 3H), −2.64 (s, 2H); MS (FAB) (M+H) calcd 887.3215, obsd 887.3247, C60H39N8O.

c) Preparation of DL107. Prepared from (13) with iodomethane according to general method, yield 80.4%. 1HNMR (DMSO-d6) δ 10.68 (br s, 3H), 10.11 (br s, 3H), 9.33-9.10 (m, 8H), 8.92 (br d, 3H), 8.80 (m, 6H), 8.56-8.12 (m, 10H), 7.68 (m, 3H), 4.95 (s, 9H), −2.85 (s, 2H); HRMS (FAB) (M) calcd 931.3867, obsd 931.3813, C63H47N8O.

5. Preparation of 5-{4-[4-(7′-apo-7′-β-carotenyl)benzoylamino]phenyl}-10,15,20-tri(N-methyl-3-pyridyl)porphyrin Chloride (DL 100)

a) Preparation of 5-{4-[4-(7′-apo-7′-β-carotenyl)benzoyl-amido]phenyl}-10,15,20-tri(3-pyridyl)porphyrin (14). Prepared from condensation of compound (6) with compound (9) according to the general method (14). Yield 20.3%. 1HNMR (CDCl3) δ 9.45 (s, 3H), 9.04 (d, 3H), 8.97 (d, 2H), 8.84 (br s, 4H), 8.81 (d, 2H), 8.50 (br d, 5H), 8.27 (s, H), 8.22 (d, 2H), 7.98 (d, J=7.94 Hz, 2H), 7.73 (m, 3H), 7.60 (d, J=7.91 Hz, 2H), 7.05 (d, 1H), 6.71-6.60 (m, 5H), 6.48-6.12 (m, 8H), 2.16-1.03 (m, 27H), −2.91 (s, 2H); HRMS (CI) (M+H) calcd 1149.5907, obsd 1149.5928, C79H73N8O.

b) Preparation of DL100. Prepared from (14) with iodomethane according to general method, yield 83%. 1HNMR (DMSO-d₆) 10.03 (br s, 3H), 9.56 (br s, 3H), 9.33-9.13 (m, 8H), 8.60 (br s, 3H), 8.36-8.12 (m, 9H), 7.76 (d, 2H), 7.73 (d, 2H), 7.25-6.16 (m, 13H), 4.68 (br s, 9H), 2.34-0.82 (m, 27H), −3.03 (s, 2H); HRMS (FAB) (M) calcd 1193.6533, obsd 1193.6558, C82H81N8O.

6. Preparation of 5-{4-[4-(7′-apo-7′-β-carotenyl)benzoylamino]phenyl}-10,15,20-tri(N-methyl-3-quinolyl)porphyrin chloride (DL 101)

a) Preparation of 5-{4-[4-(7′-apo-7′-β-carotenyl)benzoyl-amido]phenyl}-10,15,20-tri(3-quinolyl)porphyrin (15). Prepared from condensation of compound (6) with compound (12) according to the general method to give (15). Yield 31.1%. 1HNMR (CDCl3) δ 9.81 (br s, 3H), 9.02-8.91 (br d, 11H), 8.46 (d, 3H), 8.33 (s, 1H), 8.27 (m, 2H), 8.18-8.08 (m, 5H), 8.01 (m, 5H), 7.83 (t, 3H), 7.58 (d, 2H), 7.05 (d, 1H), 6.72-6.61 (m, 5H), 6.47-6.13 (m, 8H), 2.15-1.04 (m, 27H), −2.68 (s, 2H); HRMS (CI) (M+H) calcd 1299.6377, obsd 1299.6340, C91H78N8O.

b) Preparation of DL101. Prepared from (15) with iodomethane according to general method, yield 74%. 1HNMR (DMSO-d₆) 10.64 (br s, 3H), 10.09 (br s, 3H), 9.31-9.10 (m, 8H), 8.90 (d, 3H), 8.77 (m, 4H), 8.55 (t, 3H), 8.37-8.13 (m, 9H), 7.74 (d, 2H), 7.31-6.15 (m, 13H), 4.95 (br s, 9H), 2.35-0.84 (m, 27H), −2.84 (s, 2H); HRMS (CI) (M+H) calcd 1343.7003, obsd 1343.7049, C94H87N8O.

7. Preparation of 5-(4-acetamidophenyl)-10,15,20-tri(N-methyl-4-pyridyl)porphyrin Chloride (DL102)

a) Preparation of 5-(4-acetamidophenyl)-10,15,20-tri(4-pyridyl)-porphyrin (1). Prepared from condensation of 2.0 g (12.3 mmol) of 4-acetamidobenzaldehyde, 1.32 g (12.3 mmol) of 4-pyridinecarboxaldehyde, and 2.46 g (24.6 mmol) of pyrrole in propionic acid according to general method. Chromatography on silica gel using chloroform-methanol (95:5) as eluent gave (1) (7%). 1HN (DMSO-d₆) δ 10.41 (s, 1H), 9.01 (d, J=5.6 Hz, 6H), 8.94-8.84 (m, 8H), 8.23 (dd, J=5.6 Hz, 6H), 8.12 (d, J=8.4 Hz, 2H), 8.03 (d, J=8.4 Hz, 2H), 2.24 (s, 3H), −3.02 (s, 2H); MS (CI) 675(M+H).

b) Preparation of DL102. Prepared from (1) with iodomethane according to general method, yield 80%. 1HNMR (DMSO-d₆) δ 10.68 (s, 1H), 9.52 (br d, 6H), 9.23-8.97 (m, 14H), 8.24 (br d, 4H), 4.72 (br s, 9H), −3.01 (s, 2H); HRMS (FAB) (M) calcd 719.3247, obsd 719.3237, C46H39N8O.

8. Preparation of 5-(4-benzoylamidophenyl)-10,15,20-tri(N-methyl-4-pyridyl)porphyrin Chloride (DL105)

a) Preparation of 5-(4-aminophenyl)-10,15,20-tri(4-pyridyl)-porphyrin (2). 55.7 mg (0.083 mmol) of compound (1) was dissolved in 7 mL of TFA and 7.5 mL of conc. HCl was added at room temperature. The resulting mixture was heated at 80-85° C. for 24 h, then cooled to 0° C. It was diluted with 20mL of water, neutralized with 1N NaOH to pH 8-9, extracted with chloroform, and then the organic layer was died over Na2SO4. After removal of solvent, the residue was purified by chromatography on silica gel using chloroform-methanol (95:5) as eluent gave (2) (80.6%); 1HNMR (CDCl3) δ 9.00 (d, J=5.7 Hz, 6H), 8.98-8.77 (m, 8H), 8.12 (d, J=5.7 Hz, 6H), 7.95 (d, J=8.1 Hz, 2H), 7.05 (d, J=8.1 Hz, 2H), −2.89 (s, 2H); MS (CI) 633(M+H).

b) Preparation of 5-(4-benzoylamidophenyl)-10,15,20-tri(4-pyridyl)-porphyrin (3). To a solution of 30 mg (0.0474 mmol) of compound (2) in CHCl3 was added 0.3 mL of triethylamine, the resulting mixture was stirred for 10 min at room temperature, then 24.2 mg (0.172 mmol) of benzoyl chloride was added. The mixture was stirred for another 2 h. After removal of solvent, the residue was purified by PTLC (chloroform-methanol 8:1) giving (3) (86.4%). 1HNMR (CDCl3) δ 9.00-8.80 (m, 8H), 8.98 (d, J=5.7 Hz, 6H), 8.22-8.18 (m, 2H), 8.15 (d, J=5.7 Hz, 6H), 8.11 (d, 2H), 8.07 (d, 2H), 7.63-7.57 (m, 3H), −2.98 (s, 2H); MS (FAB) 737(M+H).

c) Preparation of DL105. Prepared from (3) with iodomethane according to general method, yield 80%. 1HNMR (DMSO-d₆) δ 11.02 (s, H), 9.62 (br s, 6H), 9.24-9.12 (m, 14H), 8.50 (br s, 2H), 8.22 (br d, 2H), 8.17 (br s, 2H), 7.75 (m, 3H), 4.89 (br s, 9H), −2.88 (s, 2H); HRMS (FAB) (M) calcd 781.3403, obsd 781.33092, C51H41N8O.

9. Preparation of 5-{4-[4-(7′-apo-7′-β-carotenyl)benzoyl-amido]phenyl}-10,15,20-tri(N-methyl-4-pyridyl)porphyrin chloride (DL99)

The desired carotenoic acid was synthesized from 8′-apo-β-carotenal by a Wittig reaction with 4-carbomethoxybenzyltriphenylphosphonium bromide using sodium methoxide as the base, followed by basic hydrolysis. The coupling of the chromophores through the amide linkage was accomplished by following the acid chloride of the carotenoic acid, by treatment of the acid with thionyl chloride, and the immediate reaction of it with the appropriate amino-substituted tetraarylporphyrin (Gust et al., 1992). DL99 was prepared according to Scheme XII. A detailed description of the steps is provided below.

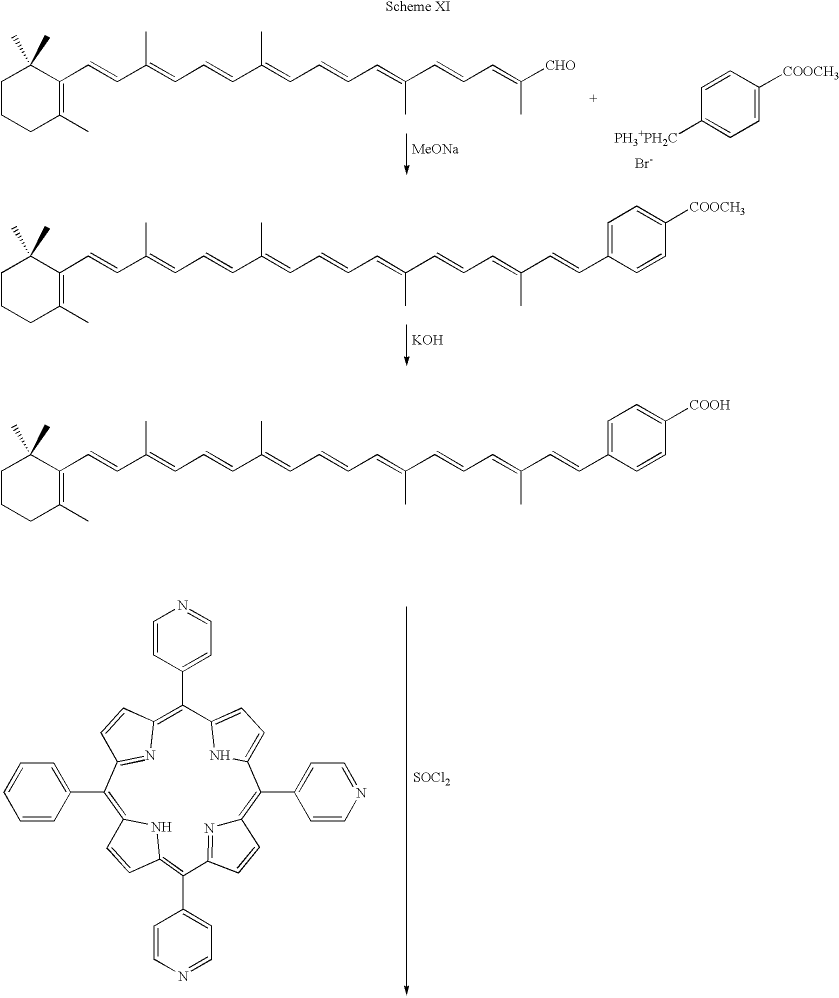

a) Preparation of 4-carbomethixybenzyltriphenylphosphonium bromide (4). The mixture of 1.5 g (6.55 mmol) of methyl-a-bromo-p-toluate, 1.72 g (6.55 mmol) of triphenylphosphine in 50 mL of toluene was refluxed for 2 h under argon, and then cooled to room temperature. After filtration, the residue was washed with dry toluene. The white solid was dried under vacuum to give (4) (87%).

b) Preparation of 7′-apo-7′-(4-carbomethoxyphenyl)-β-carotene (5). To a solution of 5.0 g (2.4 mmol) of 8′-apo-β-carotenal (20%) in DMSO was added 1.4 g (2.9 mmol) of compound (4), and 0.17 g (3.1 mmol) of sodium methoxide. The suspension is heated to 80° C. and stirred under argon. After 16 h a supplemental amount of both the phosphonium bromide (1.18 g, 2.4 mmol) and sodium methoxide (0.13 g, 2.4 mmol) was added and the reaction mixture was stirred for an additional 16 h. The resulting mixture was then poured into ethyl ether and the organic layer was washed with water to remove all traces of DMSO. The ether layer was dried over MgSO4 and evaporated, the residue was recrystallized from dichloromethane-methanol giving (5) (43.4%). UV λmax (toluene) (nm) 458, 482, 514; 1HNMR (CDCl3) δ 7.94 (d, J=8.4 Hz, 2H), 7.44 (d, J=8.4 Hz, 2H), 7.00-6.12 (m, 14H), 3.88 (s, 3H), 2.02-1.15 (m, 21H), 1.00 (s, 6H); HRMS (CI) (M+H) calcd 549.3733, obsd 549.3693, C39H49O2.

c) Preparation of 7′-apo-7′-(4-carboxyphenyl)-β-carotene (6). 115 mg (0.21 mmol) of compound (5) was dissolved in 16mL of a mixture of THF and methanol (3:1). To this solution was added 2 mL of 10% aqueous KOH, and the mixture was stirred under argon for 48 h at room temperature. The reaction mixture was adjusted with 1N HCl to pH 1-2, and then extracted with CHCl3. The organic layer was dried over Na2SO4, and the solvent was evaporated to yield (6) (82%). 1HNMR (CDCl3) δ 8.00 (d, J=8.4 Hz, 2H), 7.46 (d, J=8.4 Hz, 2H), 7.10-6.12 (m, 14H), 2.02-1.19 (m, 21H), 1.00 (s, 6H); HRMS (CI) (M+H) calcd 535.3576, obsd 535.3527, C38H47O2.

d) Preparation of 5-{4-[4-(7′-apo-7′-β-carotenyl)benzoyl-amido]phenyl}-10,15,20-tri(4-pyridyl)porphyrin (7). To a 50 mL flask were added 70 mg (0.13 mmol) of compound (6), 20 mL of dry benzene, 30 μL (0.4 mmol) of thionyl chloride, and 80 μL of dry pyridine (dry over KOH). The initial orange suspension was rapidly converted into dark red color. After stirring the solution for 45 min under argon at room temperature, the solvent was distilled under vacuum. 20nL of anhydrous benzene was added and evaporated to dryness under vacuum to remove the excess thionyl chloride. The residue that remained was dissolved in 30 mL of dry chloroform and added to a solution of 82.25 mg (0.13 mmol) of compound (2), which was dissolved in 60 mL dry CHCl3 and 0.2 mL of dry pyridine. The resulting mixture was stirred under argon overnight, then evaporated to dryness. The residue was purified by chromatography on silica gel with chloroform-methanol (10:1) to give (7) (46.2%). 1HNMR (CDCl3) δ 8.99 (d, 2H), 8.93 (br d, 6H), 8.82 (m, 6H), 8.43 (s, H), 8.20-8.09 (m, 4H), 8.06 (br d, 6H), 7.98 (d, J=8.1 Hz, 2H), 7.56 (d, J=8.1 Hz, 2H), 7.05-6.13 (m, 14H), 2.14-1.20 (m, 21H), 1.01 (s, 6H), −2.91 (s, 2H); HRMS (CI) (M+H) calcd 1149.5907, obsd 1149.5917, C79H73N8O.

e) Preparation of DL99. Prepared from (7) with iodomethane according to general method, yield 83%. 1HNMR (DMSO-d₆) δ 9.49 (br s, 6H), 9.13-8.99 (m, 10H), 8.34-8.10 (m, 6H), 7.75-7.60 (m, 6H), 7.32-6.15 (m, 14H), 4.71 (br s, 9H), 2.14-1.20 (m, 21H), 1.01 (s, 6H), −2.99 (s, 2H); HRMS (FAB) (M) calcd 1193.6533, obsd 1193.6578, C82H81N8O.

XI. Biological Data

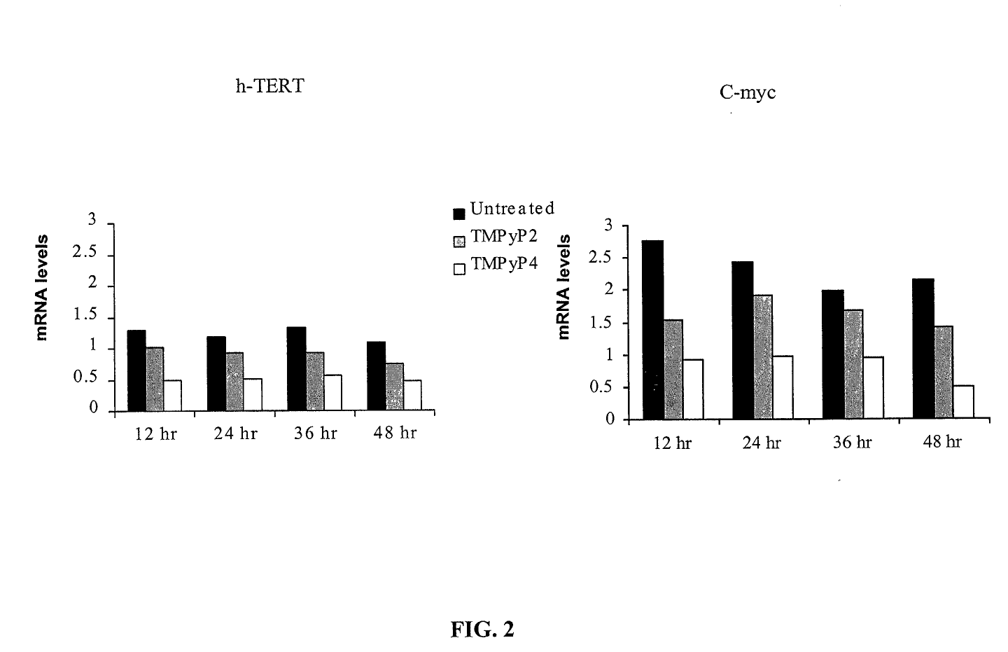

In addition to inhibition of telomerase and production of anaphase bridges in sea urchin embryos, telomerase enzyme levels are also depressed. Since c-myc controls levels of hTERT, the catalytic subunit of telomerase, the effect of TMPyP4 on c-myc and hTERT expression in HeLa cells was also evaluated. The results of a time-course experiment are shown in FIG. 2. TMPyP4, but not TNPyP2, down-regulates c-myc and hTERT mRNA levels in a time-dependent manner. TMPyP2 is a positional isomer of TMPyP4 that does not appreciably interact with G-quadruplexes. These results are consistent with the idea that TMPyP4 down-regulates telomerase through stabilization of the G-quadruplex structure in the P1 promoter of c-myc. Since the G-quadruplex is the inactive form of the P1 promoter, this down-regulates c-myc and downstream genes, including hTERT. The differential effect of TMPyP2 and TMPyP4 on gene expression using a c-DNA chip array is shown in Tables 4 and 5.

TABLE 4
Genes Affected by Both TMPyP4 and TMPyP2 Treatment

| INDUCED GENES | DOWN-REGULATED GENES |
|---|---|
| Oxidation Reduction Genes | Maeallothionein Genes |
| Cystathionase | Maeallothionein IIH |
| Lactate dehydrogenase | Maeallothionein 1L |
| Cytochrome p450 | Maeallothionein 1H |
| Thioredoxin | Maeallothionein 2A |
| Superoxide dismutase-1 | |
| Superoxide dismutase-2 | |
| Glutathione S-transferase M4 | |
| Proteasome Genes | |
| Macropain alpha | |
| Macropain 26S | |

TABLE 5
Effects of TMPyP4 Treatment on Gene Expression

| INDUCED GENES | DOWN REGULATED GENES |
|---|---|
| Apoptosis Genes | c-Myc-Associated Genes |
| Caspase 1 | c-Myc |
| Cell-Signaling Genes | Ornithine decarboxylase |
| TGF-β | CDC 25A |
| CD47 | Cell-Cycle Genes |
| CD9 | CDK-4 |
| CO-29 | CDK-6 |
| RAB-1A | Cyclin B1 |
| RAB9 | Cell Signaling and Oncogenes |
| Proliferation-associated gene A | c-Fos |
| DNA Repair Genes | c-Myc |
| MLH1 | Jun-B |
| ERCC5 | c-Myb |
| Heat Shock Genes | STAT-1 |
| Hsp 27 | |
| Hsp 10 | |

TMPyP4 has a preferential effect on telomere shortening in multiple myeloma cells and this results in cell crisis and apoptosis. These effects are much less in TMPyP2-treated cells.

### REFERENCES

The following references, to the extent that they provide exemplary procedural or other details supplementary to those set forth herein, are specifically incorporated herein by reference.

Allshire, Gosden, Cross, Cranston, Rout, Sugawara, Szostak, Fantes, Hastie, “Telomeric repeat from T. thermophilia cross hybridizes with human telomeres,” Nature, 332:656-659, 1988.

Anantha, Azam, Sheardy, “Porphyrin binding to quadrupled T4G4,” Biochemistry, 37(9):2709-2714, 1998.

Arthanari, Basu, Kawano, Bolton, “Fluorescent dyes specific for quadruplex DNA,” Nucleic Acid Res., 26(16):3724-3728, 1998.

Berberich and Postel, “PuF/NM23-H2/NDPK-B transactivates a human c-myc promoter-CAT gene via a functional nuclease hypersensitive element,” Oncogene, 10:2343-2347, 1995.

Blackburn, “Structure and function of telomeres,” Nature, 350:569-573, 1991.

Blackburn, Greider, Eds., “In: Telomeres,” Cold Spring Harbor Press, New York, 1995.

Burger, Double, Newell, “Inhibition of telomerase activity by cisplatin in human testicular cancer cells, ” Eur. J. Cancer, 33:638-644, 1997.

Chadwick, Wilbe. J. Chem. Soc., Perkin Trans. I, 99, 887, 1977.

Counter, Avillion, Le Feuvre, Stewart, Greider, Harley, Baccetti, EMBO J., 11: 1921-922, 1992.

Davis, Firulli, Kinniburgh, Proc. Natl. Acad. Sci. U.S.A., 86:9682-9686, 1989.

Fedoroff, Yu; Rangan, Chemeris, Hurley, “Cationic porphyrins promote the formation of I-motif DNA and bind peripherally by a nonintercalative mechanism,” Biochemistry, 39:15083-15090, 2000.

Fletcher, Salazar, Chen, “Human Telomerase Inhibition by 7-Deaza-2′-deoxypurine Nucleoside Triphosphates,” Biochemistry., 35:15611-15617, 1996.

Gehring, Leroy, Gueron, Nature, 363:561-565, 1993.

Gust, Moore, Moore, Liddell, Methods in Enzymology, 213:87, 1992.

Han and Hurley, Trends Pharm. Sci., 21:136-142, 2000.

Han, Rangan, Fedoroff, Yu, Hurley, J. Am. Chem. Soc., in revision, 2000.

Haq, I., Ladbury, J. E., B. Z. and Jenkins, T. C. (1996). Molecular Anchoring of Duplex and Triplex DNA by Disubstituted Anthracene-9,10-diones: Calorimetric, UV Melting and Competition Dialysis Studies. J. Am. Chem. Soc., 118, 10693-10701.

Haq, Trent, Chowdhry, Jenkins, J. Am. Chem. Soc., 121:1768-1779, 1999.

Harley, Futcher, Greider, “Telomeres shorten during aging of human fibroblasts,” Nature, 345:458460, 1990.

Harley, Kim, Prowse, Weinrich, Hirsch, West, Bacchetti, Hirte, Counter, Greider, Wright, Shay, “Telomerase, Cell Immortality, and Cancer,” Cold Spring Harbor Symp., Quant. Biol., 59:307-315, 1994.

Kerwin, Curr. Pharm. Des., 6:441-478, 2000.

Kim, Piatyszek, Prowse, Harley, West, Ho, Coviello, Wright, Weinrich, Shay, Science, 266:2011-2015, 1994.

Latos-Grazynski, Lisowski, Szterenberg, Olmstead, Balch, J. Org. Chem., 56:4043, 1991.

Latos-Grazynski, Pacholska, Chmielewski, Olmstead, Balch, Angew. Chem. Int. Ed. Engl., 34:2252, 1995.

Latos-Grazynski, Pacholska, Chmielewski, Olmstead, Balch, Inorg. Chem., 35:566, 1996.

Lee and Kim, Tetrahedron Lett., 3935, 1997.

Leroy, Gehring, Kettani, Gueron, Biochemistry, 32:6019 6031, 1993.

Leroy, Gueron, Mergny, Hélène, Nucleic Acids Res., 22: 1600-1606), 1994.

Li and Czuchajowski, Tetrahedron Lett., 1629, 1994.

Lindsey and Wagner, J. Org. Chem., 54:828, 1989.

Lingner, Hughes, Shevchenko, Mann, Lundblad, Cech, Science, 276:561-567, 1997.

Lipscomb, Zou, Presnell, Woo, Peek, Plaskon, Williams, Biochemistry, 35:2818-2823, 1996.

Marcinokowska, Ziolkowski, Pacholska, Latos-Grazynski, Chmielewski, Radzikowski, Anticancer Res., 17:3313, 1997.

Mata, Joshi, Palen, Pirruccello, Jackson, Elias, Paige, Medlin, Iveson, Toxicol. Appl. Pharmacol., 144:189-197, 1997.

Michelotti, Michelotti, Pullner, Duncan, Eick, Levens, Mol. Cell. Biol, 16:2656-2669, 1996.

Morin, J. Natl. Cancer Inst., 87:859-861, 1995.

Neidle and Kelland, Anti-Cancer Drug Design, 13:341-347, 2000.

Norton, Piatyszek, Wright, Shay, Corey, Nature Biotech., 14:615-619, 1996.

Parkinson, Brit. J. Cancer, 73:1-4, 1996.

Perche, Saint-Ruf, Buu-Hoi, Chem. Soc., Perkin I., 260, 1972.

Postel, Flint, Kessler, Hogan, Proc. Natl. Acad. Sci. U.S.A., 88:8227-8231, 1991.

Raymond, Sun, Chen, Windie, Von Hoff, Curr. Opinion Biotech., 7:583-591, 1996.

Reddi, Segalla, Jori, Kerrigan, Liddell, Moore, Moore, Gust, Br. J. Cancer, 69:40, 1994.

Salazar, Thompson, Kerwin, Hurley, Biochemistry, 35:16110-16115, 1996.

Shippen-Lentz, Blackburn, “Functional Evidence for a RNA Template in Telomerase,” Science, 247, 546-552, 1990.

Siebenlist, Henninghausen, Battey, Leder, Cell, 37:381-391, 1984.

Simonson, Pecinka, Kubista, Nucleic Acids Res., 26:1167-1172, 1998.

Srinivasan, Sridevi, Chandrashekar, Tetrahedron Lett., 4149, 1997.

Strahl and Blackburn, Mol. Cell Biol., 16:53-65, 1996.

Ulman and Manassen, Am. Chem. Soc., 97:6540, 1975.

Ulman and Manassen, Chem. Soc., Perkin Trans. 1,101, 7055, 1979.

Ulman, Manassen, Frolow, Rabinowich, Inorg. Chem., 1981, 20, 1987.

Ulman, Manassen, Frolow, Rabinowich, J. Am. Chem. Soc., 101:7055, 1979.

Ulman, Manassen, Frolow, Rabinowich, Tetrahedron Lett., 167, 1978.

Ulman, Manassen, Frolow, Rabinowich, Tetrahedron Lett., 1885, 1978.

Wahren, Tetrahedron, 2773, 1964.

Wang and Patel, Structure, 1:263-282, 1993.

Weitzmann, Woodford, Usdin, J. Biol. Chem., 271:20958-20964, 1996.

Wheelhouse, Sun, Han, Han, Hurley, J. Am. Chem. Soc., 120:3261, 1998.

Yale, J. Am. Chem. Soc., 70:254, 1948.

Zahler, Williamson, Cech, Prescott, “Inhibittion of Telomerase by G-Tetrad DNA Structures,” Nature, 350:718-720, 1991.

Ziolkowski, Milach, Symonowicz, Chmielewski, Latos-Grazynski, Marcinkowska, Tumori, 81:364, 1995.

What is claimed is:

1. A method of inhibiting the expression of c-myc in a cell comprising contacting the cell with a thiaporphyrin or a selenaporphyrin wherein the thiaporphyrin or the selenaporphyrin has a formula: 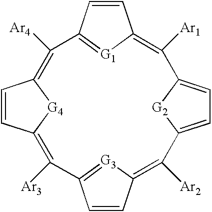
where one of G₁, G₂, G₃, or G₄ is S or Se, and the remainder are N,N, and NH; or
where two of G₁, G₂, G₃, or G₄ are either both S or both Se, two are N, and the two N are located opposite each other;
where Ar₁, Ar₂, Ar₃ and Ar₄ are H or independently 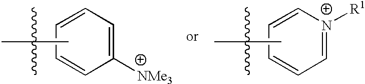
where R¹ is H, lower alkyl, —CH₂CH₂OH, CH₂OAc, or —CH₂CH₂CH₂SO₃⁻, 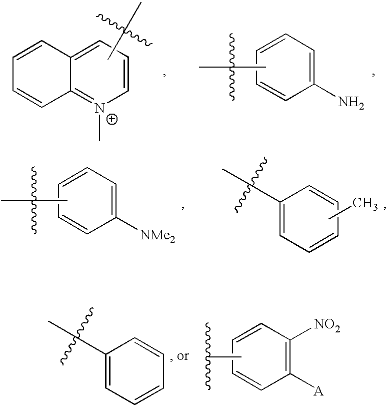
where A is H, OH, OMe, Cl or Me, or 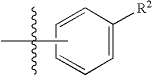
where R² is —CO₂H, CONH₂, CONHCH₂CH₂Br or NHCOCH₃;
or where Ar₁, Ar₂, Ar₃ and Ar₄ are independently 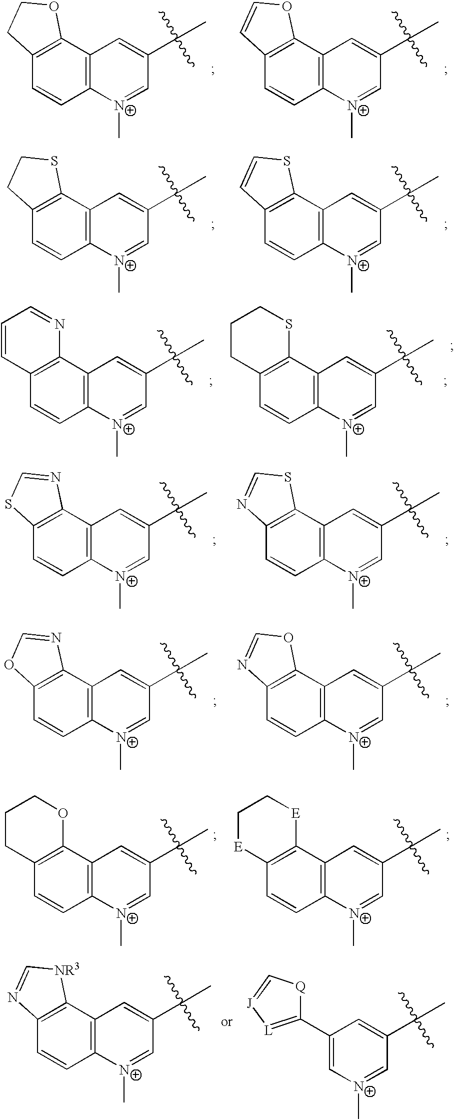
where Q is O, S, NH or NMe; J is CN or N; L is N or CH
where R³ is lower alkyl, and each E is independently CH₂, NH, NMe, O or S;
or where Ar₁, Ar₂, Ar₃ and Ar₄ are independently 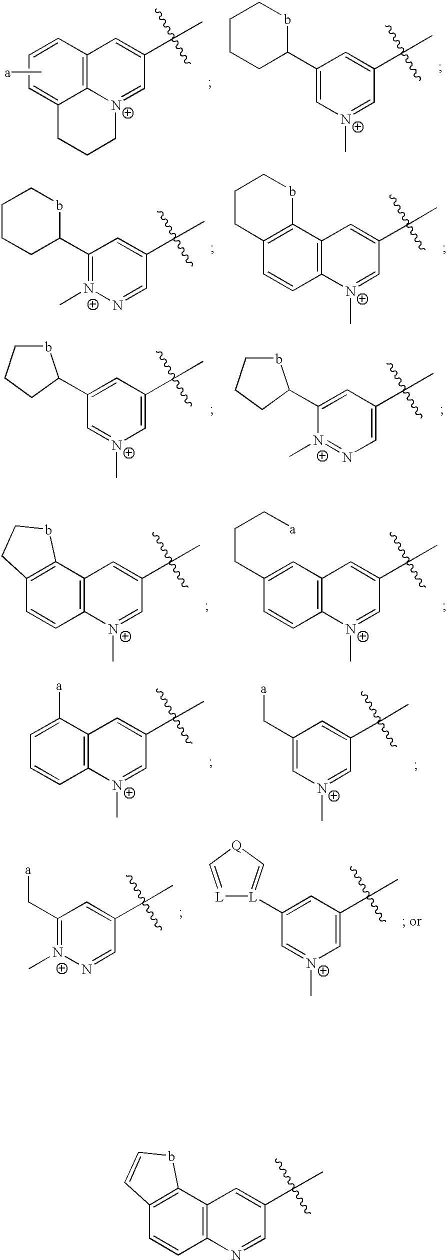
where a is NH₂, NHMe, NMe₂, OH, OMe, Sme; b is NH, NMe, SMe, O or S; Q is O, S, NH or NMe; each L is independently N or CH
or where Ar₁, Ar₂, Ar₃ and Ar₄ are independently 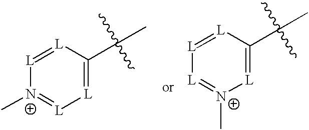
where each L is independently N or CH;
or where Ar₁, Ar₂, Ar₃ and Ar₄ are independently 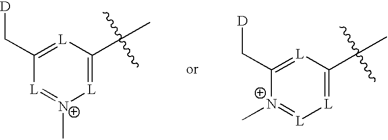
where each L is independently N or CH and D is NH₂, NHMe, NMe₂, OH, SH, SMe or CF₃;
or where Ar₁, Ar₂, Ar₃ and Ar₄ are independently 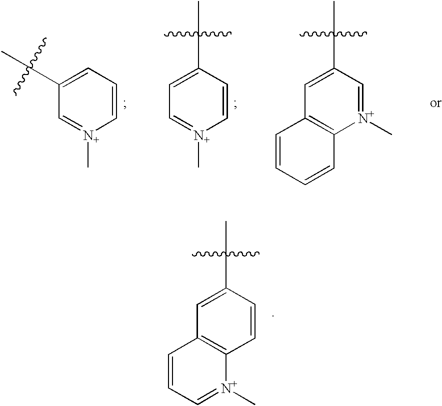
or where at least one, but not more than two, of Ar₁, Ar₂, Ar₃, or Ar₄ is 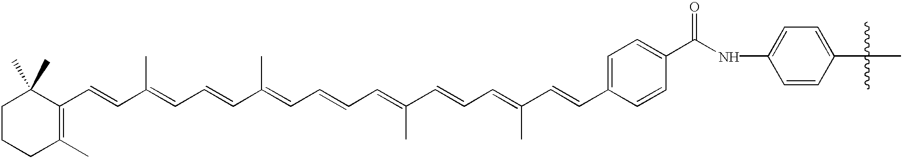
and the remainder of Ar₁, Ar₂, Ar₃, or Ar₄ are positively charged moieties;
or where at least one, but not more than two, of Ar₁, Ar₂, Ar₃, or Ar₄ is 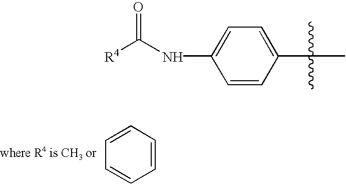
and the remainder of Ar₁, Ar₂, Ar₃, or Ar₄ are positively charged moieties.

2. The method of claim 1, where one of G₁, G₂, G₃, or G₄ is S or Se, and the remainder are N,N, and NH.

3. The method of claim 1, where two of G₁, G₂, G₃, or G₄ are either both S or both Se, two are N, and the two N are located opposite each other.

4. The method of claim 1, further comprising a metal coordinated to the thiaporphyrin or the selenaporphyrin, such that the thiaporphyrin or the selenaporphyrin has a formula: 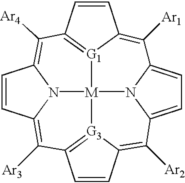
where G₁ is S or Se and G₃ is N; or
where G₁ and G₃ are both S or both Se;
where M is a metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

5. A method of inhibiting the expression of c-myc in a cell, comprising contacting the cell with a thiaporphyrin or a selenaporphyrin wherein the thiaporphyrin or the selenaporphyrin has a formula: 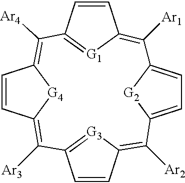
where one of G₁, G₂, G₃, or G₄ is S or Se, and the remainder are N,N, and NH; or
where two of G₁, G₂, G₃, or G₄ are either both S or both Se, two are N, and the two N are located opposite each other;
where Ar₁, Ar₂, Ar₃ and Ar₄ are independently 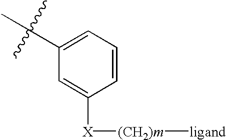
where m is 0-3, X is O, NH, CO, or CH₂, and where ligand is:

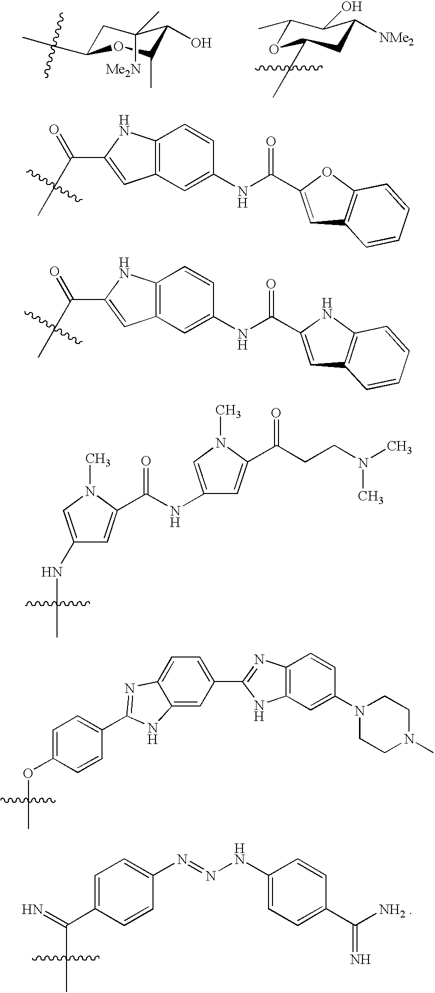

6. The method of claim 5, where one of G₁, G₂, G₃, or G₄ is S or Se, and the remainder are N,N, and NH.

7. The method of claim 5, where two of G₁, G₂, G₃, or G₄ are either both S or both Se, two are N, and the two N are located opposite each other.

8. The method of claim 5, further comprising a metal coordinated to the thiaporphyrin or the selenaporphyrin, such that the thiaporphyrin or the selenaporphyrin has a formula: 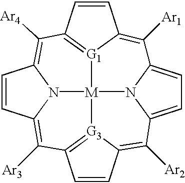
where G₁ is S or Se and G₃ is N; or
where G₁ and G₃ are both S or both Se;
where M is a metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

9. A method for cleaving telomeric DNA, comprising contacting the telomeric DNA with a thiaporphyrin or a selenaporphyrin having a formula: 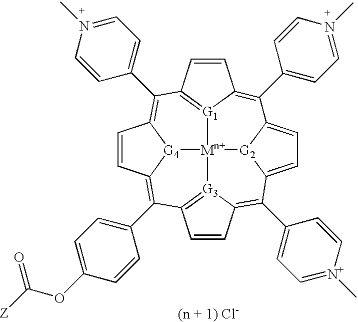
where one of G₁, G₂, G₃, or G₄ is S or Se, and the remainder are N; or
where two of G₁, G₂, G₃, or G₄ are either both S or both Se, two are N, and the two N are located opposite each other;
and where Z is Fe.EDTA, n is 1-3, and M is H⁺ or metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

10. The method of claim 9, where one of G₁, G₂, G₃, or G₄ is S or Se, and the remainder are N.

11. The method of claim 9, where two of G₁, G₂, G₃, or G₄ are either both S or both Se, two are N, and the two N are located opposite each other.

12. A method for covalently modifying telomeric DNA, comprising contacting telomeric DNA with a thiaporphyrin or a selenaporphyrin having a formula: 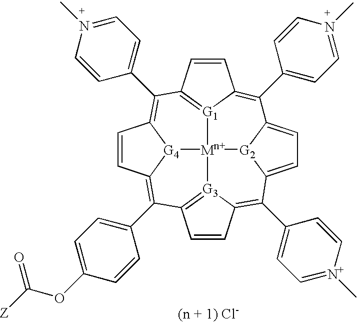
where one of G₁, G₂, G₃, or G₄ is S or Se, and the remainder are N; or
where two of G₁, G₂, G₃, or G₄ are either both S or both Se, two are N, and the two N are located opposite each other; 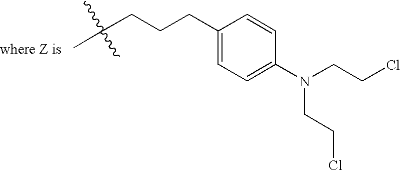
n is the charge on the metal M, and M is H⁺ or a metal cation selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

13. The method of claim 12, where one of G₁, G₂, G₃, or G₄ is S or Se, and the remainder are N.

14. The method of claim 12, where two of G₁, G₂, G₃, or G₄ are either both S or both Se, two are N, and the two N are located opposite each other.

15. The method of any of claims 12, 13, or 14 wherein the telomeric DNA is a G-quadruplex.

16. The method of any of claims 12, 13, or 14 wherein the telomeric DNA is human telomeromic DNA.

17. A compound having the formula: 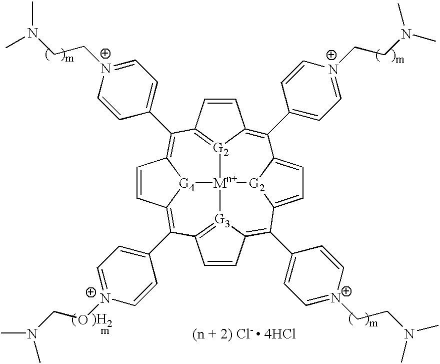
where one of G₁, G₂, G₃, or G₄ is S or Se, and the remainder are N; or
where two of G₁, G₂, G₃, or G₄ are either both S or both Se, two are N, and the two N are located opposite each other;
and where m is 0-3, n is the charge on the metal ion M, and M is H⁺ or a metal cation selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

18. The compound of claim 17, where one of G₁, G₂, G₃, or G₄ is S or Se, and the remainder are N.

19. The compound of claim 17, where two of G₁, G₂, G₃, or G₄ are either both S or both Se, two are N, and the two N are located opposite each other.

20. A compound having the formula: 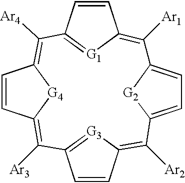
where one of G₁, G₂, G₃, or G₄ is S or Se, and the remainder are N,N, and NH; or
where two of G₁, G₂, G₃, or G₄ are either both S or both Se, two are N, and the two N are located opposite each other;
where Ar₁, Ar₂, Ar₃ and Ar₄ are H or independently 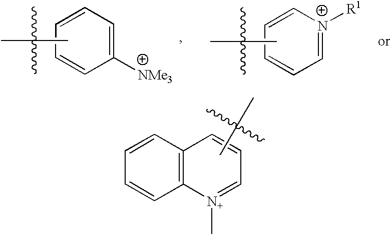
where R¹ is H, CH₃, lower alkyl, —CH₂CH₂OH, CH₂OAc, or —CH₂CH₂CH₂SO₃⁻, 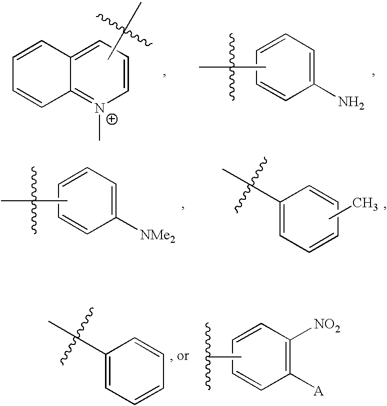
where A is H, OH, OMe, Cl or Me, 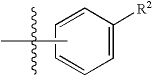
where R² is —CO₂H, CONH₂, CONHCH₂CH₂Br or NHCOCH₃ and salts thereof;
or where Ar₁, Ar₂, Ar₃ and Ar₄ are independently 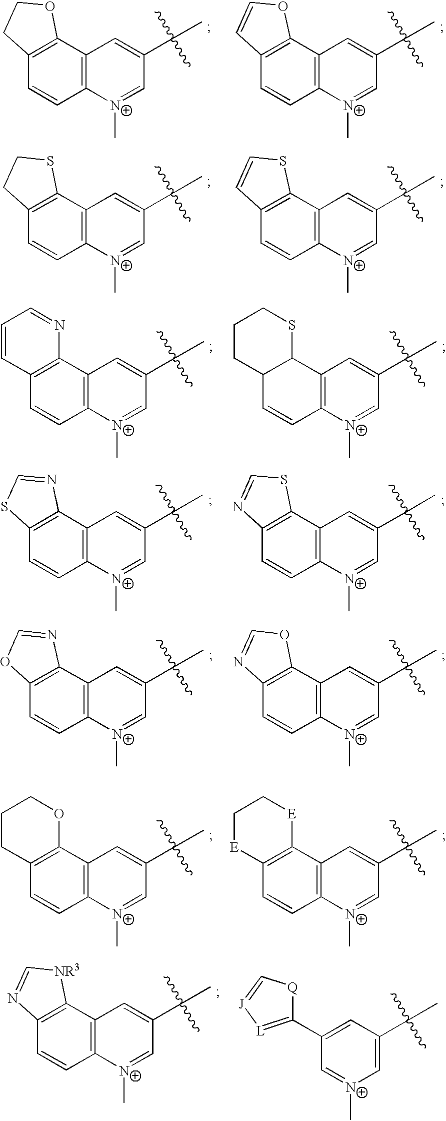
where Q is O, S, NH or NMe; J is CN or N; L is N or CH
where R³ is lower alkyl, and each E is independently CH₂, NH, NMe, O or S;
or where Ar₁, Ar₂, Ar₃ and Ar₄ are independently 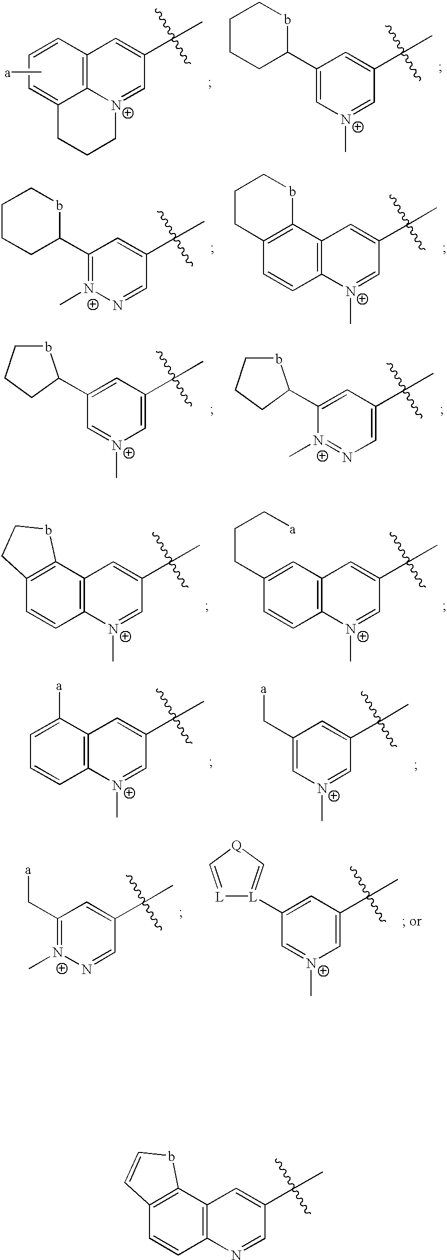
where a is NH₂, NHMe, NMe₂, OH, OMe, Sme; b is NH, NMe, SMe, O or S; is O, S, NH or NMe; each L is independently N or CH
or where Ar₁, Ar₂, Ar₃ and Ar₄ are independently 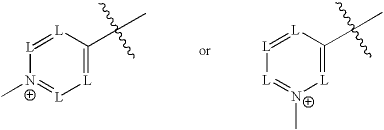
where each L is independently N or CH;
or where Ar₁, Ar₂, Ar₃ and Ar₄ are independently 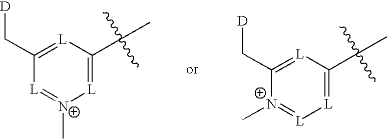
where each L is independently N or CH and D is NH₂, NHMe, NMe₂, OH, SH, SMe or CF₃;
or where Ar₁, Ar₂, Ar₃ and Ar₄ are independently 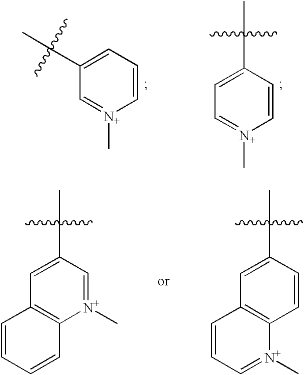
or where at least one, but not more than two, of Ar₁, Ar₂, Ar₃, or Ar₄ is 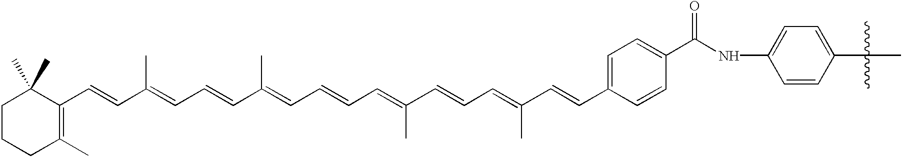
and the remainder of Ar₁, Ar₂, Ar₃, or Ar₄ are positively charged moieties;
or where at least one, but not more than two, of Ar₁, Ar₂, Ar₃, or Ar₄ is 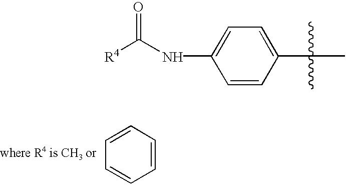
and the remainder of Ar₁, Ar₂, Ar₃, or Ar₄ are positively charged moieties.

21. The compound of claim 20, where one of G₁, G₂, G₃, or G₄ is S or Se, and the remainder are N,N, and NH.

22. The compound of claim 21, wherein the compound is 5,10-bis(N-methyl-6-quinolyl)-15,20-bis(N-methyl-4-pyridyl)-21-monothiaporphyrin chloride; 5,10-bis(N-methyl-3-pyridyl)-15,20-bis(N-methyl-4-pyridyl)-21-monothiaporphyrin chloride; or 5,10,15,20-tetra(N-methyl-3-pyridyl)-21-monothiaporphyrin chloride.

23. The compound of claim 20, where two of G₁, G₂, G₃, or G₄ are either both S or both Se, two are N, and the two N are located opposite each other.

24. The compound of claim 23, wherein the compound is 5,10,15,20-tetra(N-methyl-6-quinolyl)-21,23-dithiaporphyrin chloride; 5,10,15,20-tetra(N-methyl-3-pyridyl)-21,23-dithiaporphyrin chloride; or 5,10,15,20-tetra(N-methyl-3-quinolyl)-21,23-dithiaporphyrin chloride.

25. The compound of claim 20, further comprising a metal coordinated to the thiaporphyrin or the selenaporphyrin, such that the thiaporphyrin or the selenaporphyrin has a formula: 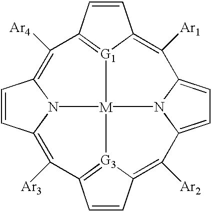
where G₁ is S or Se and G₃ is N; or
where G₁ and G₃ are both S or both Se;
where M is a metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu and salts thereof.

26. A pharmaceutical composition comprising the compound of claim 20 or 25.

27. A method of inhibiting cell proliferation comprising contacting said cell with an effective amount of a thiaporphyrin or a selenaporphyrin.

28. The method of claim 27 wherein the cell is a cancer cell.

29. The method of claim 28 wherein the cancer cell is a prostate or lymphoma cell.

30. The method of claim 28 wherein the cancer cell is a breast cancer cell.

31. The method of claim 30 wherein the breast cancer cell is a BT20, MCF-7m, 11S578t, HS576Bst or Hela cell.

32. The method of claim 27 wherein the cell is in a mammal.

33. A compound having the formula: 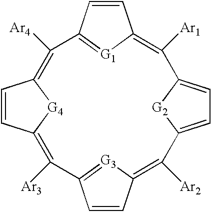
where one of G₁, G₂, G₃, or G₄ is S or Se, and the remainder are N,N, and NH; or
where two of G₁, G₂, G₃, or G₄ are either both S or both Se, two are N, and the two N are located opposite each other;
in which

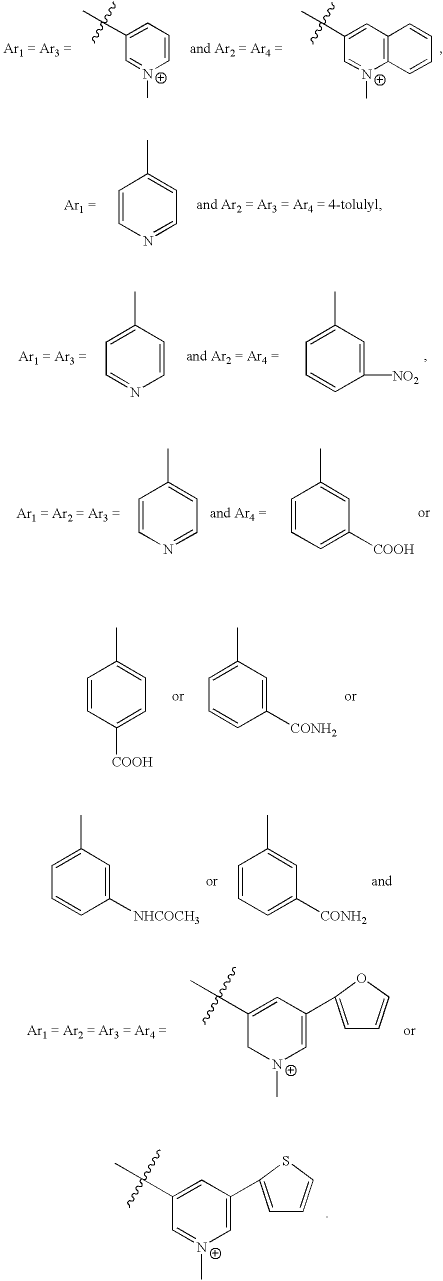

34. The compound of claim 33, where one of G₁, G₂, G₃, or G₄ is S or Se, and the remainder are N,N, and NH.

35. The compound of claim 33, where two of G₁, G₂, G₃, or G₄ are either both S or both Se, two are N, and the two N are located opposite each other.

36. A compound having the formula: 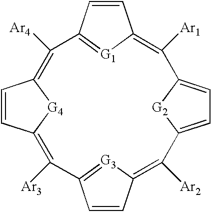
where one of G₁, G₂, G₃, or G₄ is S or Se, and the remainder are N,N, and NH; or
where two of G₁, G_{2, G}₃, or G₄ are either both S or both Se, two are N, and the two N are located opposite each other;

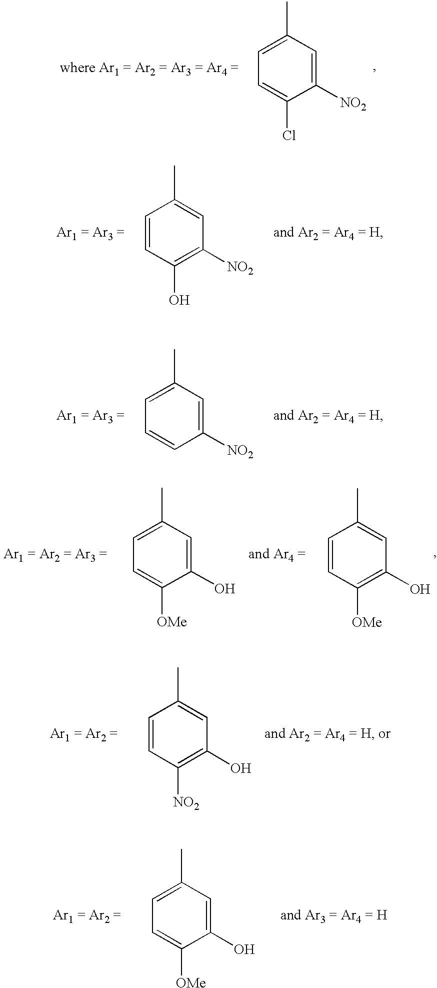

37. The compound of claim 36, where one of G₁, G₂, G₃, or G₄ is S or Se, and the remainder are N,N, and NH.

38. The compound of claim 36, where two of G₁, G₂, G₃, or G₄ are either both S or both Se, two are N, and the two N are located opposite each other.

39. A thiaporphyrin or a selenaporphyrin with the following formula: 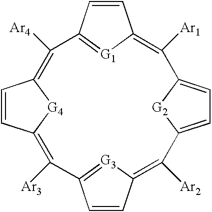
where one of G₁, G₂, G₃, or G₄ is S or Se, and the remainder are N,N, and NH; or where two of G₁, G₂, G₃, or G₄ are either both S or both Se, two are N, and the two N are located opposite each other;
and where Ar₁, Ar₂, Ar₃, and Ar₄ are positively charged moieties, and assume a nonplanar disposition with regard to said thiaporphyrin structure or said selenaporphyrin structure.

40. The compound of claim 39, where one of G₁, G₂, G₃, or G₄ is S or Se, and the remainder are N,N, and NH.

41. The compound of claim 39, where two of G₁, G₂, G₃, or G₄ are either both S or both Se, two are N, and the two N are located opposite each other.

42. The compound of claim 39, further comprising a metal coordinated to the thiaporphyrin or the selenaporphyrin such that the thiaporphyrin or the selenaporphyrin has a formula: 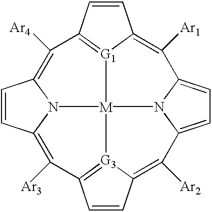
where G₁ is S or Se and G₃ is N; or
where G₁ and G₃ are both S or both Se;
where M is a metal.

43. A method of inhibiting the expression of c-myc in a cell, comprising contacting the cell with a thiaporphyrin or a selenaporphyrin wherein the thiaporphyrin or the selenaporphyrin has a formula: 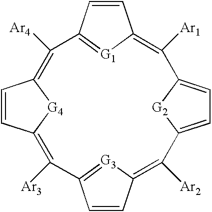
where one of G₁, G₂, G₃, or G₄ is S or Se, and the remainder are N,N, and NH; or
where two of G₁, G₂, G₃, or G₄ are either both S or both Se, two are N, and the two N are located opposite each other;
and where Ar₁, Ar₂, Ar₃, and Ar₄ are positively charged moieties, and assume a nonplanar disposition with regard to said thiaporphyrin structure or said selenaporphyrin structure.

44. The method of claim 43, where one of G₁, G₂, G₃, or G₄ is S or Se, and the remainder are N,N, and NH.

45. The method of claim 43, where two of G₁, G₂, G₃, or G₄ are either both S or both Se, two are N, and the two N are located opposite each other.

46. The method of claim 43, further comprising a metal coordinated to the thiaporphyrin or the selenaporphyrin such that the thiaporphyrin or the selenaporphyrin has a formula: 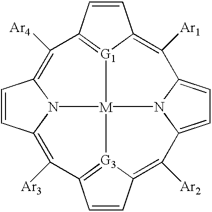
where G₁ is S or Se and G₃ is N; or
where G₁ and G₃ are both S or both Se;
where M is a metal.

47. A method of inhibiting proliferation of a cell comprising contacting said cell with a thiaporphyrin or a selenaporphyrin having the formula: 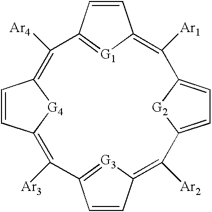
where one of G₁, G₂, G₃, or G₄ is S or Se, and the remainder are N,N, and NH; or
where two of G₁, G₂, G₃, or G₄ are either both S or both Se, two are N, and the two N are located opposite each other;
and where Ar₁, Ar₂, Ar₃, and Ar₄ are positively charged moieties, and assume a nonplanar disposition with regard to said thiaporphyrin structure or said selenoporhyrin structure.

48. The method of claim 47, where one of G₁, G₂, G₃, or G₄ is S or Se, and the remainder are N,N, and NH.

49. The method of claim 47, where two of G₁, G₂, G₃, or G₄ are either both S or both Se, two are N, and the two N are located opposite each other.

50. The method of claim 47, further comprising a metal coordinated to the thiaporphyrin or the selenaporphyrin such that the thiaporphyrin or the selenaporphyrin has a formula: 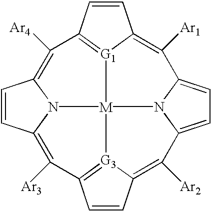
where G₁ is S or Se and G₃ is N; or
where G₁ and G₃ are both S or both Se;
where M is a metal.

51. A compound having the formula: 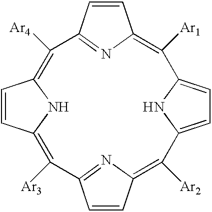
wherein at least one, but not more than two, of Ar₁, Ar₂, Ar₃, or Ar₄ is 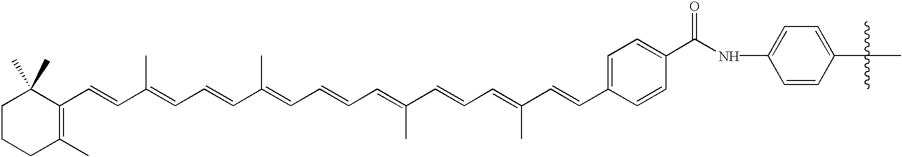
or where at least one, but not more than two, of Ar₁, Ar₂, Ar₃, or Ar₄ is 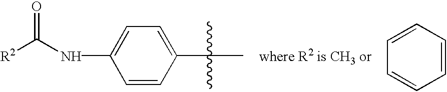
and the remainder of Ar₁, Ar₂, Ar₃, or Ar₄ are positively charged moieties.

52. The compound of claim 51, wherein the compound is 5-{4-[4-(7′-apo-7′-β-carotenyl)benzoylamido]phenyl}-10,15,20-tri(N-methyl-4-pyridyl)porphyrin chloride; 5-{4-[4-(7′-apo-7′-β-carotenyl)benzoylamido]phenyl}-10,15,20-tri(N-methyl-3-pyridyl)porphyrin chloride; 5-{4-[4-(7′-apo-7′-β-carotenyl)benzoylamino]phenyl}-10,15,20-tri(N-methyl-3-quinolyl)porphyrin chloride; 5-(4-acetamidophenyl)-10,15,20-tri(N-methyl-4-pyridyl)porphyrin chloride; 5-(4-acetamidophenyl)-10,15,20-tri(N-methyl-3-pyridyl)porphyrin chloride; 5-(4-acetamidophenyl)-10,15,20-tri(N-methyl-3-quinolyl)porphyrin chloride; 5-(4-benzoylamidophenyl)-10,15,20-tri(N-methyl-4-pyridyl)-porphyrin chloride; 5-(4-benzoylamidophenyl)-10,15,20-tri(N-methyl-3-pyridyl)-porphyrin chloride; or 5-(4-benzoylamidophenyl)-10,15,20-tri(N-methyl-3-quinolyl)-porphyrin chloride.

53. The compound of claim 51, where a metal is coordinated to said compound such that said compound has a formula: 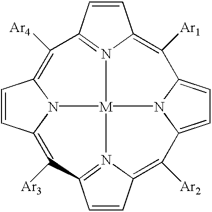
where M is 2H⁺ or a metal ion selected from the group consisting of Ca, Sc, Mn, Fe, Co, Ni, Cu, Zn, Sr, Y, Ru, Pd, Ag, In, Ba, La, Pt, Au, Mg, TiO, VO, Sn, Al, Ga, Er, Gd, Yb, Lu, Pr, Tb and Eu.

54. A method of modifying telomerase or telomere function, comprising contacting a thiaporphyrin or a selenaporphyrin with telomeric DNA wherein the thiaporphyrin or the selenaporphyrin has a formula: 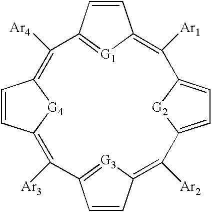
where one of G₁, G₂, G₃, or G₄ is S or Se, and the remainder are N,N, and NH; or
where two of G₁, G₂, G₃, or G₄ are either both S or both Se, two are N, and the two N are located opposite each other;
where Ar₁, Ar₂, Ar₃ and Ar₄ are H or independently 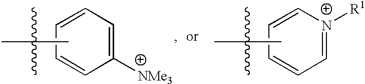
where R¹ is H, lower alkyl, CH₂CH₂OH, CH₂OAc, or CH₂CH₂CH₂SO₃⁻, 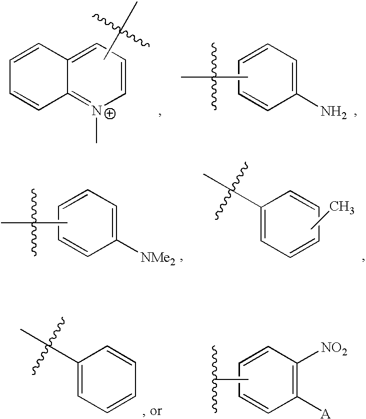
where A is H, OH, OMe, Cl or Me, 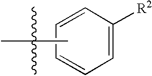
where R² is CO₂H, CONH₂, CONHCH₂CH₂Br or NHCOCH₃;
or where Ar₁, Ar₂, Ar₃ and Ar₄ are independently 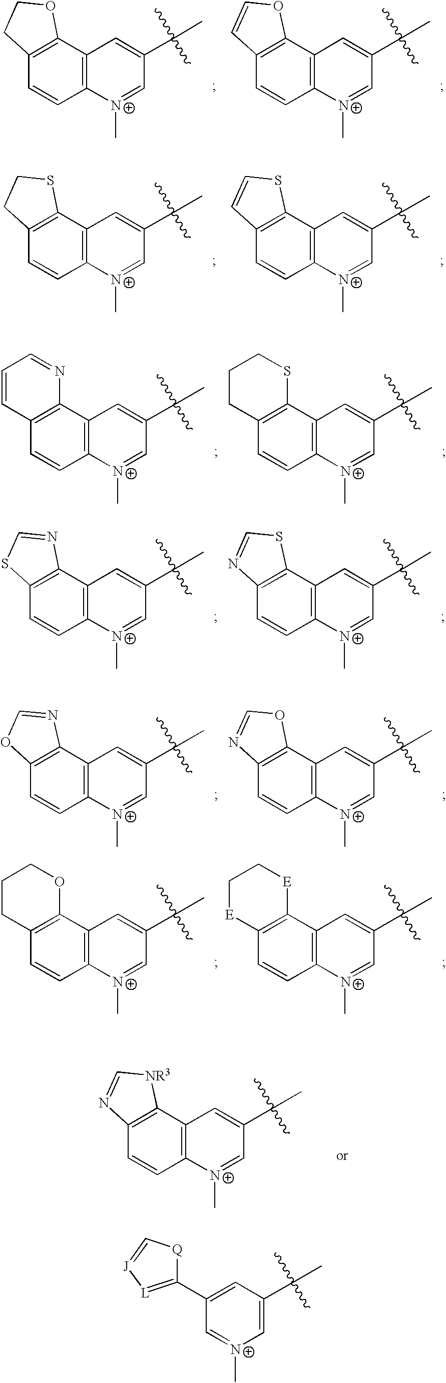
where Q is O, S, NH or NMe; J is CN or N; L is N or CH;
where R³ is lower alkyl, and each E is independently CH₂, NH, NMe, O or S;
or where Ar₁, Ar₂, Ar₃ and Ar₄ are independently 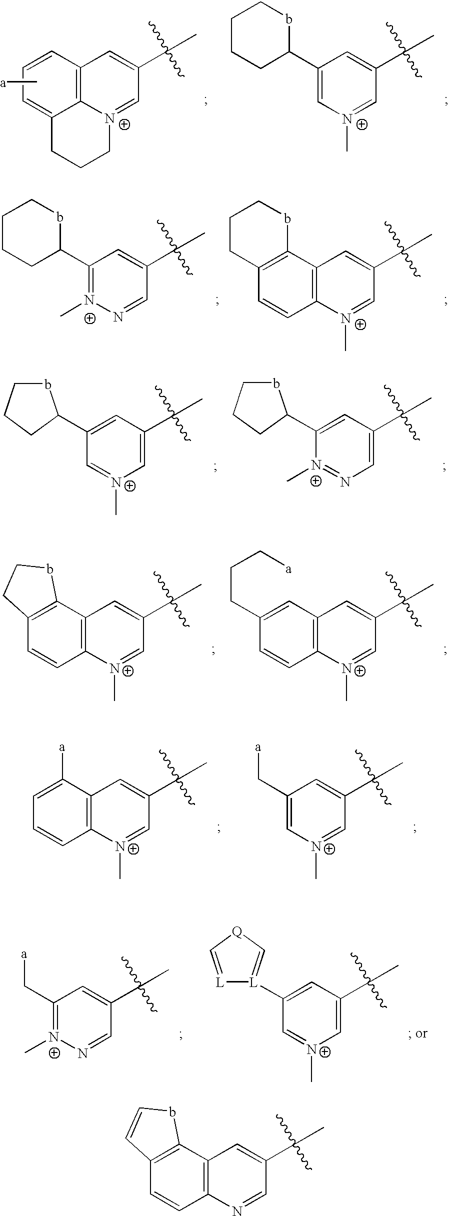
where a is NH₂, NHMe, NMe₂, OH, OMe, Sme; b is NH, NMe, SMe, O or S; Q is O, S, NH or NMe; each L is independently N or CH
or where Ar₁, Ar₂, Ar₃ and Ar₄ are independently 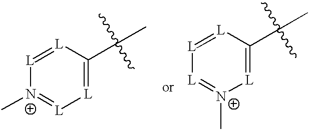
where each L is independently N or CH;
or where Ar₁, Ar₂, Ar₃ and Ar₄ are independently 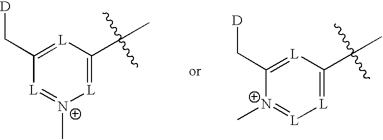
where each L is independently N or CH and D is NH₂, NHMe, NMe₂, OH, SH, SMe or CF₃;
or where Ar₁, Ar₂, Ar₃ and Ar₄ are independently 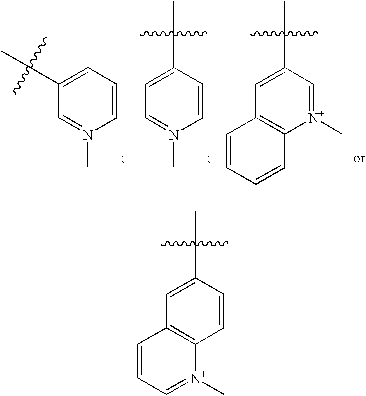
or where at least one, but not more than two, of Ar₁, Ar₂, Ar₃, or Ar₄ is 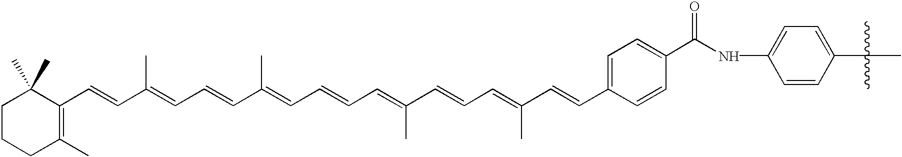
and the remainder of Ar₁, Ar₂, Ar₃, or Ar₄ are positively charged moieties;
or where at least one, but not more than two, of Ar₁, Ar₂, Ar₃, or Ar₄ is 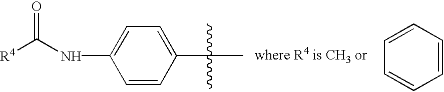
and the remainder of Ar₁, Ar₂, Ar₃, or Ar₄ are positively charged moieties.

55. A method of modifying telomerase or telomere function, comprising contacting a thiaporphyrin or a selenaporphyrin with telomeric DNA wherein the thiaporphyrin or the selenaporphyrin has a formula:: 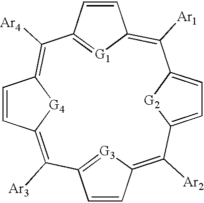
where one of G₁, G₂, G₃, or G₄ is S or Se, and the remainder are N,N, and NH; or
where two of G₁, G₂, G₃, or G₄ are either both S or both Se, two are N, and the two N are located opposite each other;
where Ar₁, Ar₂, Ar₃and Ar₄ are independently 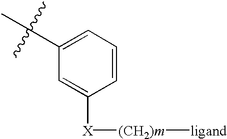
where m is 0-3, X is O, NH, CO, or CH₂, and where ligand is:

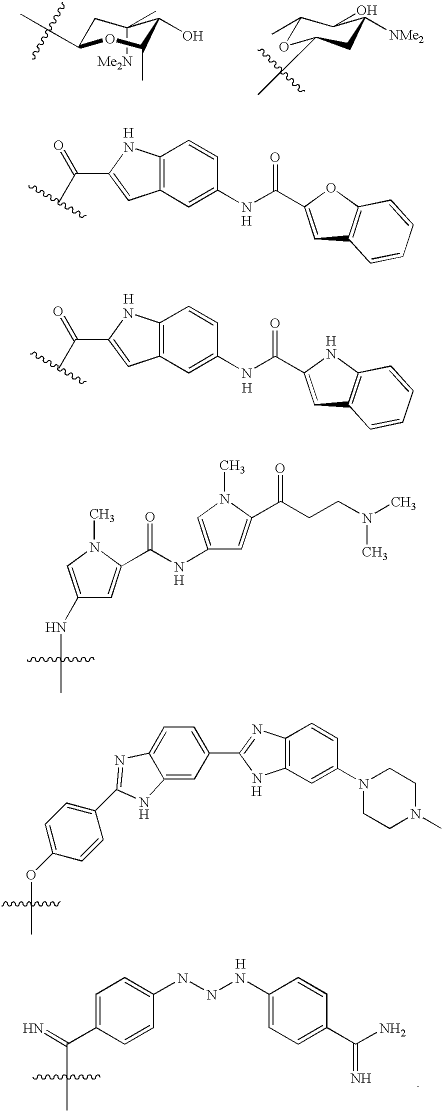

56. A method modifying telomerase or telomere function, comprising contacting a thiaporphyrin or a selenaporphyrin with telomeric DNA wherein the thiaporphyrin or selenaporphyrin has a formula: 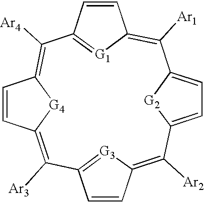
where one of G₁, G₂, G₃, or G₄ is S or Se, and the remainder are N,N, and NH; or
where two of G₁, G₂, G₃, or G₄ are either both S or both Se, two are N, and the two N are located opposite each other;
and where Ar₁, Ar₂, Ar₃, and Ar₄ are positively charged moieties, and assume a nonplanar disposition with regard to said thiaporphyrin structure or said selenaporphyrin structure.
